(19) United States
(12) Patent Application Publication
(10) Pub. No.: US 2011/0014191 A1
(43) Pub. Date: Jan. 20, 2011

(54) BREAST CANCER EXPRESSION PROFILING

(75) Inventors: Francois Bertucci, Carnoux-en-Provence (FR); Daniel Birnbaum, Marseille (FR); Pascal Finetti, Marseille (FR)

Correspondence Address: YOUNG & THOMPSON, 209 Madison Street, Suite 500, Alexandria, VA, 22314, US

(73) Assignees: IPSOGEN, Marseille (FR); INSTITUT PAOLI-CALMETTES, Marseille (FR)

(21) Appl. No.: 12/810,576

(22) PCT Filed: Dec. 24, 2008
(86) PCT No.: PCT/IB2008/003622

§ 371 Date: Sep. 29, 2010

Related U.S. Application Data
(60) Provisional application No. 61/009,395, filed on Dec. 28, 2007.

(51) Int. Cl. A61K 39/395; C12Q 1/68; C40B 40/06; C40B 30/04; C12Q 1/48; A61P 35/00

(52) U.S. Cl. 424/133.1; 435/6; 506/16; 506/9; 435/15

(57) ABSTRACT

The present invention relates to a method for analyzing cancer. e.g., breast cancer including detection of differential expression of at least one of the 16 genes encoding serine/threonine kinases listed in Table 1, or of the 16 genes, and to a polynucleotide library including at least one the 16 genes. This finds use in the development of novel applications, in particular in the development of prognosis or diagnostic of breast cancer or for monitoring the treatment of a patient with a breast cancer.

### TECHNICAL FIELD

The present invention relates to a method for analyzing cancer comprising detection of differential expression of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or of said 16 genes.

It finds many applications in particular in the development of prognosis or diagnostic of cancer or for monitoring the treatment of a patient with a cancer.

In the description which follows, the references between brackets [ ] refer to the attached reference list.

All the documents cited herein in the reference list are incorporated by reference in the texte below.

### STATE OF THE ART

Breast cancer (BC) is a heterogeneous disease whose clinical outcome is difficult to predict and treatment is not as adapted as it should be. BC can be defined at the clinical, histological, cellular and molecular levels. Efforts to integrate all these definitions improve our understanding of the disease and its management (Charafe-Jauffret E, Ginestier C, Monville F, et al. How to best classify breast cancer: conventional and novel classifications (review). Int J Oncol 2005; 27:1307-13 [1]). Initial studies using DNA microarrays have identified five major BC molecular subtypes (luminal A and B, basal, ERBB2-overexpressing and normal-like) (Perou C M, Sorlie T, Eisen M B, et al. Molecular portraits of human breast tumours. Nature 2000; 406:747-52; Sorlie T, Perou C M, Tibshirani R, et al. Gene expression patterns of breast carcinomas distinguish tumor subclasses with clinical implications. Proc Natl Acad Sci USA 2001; 98:10869-74; Sorlie T, Tibshirani R, Parker J, et al. Repeated observation of breast tumor subtypes in independent gene expression data sets. Proc Natl Acad Sci USA 2003; 100:8418-23; Bertucci F, Finetti P, Rougemont J, et al. Gene expression profiling identifies molecular subtypes of inflammatory breast cancer. Cancer Res 2005; 65:2170-8 [2-5]). These subtypes, which are defined by the specific expression of an intrinsic set of almost 500 genes, are variably associated with different histological types and with different prognosis. Luminal A BCs, which express hormone receptors, have an overall good prognosis and can be treated by hormone therapy. ERBB2-overexpressing BCs, which overexpress the ERBB2 tyrosine kinase receptor, have a poor prognosis and can be treated by targeted therapy using trastuzumab or lapatinib (Geyer C E, Forster J, Lindquist D, et al. Lapatinib plus capecitabine for HER2-positive advanced breast cancer. N Engl J Med 2006; 355:2733-43; Hudis C A. Trastuzumab—mechanism of action and use in clinical practice. N Engl J Med 2007; 357:39-51 [6,7]). No specific therapy is available against the other subtypes although the prognosis of basal and luminal B tumors is poor. This biologically relevant taxonomy remains imperfect since clinical outcome may be variable within each subtype, suggesting the existence of unrecognized subgroups.

Progress can be made in several directions. First, it is necessary to identify among good prognosis tumors such as luminal A BCs the ones that will relapse and metastasize. Second, a better definition of poor prognosis BCs and associated target genes will allow the development of new drugs that will in turn allow a better management of these cancers.

The human kinome constitutes about 1.7% of all human genes (Manning G, Whyte D B, Martinez R, Hunter T, Sudarsanam S. The protein kinase complement of the human genome. Science 2002; 298:1912-34 [8]), and represents a great part of genes whose alteration contributes to oncogenesis (Futreal P A, Coin L, Marshall M, et al. A census of human cancer genes. Nat Rev Cancer 2004; 4:177-83 [9]). Protein kinases mediate most signal transduction pathways in human cells and play a role in most key cell processes. Some kinases are activated or overexpressed in cancers, and constitute targets for successful therapies (Krause D S, Van Etten R A. Tyrosine kinases as targets for cancer therapy. N Engl J Med 2005; 353:172-87 [10]). In parallel to ongoing systematic sequencing projects (Stephens P, Edkins S, Davies H, et al. A screen of the complete protein kinase gene family identifies diverse patterns of somatic mutations in human breast cancer. Nat Genet 2005; 37:590-2 [11]), analysis of differential expression of kinases in cancers may identify new oncogenic activation pathways. As such, kinases represent an attractive focus for expression profiling in two important subtypes of BC.

So, evolution remains difficult to predict within some subtypes such as luminal A BC, and treatment is not as adapted as it should be. Refinement of prognostic classification and identification of new therapeutical targets are needed.

### DISCLOSURE OF THE INVENTION

The authors of the present invention have now discovered, entirely unexpectedly, that the expression of genes encoding certain serine/threonine kinases involved in mitosis, allows distinguishing subgroups of cancers, e.g. two subgroups of breast cancer, more particularly luminal A breast cancer: luminal Aa, of good prognosis, and luminal Ab, of poor prognosis.

Surprisingly, the authors also found that this set of genes is sufficient to distinguish basal from luminal A tumors, e.g., cancers.

So, in a first aspect, the invention relates to a method of analyzing cancer, advantageously breast cancer, comprising detecting differential expression of at least one of the 16 genes encoding serine/threonine kinases listed in Table 1.

In other words the present invention relates to a method for analyzing cancer, advantageously breast cancer, comprising detection of differential expression of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or of said 16 genes.

Table 1 indicates the name of each gene with its gene symbol, the kinase activity, and for each gene the relevant sequence(s) defining the gene (identification numbers: SEQ ID NO.). The present invention defines the nucleotide sequences by the different genes but it may cover also a definition of the polynucleotide sequences by the name of the gene or fragments thereof.

TABLE 1
List of the 16 kinases from the gene cluster overexpressed in luminal Ab subgroup as compared with luminal Aa
subgroup.

| | | | | | | | RefSeq | | |
|---|---|---|---|---|---|---|---|---|---|
| Probe | Kinase | p- | Gene | | | | Transcript | Chrom. | References |
| Set ID | Activity | Value** | Symbol | Names | Regulation | SEQ ID NO. | ID | Loc. | for drugs |
| 208079_s_at | Serine/ | 206E−10 | AURKA | Aurora kinase A, | Mitosis early | SEQ ID NO. | NM_003600 | 20q13.2-q13.3 | see Carvajal |
| | Thre- | | | STK6, STK15 | phases, | 17 | | | et al., 2006 |
| | onine | | | | centrosome | | | | |
| 209464_at | Serine/ | 245E−15 | AURKB | Aurora kinase B, | Mitosis late | SEQ ID NO. | NM_004217 | 17p13.1 | see Carvajal |
| | Thre- | | | STK12 | phases, | 20 | | | et al., 2006 |
| | onine | | | | cytokinesis | | | | |
| 209642_at | Serine/ | 384E−12 | BUB1 | Budding uninhibited | Spindle | SEQ ID NO. | NM_004336 | 2q14 | see de Carcer |
| | Thre- | | | by benzimidazoles 1 | assembly | 18 | | | et al. 2007 |
| | onine | | | homolog (yeast) | checkpoint | | | | |
| 203755_at | Serine/ | 607E−14 | BUB1B | Budding uninhibited | Spindle | SEQ ID NO. | NM_001211 | 15q15 | see de Carcer |
| | Thre- | | | by benzimidazoles 1 | assembly | 19 | | | et al. 2007 |
| | onine | | | homolog beta (yeast), | checkpoint | | | | |
| | | | | BUBR1 | | | | | |
| 203213_at | Serine/ | 464E−18 | CDC2 | Cell division cycle 2, | Cyclin | SEQ ID NO. | NM_001786 | 10q21.1 | see de Carcer |
| | Thre- | | | G1 to S and G2 to M, | complexes | 21 | | | et al. 2007 |
| | onine | | | CDK1 | in G2/M | | | | |
| 204510_at | Serine/ | 838E−08 | CDC7 | Cell division cycle 7 | S phase | SEQ ID NO. | NM_003503 | 1p22 | see de Carcer |
| | Thre- | | | (S. cerevisiae) | pre- | 23 | | | et al. 2007 |
| | onine | | | | replicative | | | | |
| | | | | | complexes | | | | |
| 205394_at | Serine/ | 513E−12 | CHEK1 | CHK1 checkpoint | S and G2 | SEQ ID NO. | NM_001274 | 11q24-q24 | see de Carcer |
| | Thre- | | | homolog (S. pombe) | phases, | 22 | | | et al. 2007 |
| | onine | | | | DNA | | | | |
| | | | | | damage | | | | |
| | | | | | checkpoint | | | | |
| 228468_at | Serine/ | 865E−08 | MASTL | Microtubule- | Mitosis | SEQ ID NO. | NM_032844 | 10p12.1 | |
| | Thre- | | | associated | | 24 | | | |
| | onine | | | serine/threonine | | | | | |
| | | | | kinase-like | | | | | |
| 204825_at | Serine/ | 230E−10 | MELK | Maternal embryonic | G2/M | SEQ ID NO. | NM_014791 | 9p13.2 | |
| | Thre- | | | leucine zipper kinase, | transition, | 27 | | | |
| | onine | | | pEg3 | pre-mRNA | | | | |
| | | | | | splicing | | | | |
| 204641_at | Serine/ | 685E−23 | NEK2 | NIMA (never in | Spindle | SEQ ID NO. | NM_002497 | 1q32.2-q41 | see de Carcer |
| | Thre- | | | mitosis gene a)- | assembly | 25 | | | et al. 2007 |
| | onine | | | related kinase 2 | checkpoint, | | | | |
| | | | | | centrosome | | | | |
| 219148_at | Serine/ | 157E−12 | PBK | PDZ binding kinase, | Mitosis | SEQ ID NO. | NM_018492 | 8p21.2 | |
| | Thre- | | | TOBK | | 28 | | | |
| | onine | | | | | | | | |
| 202240_at | Serine/ | 250E−15 | PLK1 | Polo-like kinase 1 | Spindle | SEQ ID NO. | NM_005030 | 16p12.1 | see Strebhardt |
| | Thre- | | | (Drosophila) | assembly | 26 | | | and Ullrich, |
| | onine | | | | checkpoint, | | | | 2006 |
| | | | | | centrosome | | | | |
| 204886_at | Serine/ | 167E−10 | PLK4 | Polo-like kinase 4 | Centrosome | SEQ ID NO. | NM_014264 | 4q27-q28 | see Strebhardt |
| | Thre- | | | (Drosophila), SAK | | 30 | | | and Ullrich, |
| | onine | | | | | | | | 2006 |
| 202200_s_at | Serine/ | 147E−07 | SRPK1 | SFRS protein kinase 1 | Pre-mRNA | SEQ ID NO. | NM_003137 | 6p21.3-p21.2 | |
| | Argi- | | | | splicing | 32 | | | |
| | nine | | | | | | | | |
| 204822_at | Serine/ | 588E−12 | TTK | TTK (tramtrack) | Spindle | SEQ ID NO. | NM_003318 | 6q13-q21 | see de Carcer |
| | Thre- | | | protein kinase, MPS1 | assembly | 29 | | | et al. 2007 |
| | onine | | | | checkpoint | | | | |
| | and | | | | | | | | |
| | Tyro- | | | | | | | | |
| | sine | | | | | | | | |
| 203856_at | Serine/ | 205E−09 | VRK1 | Vaccinia-related | S phase, P53 | SEQ ID NO. | NM_003384 | 14q32 | |
| | Thre- | | | kinase 1 | pathway | 31 | | | |
| | onine | | | | | | | | |

*Parameters for the QT clustering was from 15 genes for minimum cluster size, with a minimum correlation of r = 0.70.
**p-Value for t.test, to assume gene significance to separate both LuminalA groups.

In a particular embodiment, the invention relates to a method for analyzing breast cancer comprising detection of differential expression of the 16 genes encoding serine/threonine kinases listed in Table 1.

In other words, the method of the invention is a method for analyzing a breast cancer based on the analysis of the over or under expression of genes in a breast tissue sample, said analysis comprising the detection of at least one of the 16 genes mentioned above.

By “genes”, in the sense of the present invention, is meant a polynucleotide sequence, e.g., isolated, such as deoxyribonucleic acid (DNA), and, where appropriate, ribonucleic acid (RNA). The sequence of the genes may be the sequences SEQ ID NO. 17-32, or any complement sequence. This sequence may be the complete sequence of the gene, or a subsequence of the gene which would be also suitable to perform the method of the analysis according to the invention. A person skilled in the art may choose the position and length of the gene by applying routine experiments. The term should also be understood to include, as equivalents, analogs of RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. DNA may be obtained from said nucleic acids sample and RNA may be obtained by transcription of said DNA. In addition, mRNA may be isolated from said nucleic acids sample and cDNA may be obtained by reverse transcription of said mRNA.

By <<differential expression>>, in the sense of the present invention, is meant the difference between the level of expression of a gene in a normal tissue, i.e. a breast tissue free of cancer, and the level of expression of the same gene in the sample analysed.

Thus, the detection of differential expression of genes is the analysis of over or underexpression of polynucleotide sequences on a biological sample. Advantageously, this analysis comprises the detection of the overexpression and underexpression of at least one or more genes as described above.

By <<overexpression>>, in the sense of the present invention, is meant a level of expression that is higher than the level of a reference sample, for example a sample of breast tissue free of breast cancer.

By <<underexpression>>, in the sense of the present invention, is meant a level of expression that is lesser than the level of a reference sample, for example a sample of breast tissue free of breast cancer.

The over or under expression may be determined by any known method of the prior art. It may comprise the detection of difference in the expression level of the polynucleotide sequences according to the present invention in relation to at least one reference. Said reference comprises for example polynucleotide sequence(s) from sample of the same patient or from a pool of patients afflicted with luminal breast cancer, or from a pool of sample as described in Finetti et al. (Finetti P., Cervera N, Charafe-Jauffret E., Chabannon C., Charpin C, Chaffanet M., Jacquemier J., Viens P., Birnbaum D., Bertucci F. Sixteen kinase gene expression identifies luminal breast cancers with poor prognosis. Cancer Res. 2008; 68: (3); 1-10 [27]), or selected among reference sequence(s) which may be already known to be over or under expressed. The expression level of said reference can be an average or an absolute value of reference polynucleotide sequences. These values may be processed in order to accentuate the difference relative to the expression of the polynucleotide of the invention.

The analysis of the over or underexpression of polynucleotide sequences can be carried out on sample such as biological material derived from any mammalian cells, including cell lines, xenografts, human tissues preferably breast tissue, etc. The method according to the invention may be performed on sample from a patient or an animal.

Advantageously, the overepxression of at least one sequence is detected simultaneously to the underexpression of others sequences. “Simultaneously” means concurrent with or within a biologic or functionally relevant period of time during which the over expression of a sequence may be followed by the under expression of another sequence, or conversely, e.g., because both expressions are directly or indirectly correlated.

The number of sequences according to the various embodiments of the invention may vary in the range of from 1 to the total number of sequences described therein, e.g., 2, 3, 4, 5, 6, 7, 8, 9, 10, 11, 12, 13, 14, 15, 16 sequences.

In a particular embodiment of the invention, the differential gene expression separates basal and luminal A breast cancer.

By <<basal breast cancer>>, in the sense of the present invention, is meant a Basal-phenotype or basal-like breast cancers, characterized by specific molecular profile based on a gene list defined in Sorlie et al. [3], incorporated herein by reference. The specific molecular profile may be high expression of keratins 5 and 17, and fatty acid binding protein 7.

By <<luminal A breast cancer>>, in the sense of the present invention, is meant a breast cancer characterized by molecular profile on a specific gene list defined in Sorlie et al. [3], incorporated herein by reference. The specific molecular profile may be high expression of the ERα gene GATA binding protein 3, X-box binding protein 1, trefoil factor 3, hepatocyte nuclear factor 3, and estrogen-regulated LIV-1.

Advantageously, the differential gene expression distinguishes subgroups of luminal A tumors of good or poor prognosis.

By <<subgroups>>, in the sense of the present invention, is meant groups of patients afflicted with luminal A breast cancer of good prognosis and groups of patients afflicted with luminal A breast cancer of poor prognosis.

By <<good prognosis>>, in the sense of the present invention, is meant luminal A tumors (Aa cases) characterized by low mitotic activity as compared to other luminal A tumors (Ab cases). Good prognosis may also refer to the scoring defined below and according to Finetti el al. ([27]), i.e. a negative kinase-score. A good prognosis may also indicate that the patient afflicted with luminal A breast cancer is expected to have no distant metastases within 5 years of initial diagnosis of cancer (i.e. relapse-free survival (RFS) superior to 5 years).

By <<low mitotic activity>>, in the sense of the present invention, is meant kinase-score value below 0 ([27]), i.e. a negative kinase-score. By <<poor prognosis>>, in the sense of the present invention, is meant luminal A tumors (Ab cases) characterized by high mitotic activity as compared to other luminal A tumors (Aa cases). Poor prognosis may also refer to the scoring defined below and according to Finetti el al. ([27]), i.e. a positive kinase-score. A poor prognosis may also indicate that the patient afflicted with luminal A breast cancer is expected to have some distant metastases within 5 years of initial diagnosis of cancer (i.e. relapse-free survival (RFS) superior to 5 years).

By <<high mitotic activity>>, in the sense of the present invention, is meant kinase-score value above 0 ([27]), i.e. a positive kinase-score.

In this embodiment of the invention, the subgroup of luminal A tumors of poor prognosis presents a higher mitotic activity compared with other luminal A tumors.

Advantageously, the method may comprise the determination of the expression level or overexpression level of AURKA and/or AURKB and/or PLK genes. The overexpression of these genes may be associated with a poor clinical outcome.

The method may comprise the determination of the expression level of AURKA gene, or AURKB gene, or PLK gene.

The method of the invention may comprise the determination of AURKA and PLK genes, or the determination of the expression level of AURKB and PLK genes, or the determination of the expression level of AURKA and AURKB genes, or the determination of the expression level of AURKA and AURKB and PLK genes.

In a particular embodiment of the invention, the detection is performed on nucleic acids from a tissue sample.

By <<tissue sample>>, in the sense of the present invention, is meant a sample of tissue, preferably breast tissue or a cell. If the tissue sample is breast tissue, it may come from invasive adenocarcinoma.

In another embodiment of the invention, the detection is performed on nucleic acids from a tumor cell line.

By <<tumor cell line>>, in the sense of the present invention, is meant cell line derived from a cancer cell obtained from a patient.

In a particular embodiment of the invention, the dermination of the expression level of the gene(s) disclosed herein may be performed by various methods well-known in the art, e.g., real-time PCR (polymerase chain reaction), including 5′nuclease TaqMan® (Roche), Scorpions® (DxS Genotyping) (Whitcombe, D., Theaker J., Guy, S. P., Brown, T., Little, S. (1999)—Detection of PCR products using self-probing amplicons and flourescence. Nature Biotech 17, 804-807 [35]) or Molecular Beacons™ (Abravaya K, Huff J, Marshall R, Merchant B, Mullen C, Schneider G, and Robinson J (2003) Molecular beacons as diagnostic tools: technology and applications. Clin Chem Lab Med 41, 468-474 [36]).

In another embodiment of the invention, the detection is performed on DNA microarrays.

By <<DNA microarrays>>, in the sense of the present invention, is meant an arrayed series of thousands of microscopic spots of DNA oligonucleotides, each containing picomoles of a specific DNA sequence chosen among the genes of the invention. This DNA oligonucleotide is used as probes to hybridize a cDNA or cRNA sample (called target) under high-stringency conditions. Probe-target hybridization is usually detected and quantified by fluorescence-based detection of fluorophore-labeled targets to determine relative abundance of nucleic acid sequences in the target.

In standard microarrays, the probes are attached to a solid surface by a covalent bond to a chemical matrix (via epoxy-silane, amino-silane, lysine, polyacrylamide or others).

The cDNA oligonucleotide probes (also called “probeset”) that may be used to hybridyze a DNA or RNA sample corresponding to one or more of the 16 genes encoding serine/threonine kinases as defined above are defined in Table 2.

TABLE 2

| Gene | | Probeset | SET |
|---|---|---|---|
| symbol | Name | sequence | number |
| AURKA | Aurora kinase A, STK6, | SEQ ID NO. 1, | 1 |
| | STK15 | SEQ ID NO. 33-43 | |
| AURKB | Aurora kinase B, STK12 | SEQ ID NO. 2, | 2 |
| | | SEQ ID NO. 44-54 | |
| BUB1 | Budding uninhibited by | SEQ ID NO. 3, | 3 |
| | benzimidazoles 1 homolog | SEQ ID NO. 55-65 | |
| | (yeast) | | |
| BUB1B | Budding uninhibited by | SEQ ID NO. 4, | 4 |
| | benzimidazoles 1 homolog | SEQ ID NO. 66-76 | |
| | beta (yeast), BUBR1 | | |
| CDC2 | Cell division cycle 2, G1 | SEQ ID NO. 5, | 5 |
| | to S and G2 to M, CDK1 | SEQ ID NO. 77-87 | |
| CDC7 | Cell division cycle 7 | SEQ ID NO. 6, | 6 |
| | (S. cerevisiae) | SEQ ID NO. 88-98 | |
| CHEK1 | CHK1 checkpoint homolog | SEQ ID NO. 7, | 7 |
| | (S. pombe) | SEQ ID NO. 99-109 | |
| MASTL | Microtubule-associated | SEQ ID NO. 8, | 8 |
| | serine/threonine kinase-like | SEQ ID NO. 110-120 | |
| MELK | Maternal embryonic leucine | SEQ ID NO. 9, | 9 |
| | zipper kinase, pEg3 | SEQ ID NO. 121-131 | |
| NEK2 | NIMA (never in mitosis | SEQ ID NO. 10, | 10 |
| | gene a)-11related kinase 2 | SEQ ID NO. 132-142 | |
| PBK | PDZ binding kinase, TOBK | SEQ ID NO. 11, | 11 |
| | | SEQ ID NO. 143-153 | |
| PLK1 | Polo-like kinase 1 | SEQ ID NO. 12, | 12 |
| | (Drosophila) | SEQ ID NO. 154-164 | |
| PLK4 | Polo-like kinase 4 | SEQ ID NO. 13, | 13 |
| | (Drosophila), SAK | SEQ ID NO. 165-175 | |
| SRPK1 | SFRS protein kinase 1 | SEQ ID NO. 14, | 14 |
| | | SEQ ID NO. 176-186 | |
| TTK | TTK (tramtrack) protein | SEQ ID NO. 15, | 15 |
| | kinase, MPS1 | SEQ ID NO. 187-197 | |
| VRK1 | Vaccinia-related kinase 1 | SEQ ID NO. 16, | 16 |
| | | SEQ ID NO. 198-208 | |

The cDNA oligonucleotide probesets that may be used to hybridyze a DNA or RNA sample corresponding to one or more of the 16 genes encoding serine/threonine kinases, can be any sequence between 3′ and 5′ end of the polynucleotide sequence(s) of the corresponding SET as defined in Table 2, allowing a complete detection of the implicated genes.

In order to detect the expression of a determined gene described above, at least one probeset sequence or subsequence of the corresponding SET may be used.

By “cDNA subsequence of the gene”, in the sense of the invention, is meant a sequence of nucleic acids of cDNA total sequence of the gene that allows a specific hybridization under stringent conditions, as an example more than 10 nucleotides, preferably more than 15 nucleotides, and most preferably more than 25 nucleotides, as an example more than 50 nucleotides or more than 100 nucleotides.

In other words, the method of the invention may comprise the detection of at least one, or at least two or three polynucleotide sequence(s) or subsequence(s), or a complement thereof, selected in the SETS defined in Table 2.

Another aspect of the invention is to provide a polynucleotide library that molecularly characterizes cancer comprising or corresponding to at least one of the 16 genes encoding serine/threonine kinases listed in Table 1.

The polynucleotide library of the invention may comprise, or may consist of, at least one polynucleotide sequence allowing the detection of a corresponding at least one gene of the 16 genes encoding serine/threonine kinases listed in Table 1.

In other words, an aspect of the invention relates to a polynucleotide library that molecularly characterizes a cancer, comprising or corresponding to polynucleotide sequence(s) allowing the detection of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or to said 16 genes.

The polynucleotide library of the invention may comprise, or may consist of at least one, or at least 2 or 3, polynucleotide sequence(s) or subsequence(s), or complement(s) thereof, selected in at least one SET of Table 2, allowing the detection of a corresponding at least one gene of the 16 genes encoding serine/threonine kinases listed in Table 1. In a particular aspect of the invention, the invention relates to polynucleotide library that molecularly characterizes a cancer comprising or corresponding to the 16 genes encoding serine/threonine kinases listed in Table 1. In this embodiment, the polynucleotide library of the invention may comprise, or may consist of, polynucleotide sequences allowing the detection of the 16 genes encoding serine/threonine kinases listed in Table 1.

For example, in this case, the polynucleotide library of the invention may comprise, or may consist of at least one, or at least 2 or 3, polynucleotide sequence(s) or subsequence(s), or complement(s) thereof, selected in each SET of Table 2.

By <<corresponding to>>, in the sense of the present invention, is meant a polynucleotide library that consists of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or of said 16 genes.

In a particular embodiment of the invention, the library is immobilized on a solid support.

Such a solid support may be selected from the group comprising at least one of nylon membrane, nitrocellulose membrane, glass slide, glass beads, membranes on glass support or silicon chip, plastic support.

Another aspect of the invention is to provide a method of prognosis or diagnostic of breast cancer or for monitoring the treatment of a patient with a breast cancer comprising the implementation of the method of analyzing breast cancer as described above on nucleic acids from a patient.

Such a method is the use of a method for analyzing breast cancer as described above for prognosis or diagnostic of breast cancer or for monitoring the treatment of a patient with a breast cancer comprising the implementation of the method of analyzing breast cancer as described above on nucleic acids from a patient.

Another aspect of the invention is to provide a method for analysing differential gene expression associated with breast cancer disease, comprising:

a) obtaining a polynucleotide sample from a patient,

b) reacting said polynucleotide sample obtained in step (a) with a polynucleotide library as defined above, and

c) detecting the reaction product of step (b).

In other words, the invention provides a method for analysing differential gene expression associated with breast cancer disease, comprising:

a) reacting a polynucleotide sample from the patient with the polynucleotide library as defined above, and

b) detecting a reaction product of step (b).

A differential gene expression “associated with” breast cancer refers to an underexpression or a overexpression of a nucleic acid caused by, or contributed to by, or causative of a breast cancer.

By “reacting a polynucleotide sample with the polynucleotide library”, in the sense of the invention, is meant contacting the nucleic acids of the sample with polynucleotide sequences in conditions allowing the hybridization of cDNA or mRNA total sequence of the gene or of cDNA or mRNA subsequences or of primers of the gene with polynucleotide sequences of the library.

By “reaction product” in the sense of the present invention, is meant the product resulting of the hybridization between the polynucleotide sample from the patient with the polynucleotide library as defined above.

The detection of the reaction product of step (b) may be quantitative, related to the transcript expression level.

In a particular embodiment of the invention, the method for analysing differential gene expression associated with breast cancer disease further comprises:

a) obtaining a reference polynucleotide sample,

b) reacting said reference sample with said polynucleotide library, for example by hybridising the polynucleotide sample with the polynucleotide library as defined above,

c) detecting a control sample reaction product, and

d) comparing the amount of said polynucleotide sample reaction product to the amount of said control sample reaction product.

By <<reference polynucleotide sample>>, in the sense of the present invention, is meant one or more biological samples from a cell, a tissue sample or a biopsy from breast. Said reference may be obtained from the same female mammal than the one to be tested or from another female mammal, preferably from the same specie, or from a population of females mammal, preferably from the same specie, that may be the same or different from the test female mammal or subject. Said control may correspond to a biological sample from a cell, a cell line, a tissue sample or a biopsy from breast.

The step d) of comparison of the amount of said polynucleotide sample reaction product to the amount of said reference sample reaction product may be performed by any method well-known in the art.

For example, the method may comprise the following steps:

a) comparing molecular profile from breast cancer samples (e.g. 50, 100 or more, e.g., 138 breast cancers samples) based on polynucleotide library associated to kinome according to the gene list defined as covering all the kinase family according, e.g., to Manning et al. [8],

b) identifying a specific polynucleotides cluster (e.g. with 5, 10 or 16 kinase genes) by unsupervised Quality Threshold cluster analyses as described in Finetti et al. [27], where gene expression were observed differential among the luminal A breast cancers,

c) computing a score using mean of the kinase genes combined with normalisation parameters, to assess the classification of luminal A breast cancers.

By “kinome” is meant the ensemble of kinases proteins that are expressed in a particular cell or tissue or present in the genome of an organism.

Another aspect of the invention is a method for classifying a patient, e.g., a female patient, afflicted with a breast cancer as having a luminal A breast cancer with relapse-free survival (RFS) superior to 5 years (luminal Aa breast cancer) or as having a luminal A breast cancer with RFS inferior to 5 years (luminal Ab breast cancer), comprising the steps of:

a) calculating the kinase score (KS) based on the expression of at least one gene, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 kinases, or on said 16 kinases listed in Table 1 or their expression product, of the sample of said patient, distinguishing the subgroups luminal Aa and luminal Ab, and,

b) classifying said patient as having luminal Aa breast cancer when the kinase score is negative, or classifying patient as having luminal Ab when the kinase score is positive.

By “Kinase Score (KS)”, in the sens of the invention, is meant a score which is based on the expression level of 16 kinase genes. It was defined as:

$$KS = \frac{A}{n} {\sum }_{i = 1}^{n} ( xi - B )$$

where A and B represent normalization parameters, which make the KS comparable across the different datasets, n the number of available kinase genes (7 to 16), and xi the logarithmic gene expression level in tumor i. Using a cut-off value of 0, each tumor was assigned a low score (KS<0, i.e. with overall low expression of 16 kinase genes) or a high score (KS>0, i.e. with overall strong expression of 16 kinase genes). In the present invention, the number of available kinase genes, i.e. n, is from 1 to 16.

The method of the invention allows the prediction of the clinical outcome of patient afflicted with luminal A, by classifying these patients in luminal Aa or luminal Ab patients.

Another aspect of the invention is to provide a method for screening molecule for treating luminal A cases of poor prognosis comprising the analysis of the action of said molecule on at least one the 16 kinases listed in table 1 or their expression.

In other words, the invention relates to a method for screening molecule for treating luminal A cases of poor prognosis comprising the analysis of the action of said molecule on at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 kinases listed in table 1 or their expression, or on said 16 kinases.

In a particular aspect of the invention, the invention relates to a method for screening molecule for treating luminal A cases of poor prognosis comprising the analysis of the action of said molecule on at least one, or at least two, or at least three, or more, e.g., all of the 16 kinases listed in table 1 or their expression product.

By <<the action of said molecule>>, in the sense of the present invention, is meant the positive effect of the molecule on the survival of the patient, or on the RFS of the patient, the reduction of size of the tumor, or the diminution of the expression of the kinase.

Another aspect of the invention is to provide a kit comprising the polynucleotide library as described above, for carrying out a method of the invention, i.e. a method for analyzing breast cancer, a method for analysing differential gene expression associated with breast cancer, or a method for screening molecule for treating luminal A cases of poor prognosis.

A kit of the invention may contain sets of polynucleotide sequences of the library as well as control samples. The kit may also contain test reagents necessary to perform the pre-hybridization, hybridization, washing steps and hybridization detection.

Another aspect of the invention is a method for treating a patient with a breast cancer. This method comprises i) implementing a method of analysing of differential gene expression profile according to the present invention on a sample from said patient, and ii) determining a treatment for this patient based on the analysis of differential gene expression profile obtained with said method. “Treating” encompasses treating as well as ameliorating at least one symptom of the condition or disease.

Another aspect of the invention is a method for predicting clinical outcome for a patient diagnosed with cancer, comprising determining the expression level of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes listed in Table 1, or all of the 16 genes of Table1, or their expression products, in a cancer tissue obtained from the patient, normalized against a reference gene or genes, and compared to the amount found in a reference cancer tissue set, wherein overexpression of the group of genes predicts a poor clinical outcome.

By “clinical outcome” in the sens of the invention, is meant the survival, the partial remission, the total remission, the time to progression of the disease or the relapse of the disease. By “clinical outcome”, it may be also meant the evolution of luminal A breast cancer to luminal Aa or luminal Ab breast cancer.

The poor clinical outcome may be measured in terms of relapse-free survival (RFS). A poor clinical outcome may indicate that the patient afflicted by luminal A breast cancer is expected to have some distant metastases within 5 years of initial diagnosis of cancer.

This method may be used to predict clinical outcome of patient diagnosed with a breast cancer, or a colon cancer, or a lung cancer, or a prostate cancer, or a hepatocellular cancer, or a gastric cancer, or a pancreatic cancer, or a cervical cancer, or a ovarian cancer, or a liver cancer, or a bladder cancer, or a cancer of the urinary tract, or a thyroid cancer, or a renal cancer, or a carcinoma, or a melanoma, or a brain cancer.

Preferably, all of the methods of the invention may be applicable to the cancers listed above.

In a particular embodiment, the method may be used to predict clinical outcome of a patient diagnosed with breast cancer.

Advantageously, the method may comprise the determination of the expression level or overexpression level of AURKA and/or AURKB and/or PLK genes. The overexpression of these genes may be associated with a poor clinical outcome.

The method may comprise the determination of the expression level of AURKA gene, or AURKB gene, or PLK gene.

The method of the invention may comprise the determination of AURKA and PLK genes, or the determination of the expression level of AURKB and PLK genes, or the determination of the expression level of AURKA and AURKB genes, or the determination of the expression level of AURKA and AURKB and PLK genes.

Advantageously, the expression level of the genes may be determined using RNA obtained from a frozen or fresh tissue sample.

The expression level may be determined by reverse phase polymerase chain reaction (RT-PCR).

Another object of the invention is a method of predicting the likelihood of the recurrence of cancer following treatment in a cancer patient, comprising determining the expression level of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes listed in Table 1, or all of the 16 genes of Table1, or their expression products, in a cancer tissue obtained from the patient, normalized against a control gene or genes, and compared to the amount found in a reference cancer tissue set, wherein overexpression of the group of genes indicates increased risk of recurrence following treatment.

The cancer analyzed by the method of the invention may be breast cancer, or colon cancer, or lung cancer, or prostate cancer, or hepatocellular cancer, or gastric cancer, or pancreatic cancer, or cervical cancer, or ovarian cancer, or liver cancer, or bladder cancer, or cancer of the urinary tract, or thyroid cancer, or renal cancer, or carcinoma, melanoma, or brain cancer.

Advantageously, the cancer may be breast cancer.

The expression level may be determined before any surgical removal of tumor, or may be determined following surgical removal of tumor, i.e. removal of cancer.

The expression level may be determined using RNA obtained from a fresh or frozen sample.

The expression level may be determined by reverse phase polymerase chain reaction (RT-PCR).

The method of predicting the likelihood of the recurrence of cancer may follow the treatment of the cancer with one or more kinase inhibitor drugs, e.g., serine and/or threonine kinase inhibitor drugs, e.g., the following drugs: MK0457, PHA-739358, MLN8054, AZD1152, ON01910, BI2536, flavopiridol, USN-01, ZM447-439 (AstraZeneca, MK0457 (Merck), AZD1152 (AstraZeneca), PHA-680632, MLN8054 (Millenium Pharmaceutical), PHA739358 (Nerviano Sciences), scytonemin, BI2536, ON01910 as described in Carvajal D., Tse Archie, Schwartz G. Aueora kinases: new targets for cancer therapy. Clin. Cancer Res 2006; 12(23) ([33]) and Strebhardt K., Ullrich A. Targeting polo-like kinase 1 for cancer therapy. Nature 2006, Vol. 6, 321-330 ([34]), the content of which is incorporated herein by reference.

Another object of the invention is a kit comprising one or more of (1) extraction buffer/reagents and protocol; (2) reverse transcription buffer/reagents and protocol; and (3) quantitative PCR buffer/reagents and protocol suitable for performing a method of the invention.

Advantageously, the kit may comprise a data retrieval and analysis software.

Advantageously, the kit may comprise pre-designed primers.

Advantageously, the kit may comprise pre-designed PCR probes and primers.

Another object of the invention is a method for predicting, for example in vitro, the therapeutic success of a given mode of treatment in a subject having cancer, comprising

(i) determining the pattern of expression levels of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or of said 16 genes,

(ii) comparing the pattern of expression levels determined in (i) with one or several reference pattern(s) of expression levels,

(iii) predicting therapeutic success for said given mode of treatment in said subject from the outcome of the comparison in step (ii).

Advantageously, the cancer may be selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

Advantageously, the cancer may be breast cancer.

The given mode of treatment (i) may act on cell proliferation, and/or (ii) may act on cell survival, and/or (iii) may act on cell motility; and/or (iv) may comprise administration of a chemotherapeutic agent.

The given mode of treatment may be E7070, PHA-533533, hymenialdisine, NU2058 & NU6027, AZ703, BMS-387032, CYC202 (R-roscovitine), CDKi277, NU6140, PNU-252808, RO-3306, CVT-313, SU9516, Olomoucine, ZK-CDK (ZK304709), JNJ-7706621, PD0332991, PD0183812, Fascplysin, CA224, CINK4, caffeine, pentoxifylline, wortmannin, LY294002, UCN-01, debromohymenialdisine, Go6976, SB-218078, ICP-1, CEP-3891, TAT-S216A, CEP-6367, XL844, PD0166285, BI2536, ON01910, Scytonemin, wortmannin, HMN-214, cyclapolin-1, hesperadin, JNJ-7706621, PHA-680632, VX-680 (MK-0457), ZM447-439, MLN8054, R763, AZD1152, CYC116, SNS-314, MKC-1693, AT9283, quinazoline derivatives, MP235, MP529, cincreasin, SP600125 (de Carcer et al. Targeting cell cycle kinases for cancer therapy, Current Medicinal Chemistry, 2007, Vol. 14, No. 1; 1-17 [29], Malumbres et al. Current Opinion in genetics & Development 2007, 17:60-65 [30], Malumbres et al. Therapeutic opportunities to control tumor cell cycles, Clin. Transl. Oncol. 2006; 8(6):1-000 [31], Iressa (gefitnib, ZD1839, anti-EGFR, PDGFR, c-kit, Astra-Zeneca); ABX-EGFR (anti-EGFR, Abgenix/Amgen); Zamestra (FTI, J & J/Ortho-Biotech); Herceptin (anti-HER2/neu, Genentech); Avastin (bevancizumab, anti-VEGF antibody, Genentech); Tarceva (ertolinib, OSI-774, RTK inhibitor, Genentech-Roche); ZD66474 (anti-VEGFR, Astra-Zeneca); Erbitux (IMC-225, cetuximab, anti-EGFR, Imclone/BMS); Oncolar (anti-GRH, Novartis); PD-183805 (RTK inhibitor, Pfizer); EMD72000, (anti-EGFRNEGF ab, MerckKgaA); CI-1033 (HER2/neu & EGF-R dual inhibitor, Pfizer); EGF10004; Herzyme (anti-HER2 ab, Medizyme Pharmaceuticals); Corixa (Microsphere delivery of HER2/neu vaccine, Medarex), and the drugs listed in Awada et al., The Pipeline of new anticancer agents for breast cancer treatment in 2003, Critical Reviews in Oncology/Hematology 48 (2003), 45-63 ([32]), ZM447-439 (AstraZeneca, MK0457 (Merck), AZD1152 (AstraZeneca), PHA-680632, MLN8054 (Millenium Pharmaceutical), PHA739358 (Nerviano Sciences), scytonemin, BI2536, ON01910 ([33] and [34]).

The method of the invention may use a predictive algorithm.

Another object of the invention is a method of treatment of a neoplastic disease in a subject, comprising the steps of:

a) predicting therapeutic success for a given mode of treatment in a subject having cancer, e.g., breast cancer by any method of the invention,

b) treating said neoplastic disease in said patient by said mode of treatment, if said mode of treatment is predicted to be successful.

Another object of the invention is a method of selecting a therapy modality for a subject afflicted with a neoplastic disease, comprising

(i) obtaining a biological sample from said subject,

(ii) predicting from said sample, by any method of the invention, therapeutic success in a subject having cancer, e.g., breast cancer, for a plurality of individual modes of treatment,

(iii) selecting a mode of treatment which is predicted to be successful in step (ii).

Advantageously, the expression level may be determined:

(i) with a hybridization based method, or

(ii) with a hybridization based method utilizing arrayed probes, or

(iii) with a hybridization based method utilizing individually labeled probes, or

(iv) by real time PCR, or

(v) by assessing the expression of polypeptides, proteins or derivatives thereof, or (vi) by assessing the amount of polypeptides, proteins or derivatives thereof.

Other advantages may also appear to one skilled in the art from the non-limitative examples given below, and illustrated by the enclosed figures.

### BRIEF DESCRIPTION OF THE FIGURES

FIG. 1 represents the kinase gene expression profiling in luminal A and basal breast cancers. N Hierarchical clustering of 138 BC samples (80 luminal A and 58 basal; left panel), 8 cell lines (3 luminal epithelial mammary cell lines, 3 basal epithelial mammary cell lines and 2 lymphocytic cell lines; right panel) and 435 unique kinase probe sets. Each row represents a gene and each column represents a sample. The expression level of each gene in a single sample is relative to its median abundance across the 138 BC samples and is depicted according to a color scale shown at the bottom. In the right panel, genes are in the same order as in the left panel. Yellow and blue indicate expression levels respectively above and below the median. The magnitude of deviation from the median is represented by the color saturation. In the right panel, genes are in the same order as in the left panel. The dendrograms of samples (above matrix) represent overall similarities in gene expression profiles and are zoomed in B. Colored bars to the right indicate the location of 4 gene clusters of interest that are zoomed in C. B/Dendrogram of samples. Top, Dendrogram of BC samples (left) and cell lines (right): two large groups of BC samples are evidenced by clustering and delimited by dashed orange vertical line. Bottom, molecular subtype of samples (red, basal; blue, luminal A; green, lymphocytic cell lines). See the near perfect separation of basal and luminal A BCs (p=1.13 10-36; Fisher's exact test). C/Expanded view of the four selected genes clusters. The first cluster is the 16 kinase gene cluster identified by QT-clustering. See its expression homogeneous in basal samples, but rather heterogeneous in luminal A samples.

FIG. 2 represents the identification and validation of two prognostic subgroups of luminal A BC samples based on the 16 kinase-gene set. A/Classification of our 80 luminal A BCs using the 16 kinase genes. Genes are in the same order than in the cluster in FIG. 1C. Tumor samples are ordered from left to right according to the decreasing Kinase Score (KS). The dashed orange line indicates the threshold 0 that separates the two classes of samples, luminal Ab with positive KS (at the left of the line, black horizontal class) and luminal Aa with negative KS (right to the line, blue horizontal class). Legend is as in FIG. 1. B/Kaplan-Meier relapse-free survival in our series of luminal Aa (L.Aa), luminal Ab (L.Ab) and basal (B.) breast cancers. Basal medullary breast cancers were excluded from survival analyses. The p-values are calculated using the log-rank test. C/Classification of luminal A BCs from three public data sets using the 16 kinase genes: Wang et al [15], Loi et al [16], van de Vijver et al [14]. The legend is similar to FIG. 2A. D/Kaplan-Meier relapse-free survival in the three pooled series of luminal Aa (L.Aa), luminal Ab (L.Ab) and basal (B.) breast cancers. The legend is similar to FIG. 2B.

FIG. 3 represents the kinase Score in breast cancers. A/Box plots of the Kinase Score (KS) in each molecular subtype (left) and each luminal A subgroup (right) across a total of 1222 tumors. Median and range are indicated. NA means samples without any assigned subtype. Under the box plots, are the 5-year RFS for each subtype and for each KS-based subgroup in each subtype. Medullary breast cancers—all basal and one normal-like—were excluded from survival analyses. The p-values are calculated using the log-rank test. B/Classification of 1222 tumors based on the Kinase Score (KS). The molecular subtype of samples is indicated as follows: dark blue for luminal Aa, black for luminal Ab, light blue for luminal B, pink for ERBB2-overexpressing, red for basal, green for normal-like, and white for unassigned. Samples are ordered from left to right according to their increasing KS.

FIG. 4 shows the gene expression profiling of a series of breast cancer and their classification in molecular subtypes. A/Hierarchical clustering of 227 BC samples (91 luminal A, and 67 basal, as well as other subtypes; left panel), and 435 unique kinase probe sets. Each row represents a gene and each column represents a sample. The expression level of each gene in a single sample is relative to its median abundance across the 227 BC samples and is depicted according to a color scale shown at the bottom. In the right panel, genes are in the same order as in the left panel. Red and green indicate expression levels respectively above and below the median. The magnitude of deviation from the median is represented by the color saturation. In the right panel, genes are in the same order as in the left panel. The dendrograms of samples (above matrix) represent overall similarities in gene expression profiles and are zoomed in B. Colored bars to the right indicate the location of 11 gene clusters of interest that are zoomed in C. B/Dendrograms of samples. Top, Dendrograms of BC samples (left) and cell lines (right): two large groups of BC samples are evidenced by clustering and delimited by dashed orange vertical line. Bottom, molecular subtype of samples (red, basal; blue, luminal A; green, lymphocytic cell lines).

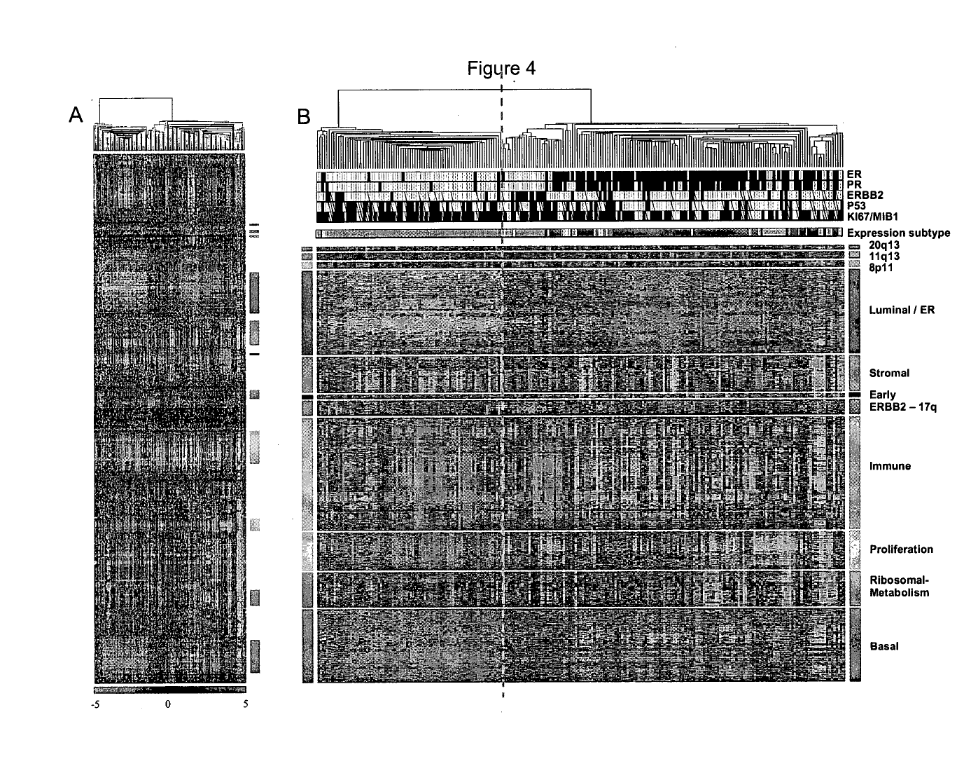

FIG. 5 is a schematic representation of basal and luminal subtypes in a continuum of balanced proliferation and differentiation. The most proliferative breast cancers are the basal ones whereas the most differentiated are the luminal Aa tumors. Above are listed transcription factors that are crucial for luminal differentiation and biology. Horizontal lines proposes appropriate treatments.

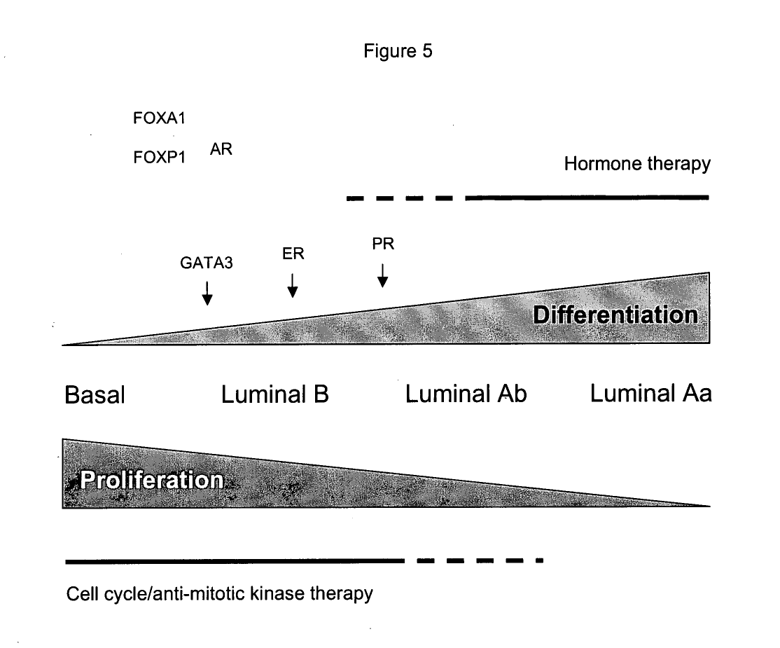

### DETAILED DESCRIPTION OF THE INVENTION

Breast cancer (BC) is a heterogeneous disease made of various molecular subtypes with different prognosis. However, evolution remains difficult to predict within some subtypes such as luminal A, and treatment is not as adapted as it should be. Refinement of prognostic classification and identification of new therapeutical targets are needed. Using oligonucleotide microarrays, we profiled 227 BCs. We focused our analysis on two major BC subtypes with opposite prognosis, luminal A (n=80) and basal (n=58), and on genes encoding protein kinases. Whole-kinome expression separated luminal A and basal tumors. The expression (measured by a Kinase Score KS) of 16 genes encoding serine/threonine kinases involved in mitosis distinguished two subgroups of luminal A tumors: Aa, of good prognosis, and Ab, of poor prognosis. This classification and its prognostic impact were validated in 276 luminal A cases from three independent series profiled across different microarray platforms. The classification outperformed the current prognostic factors in univariate and multivariate analyses in both training and validation sets. The luminal Ab subgroup, characterized by high mitotic activity as compared to luminal Aa tumors, displayed clinical characteristics and a KS intermediate between the luminal Aa subgroup and the luminal B subtype, suggesting a continuum in luminal tumors. Some of the mitotic kinases of the signature represent therapeutical targets under investigation. The identification of luminal A cases of poor prognosis should help select appropriate treatment, while the identification of a relevant kinase set provides potential targets.

Our study focused on the kinome of luminal A and bc cancers, whose relevance to cancer biology and therapeutics is well established (Manning G, Whyte D B, Martinez R, Hunter T, Sudarsanam S. The protein kinase complement of the human genome. Science 2002; 298:1912-34 [8]). To our knowledge, this is the first study of profiling and exclusive and comprehensive analysis of kinase genes in bc.

### The Breast Cancer Kinome Differs Between Luminal A and Basal Subtypes

As an exploratory step, we applied hierarchical clustering to 435 kinase genes. We found that luminal A and basal tumors had different global kinome expression patterns, with some degree of transcriptional heterogeneity within luminal A tumors. This observation suggests differential expression of many kinases, and consequently different phosphorylation programs between the two subtypes. Global clustering revealed broad coherent kinase clusters corresponding to cell processes (proliferation, differentiation) or to cell type (immune response), with overxepression of the proliferation cluster in basal samples and of the differentiation cluster in luminal A samples.

### Mitotic Kinases Identify Two Subgroups of Luminal A Breast Cancers

Interestingly, a Kinase Score (KS) based on their expression distinguished two subgroups of luminal A tumors (Aa and Ab) with different survival. Identified in our tumor series, this classification and its prognostic impact were validated in 276 luminal A cases from three independent series profiled across different microarray platforms. Importantly, the KS outperformed the current prognostic factors in uni- and multivariate analyses in both training and validation sets.

Analysis of molecular function and biological processes revealed that the prognostic value of this kinase signature is mainly related to proliferation. Indeed, the 16 genes encode kinases involved in G2 and M phases of the cell cycle. Aurora-A and -B are two major kinases regulating mitosis and cytokinesis, respectively. BUB1 (budding inhibited by benzimidazole), BUB1B, CHEK1 (checkpoint kinase 1), PLK1 (polo-like kinase 1), NEK2 (never in mitosis kinase 2) and TTK/MPS1 play key roles in the various cell division checkpoints. PLK4 (polo-like kinase 4) is involved in centriole duplication. CDC2/CDK1 is a major component of the cell cycle machinery in association with mitotic cyclins. CDC7, MELK (maternal embryonic leucine zipper kinase) and VRK1 (vaccinia-related kinase 1) are regulators of the S/G2 and G2/M transitions. SRPK1 regulates splicing. Not much is known about MASTL and PBK kinases.

Prognostic gene expression signatures related to grade (Sotiriou C, Wirapati P, Loi S, et al. Gene expression profiling in breast cancer: understanding the molecular basis of histologic grade to improve prognosis. J Natl Cancer Inst 2006; 98:262-72; Ivshina A V, George J, Senko O, et al. Genetic reclassification of histologic grade delineates new clinical subtypes of breast cancer. Cancer Res 2006; 66:10292-301 [18, 19]) or proliferation (Dai H, van't Veer L, Lamb J, et al. A cell proliferation signature is a marker of extremely poor outcome in a subpopulation of breast cancer patients. Cancer Res 2005; 65:4059-66 [20]) have been reported. We found respectively 8 and 10 of our 16 kinase genes in the lists of genes differentially expressed in grade I vs grade III BCs reported by Sotiriou et al (97 genes) and Ivshina et al (264 genes). Three kinase genes, AURKA, AURKB, and BUB1, are included in a prognostic set of 50 cell cycle-related genes [20], and AURKB is one of the 5 proliferation genes included in the Recurrence Score defined by Paik et al (Paik S, Shak S, Tang G, et al. A multigene assay to predict recurrence of tamoxifen-treated, node-negative breast cancer. N Engl J Med 2004; 351:2817-26 [21]). Furthermore, proliferation appears to be the most prominent predictor of outcome in many other published prognostic gene expression signatures (Desmedt C, Sotiriou C. Proliferation: the most prominent predictor of clinical outcome in breast cancer. Cell Cycle 2006; 5:2198-202 [22]). This link of our signature with proliferation also explains the correlation of our luminal A subgrouping with histological grade, which is in part based on a mitotic index. But interestingly, comparison with Ki67 and grade showed that our mitotic kinase signature performed better in identifying these tumors and predicting the survival of patients.

### Mitotic Kinases as Therapeutic Targets

Targeting cell proliferation is a main objective of anticancer therapeutic strategies. Kinases have proven to be successful targets for therapies. Mitotic kinases have stimulated intense work focused on identifying novel antimitotic drugs. Some of them included in our signature represent targets under investigation (Miglarese M R, Carlson R O. Development of new cancer therapeutic agents targeting mitosis. Expert Opin Investig Drugs 2006; 15:1411-25 [23]). For example, targeting of Aurora kinases is a promising way of treating tumors (Carvajal R D, Tse A, Schwartz G K. Aurora kinases: new targets for cancer therapy. Clin Cancer Res 2006; 12:6869-75 [24]). Clinical trials of four Aurora kinase inhibitors are ongoing in the United States and Europe: MK0457 and PHA-739358 inhibit Aurora-A and Aurora-B, MLN8054 selectively inhibits Aurora-A, and AZD1152 selectively inhibits Aurora-B. Similarly, small-molecule inhibitors of PLK1 such as ON01910 and BI2536 are being tested (Strebhardt K, Ullrich A. Targeting polo-like kinase 1 for cancer therapy. Nat Rev Cancer 2006; 6:321-30 [25]), as well as flavopiridol (inhibitor of the cyclin-dependant kinase CDC2), and UCN-01 (inhibitor of CHEK1). Other less studied but potential therapeutic targets include TTK, BUB and NEK proteins (de Carcer G, de Castro I P, Malumbres M. Targeting cell cycle kinases for cancer therapy. Curr Med Chem 2007; 14:969-85 [26]).

### A New Relevant Subgroup of Luminal A Breast Cancers

Despite their relatively good prognosis as compared to luminal B tumors, luminal A tumors display a heterogeneous clinical outcome after treatment, which generally includes hormone therapy. It is important to define the cases that may evolve unfavorably, all the more so that different types of hormone therapy, chemotherapy, and targeted molecular therapy are available. Our poor prognosis subgroup of luminal A tumors (Ab cases) is characterized by high mitotic activity as compared to other luminal A tumors (Aa cases). Any error in the key steps in division regulated by these kinases—centrosome duplication, spindle checkpoint, microtubule-kinetochore attachment, chromosome condensation and segregation, cytokinesis—may lead to aneuploïdy and progressive chromosomal instability. This may in part explain the high grade and poor prognosis of these tumors.

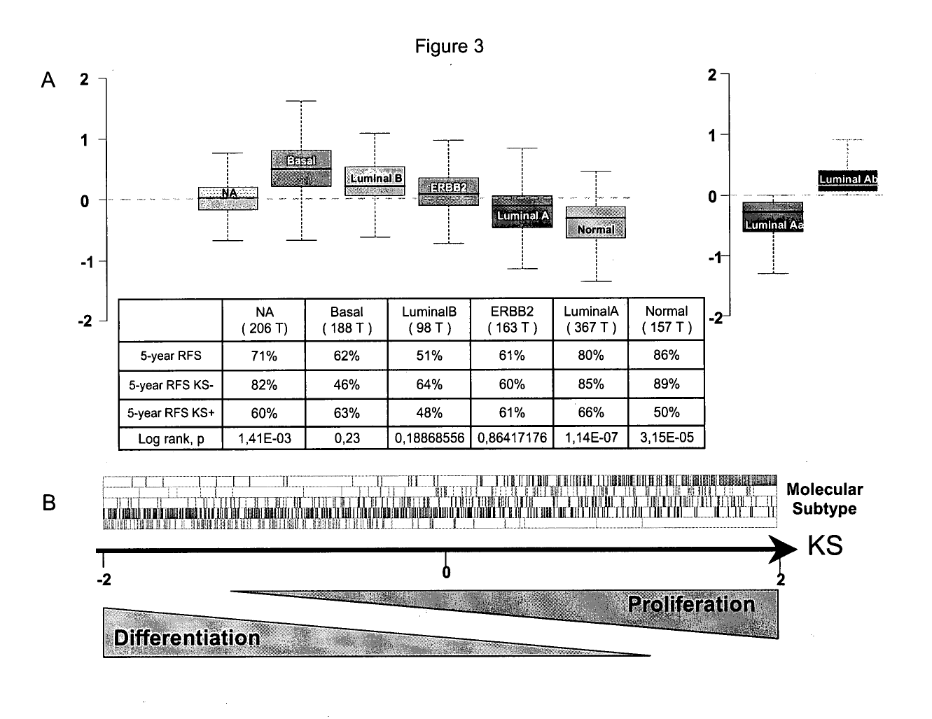

In fact, the luminal Ab subgroup displayed clinical characteristics and a KS intermediate between the luminal Aa subgroup and the luminal B subtype. These subgroups were not previously recognized by the Sorlie's intrinsic gene set. We interpret this finding as follows. The use of intrinsic set distinguishes a large proportion of luminal B cancers but is unable to pick all proliferative cases. A small proportion of cases is left to cluster with the luminal A cases, and are therefore labeled luminal A. An explanation for the poor efficacy of Sorlie's set to define all proliferative luminal cases may be the low number of genes involved in proliferation, including a very low number of kinases. Our mitotic kinase signature makes possible to identify all proliferative luminal cases, and reveals a continuum of luminal cases from the more proliferative (luminal B) to the less proliferative (luminal Aa). Reciprocally, there may be a gradient of luminal differentiation giving a continuum of luminal BCs, including, from poorly-differentiated to highly-differentiated, luminal B, Ab and Aa (FIG. 3B). Optimal response to hormone therapy would be obtained with luminal Aa BCs, whereas luminal B and Ab would benefit from chemotherapy and/or new drugs targeting the cell cycle and various kinases as discussed above.

### EXAMPLES

### Materials and Methods

### Patients and Samples

A total of 227 pre-treatment early breast cancer samples were available for RNA profiling on Affymetrix microarrays. They were collected from 226 patients with invasive adenocarcinoma who underwent initial surgery at the Institut Paoli-Calmettes and Hôpital Nord (Marseille) between 1992 and 2004. Samples were macrodissected by pathologists, and frozen within 30 min of removal in liquid nitrogen. All profiled specimens contained more than 60% of tumor cells. Characteristics of samples and treatment are listed in Supplementary Table 1.

SUPPLEMENTARY TABLE 1
Clinico-biological information on 227 tumors

| | | | No. Patients | |
|---|---|---|---|---|
| | | | (percent of evaluated cases) | |
| | Age | Median | Total (N = 227) | |
| Characteristics* | (year) | (range) | 52 (24-85) | |
| Pathological type | (226) | CAN | 183 | (81%) |
| | | MED | 22 | (10%) |
| | | MIX | 9 | (4%) |
| | | LOB | 12 | (5%) |
| Grade SBR | (226) | I | 22 | (10%) |
| | | II | 55 | (24%) |
| | | III | 149 | (66%) |
| Pathological axillary | (213) | Positive | 123 | (58%) |
| lymph node status | | Negative | 90 | (42%) |
| Pathological tumor | (176) | pT1 | 53 | (30%) |
| Size | | pT2 | 84 | (48%) |
| | | pT3 | 39 | (22%) |
| IHC ER status | (227) | Positive | 108 | (48%) |
| | | Negative | 119 | (52%) |
| IHC PR status | (227) | Positive | 90 | (40%) |
| | | Negative | 137 | (60%) |
| IHC P53 status | (177) | Positive | 66 | (37%) |
| | | Negative | 111 | (63%) |
| IHC ERBB2 | (205) | Positive | 36 | (18%) |
| | | Negative | 169 | (82%) |
| IHC Ki67/MIB1 status | (187) | Positive | 142 | (76%) |
| | | Negative | 45 | (24%) |

*In parentheses are numbers of evaluated cases among 227 tumors.
CAN: Ductal, MED: Medullary, MilX: Mixed, LOB: Lobular, tumor size
pT1: <=2 cm, pT2: <=5 cm and pT3: >5 cm

In addition, we profiled RNA extracted from 8 cell lines that provided models for cell types encountered in mammary tissues: 3 luminal epithelial cell lines (HCC1500, MDA-MB-134, ZR-75-30), 3 basal epithelial cell lines (HME-1, HMEC-derived 184B5, MDA-MB-231), and 2 lymphocytic B and T cell lines (Daudi and Jurkatt, respectively). All cell lines were obtained from ATCC (Rockville, Md.—http://www.atcc.org/) and were grown as recommended

Gene Expression Profiling with DNA Microarrays

Gene expression analyses were done with Affymetrix U133 Plus 2.0 human oligonucleotide microarrays containing over 47,000 transcripts and variants, including 38,500 well-characterized human genes. Preparation of cRNA from 3 μg total RNA, hybridizations, washes and detection were done as recommended by the supplier (Affymetrix). Scanning was done with Affymetrix GeneArray scanner and quantification with Affymetrix GCOS software. Hybridization images were inspected for artifacts.

### Gene Expression Data Analysis

Expression data were analyzed by the RMA (Robust Multichip Average) method in R software (Brian D. Ripley. The R project in statistical computing. MSOR Connections. The newsletter of the LTSN Maths, Stats & OR Network., 1(1):23-25, February 2001 [28] and http://www.r-project.org/doc/bib/R-other_bib.html#R:Ripley:2001 using Bioconductor and associated packages (Irizarry R A, Hobbs B, Collin F, et al. Exploration, normalization, and summaries of high density oligonucleotide array probe level data. Biostatistics 2003; 4:249-64 [12]). Before analysis, a filtering process removed from the dataset the genes with low and poorly measured expression as defined by expression value inferior to 100 units in all 227 breast cancer tissue samples, retaining 31189 genes/ESTs.

Before unsupervised hierarchical clustering, a second filter excluded genes showing low expression variation across the 227 samples, as defined by standard deviation (SD) inferior to 0.5 log 2 units (only for calculation of SD, values were floored to 100 since discrimination of expression variation in this low range can not be done with confidence), retaining 14486 genes/ESTs. Data was then log 2-transformed and submitted to the Cluster program (Eisen M B, Spellman P T, Brown P O, Botstein D. Cluster analysis and display of genome-wide expression patterns. Proc Natl Acad Sci USA 1998; 95:14863-8 [13]) using data median-centered on genes, Pearson correlation as similarity metric and centroid linkage clustering. Results were displayed using TreeView program [13]. Quality Threshold (QT) clustering identifies sets of genes with highly correlated expression patterns among the hierarchical clustering. It was applied to the kinase probe sets and basal and luminal A tumors using TreeView program [13]. The cut-offs for minimal cluster size and minimal correlation were 15 and 0.7, respectively. The gene clusters were interrogated using Ingenuity software (Redwood City, Calif., USA) to assess significant representation of biological pathways and functions.

### Definition of Kinase-Encoding Probe Sets

The kinome database established by Manning et al [8] was used as reference to extract the kinase-encoding genes from the Affymetrix Genechip U133 Plus 2.0. First, because annotation of the HUGO (Human Genome Organisation) symbols did not correspond necessarily between the genes represented on the Affymetrix chip and the kinome, we used the mRNA accession number as cross-reference. cDNA sequences of the kinome were compared with the representative mRNA sequences of the Unigene database using BLASTn, and alignements between these sequences were obtained. All mRNAs with exact match were retained, and their accession number compared with those of the 31,189 selected probe sets given by Affymetrix. Second, some kinase genes were represented by several probe sets on the Affymetyrix chip. This may introduce bias in the weight of the groups of genes for analysis by QT-clustering. In these cases, probe sets with an extension <<_at>>, next <<s_at>> and followed by all other extensions were preferentially kept. When several probe sets with the best extension were available, the one with the highest median value was retained. From the initial list of 518 kinases, we finally retained 435 probe sets representing 435 kinase genes.

### Collection of Published Datasets

To test the performance of our multigene signature in other BC samples, we analyzed three major publicly available data sets: van de Vijver et al (van de Vijver M J, He Y D, van't Veer U, et al. A gene-expression signature as a predictor of survival in breast cancer. N Engl J Med 2002; 347:1999-2009 [14]), Wang et al Wang Y, Klijn J G, Zhang Y, et al. Gene-expression profiles to predict distant metastasis of lymph-node-negative primary breast cancer. Lancet 2005; 365:671-9 (Wang Y, Klijn J G, Zhang Y, et al. Gene-expression profiles to predict distant metastasis of lymph-node-negative primary breast cancer. Lancet 2005; 365:671-9 [15]) collected from NCBI/Genbank GEO database (series entry GSE2034), and Loi et al (Loi S, Haibe-Kains B, Desmedt C, et al. Definition of clinically distinct molecular subtypes in estrogen receptor-positive breast carcinomas through genomic grade. J Clin Oncol 2007; 25:1239-46 [16]) collected from NCBI/Genbank GEO database (series entry GSE6532). Analysis of each data set was done in several successive steps: identification of molecular subtypes based on the common intrinsic gene set, identification of the kinase gene set common with ours, followed by computing of the Kinase Score (see below) for the luminal A samples. Clinical data of luminal A samples from our series and public series used for analyses are detailed in Supplementary Table 3.

SUPPLEMENTARY TABLE 3
Histoclinical characteristics of 276 luminal A tumors from published datasets.

| | | | | | | | | | | ESR1 mRNA | |
|---|---|---|---|---|---|---|---|---|---|---|---|
| Data | Sample | Kinase | | SBR | Pathological | | Pathological axillary lymph | | Follow-up | expression | PGR mRNA |
| Set | Name | Group | Age (Years) | Grade | tumor Size | | node status | Relapse | (months) | level | expression level |
| Loi et | 1127 | Aa | 63 | II | >2 | cm | positive | no | 87.33 | rich | rich |
| al. | | | | | | | | | | | |
| Loi et | 1133 | Aa | 70 | I | <=2 | cm | positive | no | 66.92 | rich | poor |
| al. | | | | | | | | | | | |
| Loi et | 1142 | Ab | 61 | II | <=2 | cm | negative | no | 93.47 | rich | rich |
| al. | | | | | | | | | | | |
| Loi et | 1167 | Aa | 58 | NA | >2 | cm | negative | no | 95.93 | poor | rich |
| al. | | | | | | | | | | | |
| Loi et | 1193 | Aa | 68 | II | >2 | cm | negative | no | 84.4 | rich | rich |
| al. | | | | | | | | | | | |
| Loi et | 1301 | Ab | 52 | II | <=2 | cm | positive | no | 48.36 | rich | poor |
| al. | | | | | | | | | | | |
| Loi et | 1432 | Aa | 71 | I | <=2 | cm | positive | no | 84.01 | rich | rich |
| al. | | | | | | | | | | | |
| Loi et | 1889 | Aa | 76 | II | <=2 | cm | negative | no | 64.23 | rich | rich |
| al. | | | | | | | | | | | |
| Loi et | 1981 | Ab | 70 | II | >2 | cm | positive | no | 70.24 | rich | rich |
| al. | | | | | | | | | | | |
| Loi et | 2152 | Aa | 75 | NA | >2 | cm | negative | no | 2.17 | rich | rich |
| al. | | | | | | | | | | | |
| Loi et | 2175 | Aa | 77 | II | <=2 | cm | positive | no | 54.34 | rich | rich |
| al. | | | | | | | | | | | |
| Loi et | 2190 | Ab | 82 | NA | <=2 | cm | negative | no | 0.49 | rich | rich |
| al. | | | | | | | | | | | |
| Loi et | 4904 | Ab | 69 | I | >2 | cm | positive | yes | 68.01 | rich | poor |
| al. | | | | | | | | | | | |
| Loi et | 5428 | Ab | 69 | NA | >2 | cm | positive | no | 0.26 | rich | rich |
| al. | | | | | | | | | | | |
| Loi et | 555 | Aa | 66 | NA | >2 | cm | negative | no | 117.55 | rich | rich |
| al. | | | | | | | | | | | |
| Loi et | 595 | Ab | 56 | NA | <=2 | cm | negative | no | 114.86 | rich | rich |
| al. | | | | | | | | | | | |
| Loi et | 669 | Ab | 60 | III | >2 | cm | negative | no | 112.89 | rich | rich |
| al. | | | | | | | | | | | |
| Loi et | 680 | Ab | 61 | I | >2 | cm | positive | yes | 77.96 | rich | poor |
| al. | | | | | | | | | | | |
| Loi et | 711 | Ab | 67 | NA | >2 | cm | positive | yes | 32.03 | poor | poor |
| al. | | | | | | | | | | | |
| Loi et | 736 | Aa | 48 | I | <=2 | cm | positive | yes | 97.18 | rich | rich |
| al. | | | | | | | | | | | |
| Loi et | 738 | Aa | 74 | I | <=2 | cm | positive | no | 106.51 | rich | rich |
| al. | | | | | | | | | | | |
| Loi et | 742 | Ab | 67 | III | <=2 | cm | positive | no | 105.63 | poor | rich |
| al. | | | | | | | | | | | |
| Loi et | 112B55 | Ab | 61 | II | >2 | cm | positive | yes | 11.01 | rich | rich |
| al. | | | | | | | | | | | |
| Loi et | 114B68 | Ab | 67 | I | <=2 | cm | positive | no | 125.9 | rich | rich |
| al. | | | | | | | | | | | |
| Loi et | 130B92 | Aa | 73 | II | <=2 | cm | positive | yes | 52.96 | rich | rich |
| al. | | | | | | | | | | | |
| Loi et | 138B34 | Aa | 65 | I | >2 | cm | negative | no | 113.94 | rich | poor |
| al. | | | | | | | | | | | |
| Loi et | 139B03 | Ab | 84 | NA | >2 | cm | negative | yes | 3.94 | rich | poor |
| al. | | | | | | | | | | | |
| Loi et | 159B47 | Ab | 57 | II | <=2 | cm | negative | yes | 77.96 | rich | rich |
| al. | | | | | | | | | | | |
| Loi et | 162B98 | Aa | 73 | III | >2 | cm | negative | yes | 106.94 | rich | rich |
| al. | | | | | | | | | | | |
| Loi et | 166B79 | Ab | 65 | II | >2 | cm | negative | no | 117.88 | poor | rich |
| al. | | | | | | | | | | | |
| Loi et | 170B15 | Aa | 70 | II | >2 | cm | negative | yes | 48.92 | rich | rich |
| al. | | | | | | | | | | | |
| Loi et | 235C20 | Ab | 71 | I | >2 | cm | negative | no | 115.98 | rich | rich |
| al. | | | | | | | | | | | |
| Loi et | 244C89 | Ab | 51 | II | >2 | cm | positive | yes | 86.93 | poor | rich |
| al. | | | | | | | | | | | |
| Loi et | 254C80 | Aa | 67 | II | >2 | cm | negative | no | 112.95 | rich | rich |
| al. | | | | | | | | | | | |
| Loi et | 307C50 | Aa | 66 | II | >2 | cm | positive | no | 93.9 | rich | poor |
| al. | | | | | | | | | | | |
| Loi et | 48A46 | Aa | 78 | I | >2 | cm | negative | no | 21.95 | poor | poor |
| al. | | | | | | | | | | | |
| Loi et | 6B85 | Ab | 71 | I | >2 | cm | positive | yes | 7 | rich | poor |
| al. | | | | | | | | | | | |
| Loi et | 71A50 | Aa | NA | NA | NA | | negative | NA | 0 | rich | rich |
| al. | | | | | | | | | | | |
| Loi et | 84A44 | Ab | 84 | II | >2 | cm | positive | no | 74.94 | poor | poor |
| al. | | | | | | | | | | | |
| Loi et | 8B87 | Aa | 58 | I | <=2 | cm | negative | no | 118.97 | rich | poor |
| al. | | | | | | | | | | | |
| Loi et | 96A21 | Aa | 63 | II | >2 | cm | negative | yes | 2.99 | rich | rich |
| al. | | | | | | | | | | | |
| Loi et | 50108 | Aa | 69 | NA | <=2 | cm | positive | no | 174.55 | rich | rich |
| al. | | | | | | | | | | | |
| Loi et | 50110 | Aa | 56 | NA | >2 | cm | positive | no | 170.48 | rich | rich |
| al. | | | | | | | | | | | |
| Loi et | 50137 | Ab | 62 | NA | <=2 | cm | negative | yes | 110.23 | rich | poor |
| al. | | | | | | | | | | | |
| Loi et | 50153 | Aa | 59 | NA | <=2 | cm | positive | no | 173.27 | rich | rich |
| al. | | | | | | | | | | | |
| Loi et | 50172 | Aa | 61 | I | <=2 | cm | negative | no | 170.48 | rich | rich |
| al. | | | | | | | | | | | |
| Loi et | 50176 | Aa | 59 | II | >2 | cm | negative | yes | 30.46 | poor | poor |
| al. | | | | | | | | | | | |
| Loi et | 50178 | Ab | 63 | III | >2 | cm | NA | yes | 124.68 | rich | rich |
| al. | | | | | | | | | | | |
| Loi et | 50181 | Aa | 53 | I | <=2 | cm | negative | no | 158.23 | rich | rich |
| al. | | | | | | | | | | | |
| Loi et | 50182 | Ab | 70 | II | >2 | cm | negative | no | 163.88 | rich | poor |
| al. | | | | | | | | | | | |
| Loi et | 50183 | Aa | 77 | I | <=2 | cm | negative | no | 148.4 | rich | rich |
| al. | | | | | | | | | | | |
| Loi et | 50184 | Ab | 68 | II | <=2 | cm | negative | no | 118.01 | rich | rich |
| al. | | | | | | | | | | | |
| Loi et | 50188 | Aa | 71 | I | <=2 | cm | positive | no | 145.71 | rich | rich |
| al. | | | | | | | | | | | |
| Loi et | 50204 | Aa | 78 | II | <=2 | cm | NA | no | 146.56 | rich | poor |
| al. | | | | | | | | | | | |
| Loi et | 50211 | Ab | 63 | II | <=2 | cm | positive | yes | 98.69 | rich | rich |
| al. | | | | | | | | | | | |
| Loi et | 50219 | Ab | 65 | III | <=2 | cm | positive | no | 142.06 | rich | poor |
| al. | | | | | | | | | | | |
| Loi et | 50221 | Ab | 73 | III | >2 | cm | negative | no | 110.03 | rich | rich |
| al. | | | | | | | | | | | |
| Loi et | 50233 | Aa | 57 | I | <=2 | cm | negative | no | 151 | rich | rich |
| al. | | | | | | | | | | | |
| Loi et | 50236 | Aa | 72 | II | >2 | cm | positive | no | 74.35 | rich | rich |
| al. | | | | | | | | | | | |
| Loi et | 50237 | Aa | 79 | I | >2 | cm | positive | no | 146.33 | rich | rich |
| al. | | | | | | | | | | | |
| Loi et | 50239 | Aa | 62 | NA | <=2 | cm | negative | no | 51.71 | poor | poor |
| al. | | | | | | | | | | | |
| Loi et | 50251 | Aa | 70 | II | <=2 | cm | positive | yes | 123.24 | rich | rich |
| al. | | | | | | | | | | | |
| Loi et | 104 | Ab | 60 | NA | >2 | cm | negative | yes | 21.29 | rich | rich |
| al. | | | | | | | | | | | |
| Loi et | 1183 | Ab | 50 | I | <=2 | cm | negative | no | 52.27 | rich | rich |
| al. | | | | | | | | | | | |
| Loi et | 1248 | Aa | 70 | I | <=2 | cm | negative | no | 107.17 | rich | rich |
| al. | | | | | | | | | | | |
| Loi et | 145 | Aa | 45 | II | >2 | cm | positive | no | 154.87 | rich | rich |
| al. | | | | | | | | | | | |
| Loi et | 223 | Ab | 64 | III | >2 | cm | negative | yes | 61.6 | rich | rich |
| al. | | | | | | | | | | | |
| Loi et | 23 | Aa | 46 | II | <=2 | cm | positive | no | 156.78 | rich | rich |
| al. | | | | | | | | | | | |
| Loi et | 348 | Ab | 65 | II | >2 | cm | positive | yes | 7.26 | rich | rich |
| al. | | | | | | | | | | | |
| Loi et | 382 | Aa | 60 | III | >2 | cm | negative | no | 153.36 | rich | rich |
| al. | | | | | | | | | | | |
| Loi et | 484 | Aa | 64 | II | <=2 | cm | negative | no | 128.53 | rich | rich |
| al. | | | | | | | | | | | |
| Loi et | 485 | Aa | 64 | NA | <=2 | cm | NA | no | 149.52 | rich | rich |
| al. | | | | | | | | | | | |
| Loi et | 522 | Ab | 63 | NA | >2 | cm | negative | no | 117.72 | rich | poor |
| al. | | | | | | | | | | | |
| Loi et | 53 | Aa | 61 | NA | <=2 | cm | negative | no | 170.12 | rich | rich |
| al. | | | | | | | | | | | |
| Loi et | 535 | Aa | 59 | III | <=2 | cm | negative | no | 146.96 | rich | poor |
| al. | | | | | | | | | | | |
| Loi et | 544 | Aa | 54 | II | <=2 | cm | negative | no | 142.23 | rich | rich |
| al. | | | | | | | | | | | |
| Loi et | 549 | Aa | 64 | NA | <=2 | cm | positive | yes | 120.44 | rich | poor |
| al. | | | | | | | | | | | |
| Loi et | 573 | Aa | 63 | III | <=2 | cm | negative | no | 138.58 | rich | poor |
| al. | | | | | | | | | | | |
| Loi et | 90 | Aa | 61 | NA | <=2 | cm | negative | yes | 69.82 | rich | poor |
| al. | | | | | | | | | | | |
| Loi et | 93 | Aa | 58 | NA | <=2 | cm | negative | no | 165.22 | rich | rich |
| al. | | | | | | | | | | | |
| Loi et | 125B43 | Ab | NA | NA | NA | | negative | NA | 0 | rich | rich |
| al. | | | | | | | | | | | |
| Loi et | 140B91 | Aa | 61 | II | <=2 | cm | negative | no | 92.88 | rich | rich |
| al. | | | | | | | | | | | |
| Loi et | 151B84 | Aa | 57 | II | <=2 | cm | negative | no | 82.89 | rich | rich |
| al. | | | | | | | | | | | |
| Loi et | 163B27 | Aa | 49 | I | <=2 | cm | negative | no | 73.92 | rich | rich |
| al. | | | | | | | | | | | |
| Loi et | 184B38 | Aa | 63 | I | <=2 | cm | negative | no | 103.89 | rich | rich |
| al. | | | | | | | | | | | |
| Loi et | 227C50 | Aa | 57 | I | <=2 | cm | positive | yes | 108.88 | rich | poor |
| al. | | | | | | | | | | | |
| Loi et | 229C44 | Aa | 52 | I | <=2 | cm | negative | no | 113.87 | rich | poor |
| al. | | | | | | | | | | | |
| Loi et | 231C80 | Ab | 56 | I | >2 | cm | negative | yes | 76.91 | rich | poor |
| al. | | | | | | | | | | | |
| Loi et | 242C21 | Ab | 64 | II | <=2 | cm | negative | yes | 25.95 | rich | rich |
| al. | | | | | | | | | | | |
| Loi et | 247C76 | Ab | 56 | II | <=2 | cm | negative | no | 49.94 | rich | rich |
| al. | | | | | | | | | | | |
| Loi et | 248C91 | Aa | 57 | I | >2 | cm | negative | no | 34.96 | rich | rich |
| al. | | | | | | | | | | | |
| Loi et | 266C51 | Aa | 58 | I | >2 | cm | negative | no | 105.86 | rich | poor |
| al. | | | | | | | | | | | |
| Loi et | 280C43 | Aa | 45 | II | <=2 | cm | positive | yes | 11.99 | rich | rich |
| al. | | | | | | | | | | | |
| Loi et | 284C63 | Aa | 48 | I | <=2 | cm | positive | no | 112.85 | rich | rich |
| al. | | | | | | | | | | | |
| Loi et | 286C91 | Aa | 62 | II | <=2 | cm | negative | no | 87.89 | rich | rich |
| al. | | | | | | | | | | | |
| Loi et | 292C66 | Aa | 51 | II | <=2 | cm | positive | no | 107.86 | rich | rich |
| al. | | | | | | | | | | | |
| Loi et | 42C67 | Aa | 59 | I | >2 | cm | negative | no | 105.86 | rich | rich |
| al. | | | | | | | | | | | |
| Loi et | 74A63 | Ab | 56 | I | >2 | cm | negative | yes | 70.9 | rich | rich |
| al. | | | | | | | | | | | |
| vdV | 293 | Ab | 46 | I | <=2 | cm | positive | no | 76 | rich | rich |
| et al. | | | | | | | | | | | |
| vdV | 387 | Ab | 52 | II | <=2 | cm | positive | no | 99 | poor | rich |
| et al. | | | | | | | | | | | |
| vdV | 118 | Ab | 47 | II | <=2 | cm | negative | no | 63 | poor | rich |
| et al. | | | | | | | | | | | |
| vdV | 379 | Ab | 52 | I | >2 | cm | negative | no | 166 | rich | poor |
| et al. | | | | | | | | | | | |
| vdV | 146 | Ab | 47 | III | >2 | cm | positive | yes | 44 | poor | rich |
| et al. | | | | | | | | | | | |
| vdV | 264 | Aa | 42 | II | >2 | cm | positive | no | 87 | poor | poor |
| et al. | | | | | | | | | | | |
| vdV | 275 | Ab | 49 | II | >2 | cm | positive | no | 1 | rich | rich |
| et al. | | | | | | | | | | | |
| vdV | 128 | Ab | 50 | I | >2 | cm | positive | no | 105 | rich | poor |
| et al. | | | | | | | | | | | |
| vdV | 363 | Ab | 42 | II | >2 | cm | positive | yes | 60 | rich | poor |
| et al. | | | | | | | | | | | |
| vdV | 283 | Ab | 49 | III | >2 | cm | positive | no | 64 | rich | rich |
| et al. | | | | | | | | | | | |
| vdV | 349 | Ab | 45 | II | >2 | cm | negative | no | 78 | rich | rich |
| et al. | | | | | | | | | | | |
| vdV | 247 | Ab | 50 | II | <=2 | cm | positive | no | 68 | poor | rich |
| et al. | | | | | | | | | | | |
| vdV | 339 | Ab | 45 | II | <=2 | cm | negative | no | 199 | rich | poor |
| et al. | | | | | | | | | | | |
| vdV | 337 | Ab | 29 | I | <=2 | cm | positive | yes | 25 | poor | poor |
| et al. | | | | | | | | | | | |
| vdV | 348 | Ab | 50 | II | <=2 | cm | negative | no | 74 | rich | rich |
| et al. | | | | | | | | | | | |
| vdV | 159 | Ab | 44 | II | <=2 | cm | positive | yes | 53 | poor | poor |
| et al. | | | | | | | | | | | |
| vdV | 302 | Ab | 47 | III | >2 | cm | negative | no | 21 | rich | poor |
| et al. | | | | | | | | | | | |
| vdV | 322 | Ab | 45 | II | >2 | cm | positive | no | 80 | rich | poor |
| et al. | | | | | | | | | | | |
| vdV | 192 | Ab | 41 | II | <=2 | cm | positive | yes | 32 | rich | poor |
| et al. | | | | | | | | | | | |
| vdV | 107 | Ab | 38 | III | <=2 | cm | negative | yes | 31 | poor | poor |
| et al. | | | | | | | | | | | |
| vdV | 327 | Ab | 49 | II | <=2 | cm | positive | yes | 55 | poor | rich |
| et al. | | | | | | | | | | | |
| vdV | 169 | Ab | 40 | II | >2 | cm | positive | no | 179 | rich | rich |
| et al. | | | | | | | | | | | |
| vdV | 284 | Ab | 45 | II | >2 | cm | positive | yes | 47 | poor | poor |
| et al. | | | | | | | | | | | |
| vdV | 209 | Ab | 41 | I | >2 | cm | positive | yes | 79 | poor | poor |
| et al. | | | | | | | | | | | |
| vdV | 127 | Ab | 42 | I | <=2 | cm | positive | yes | 56 | poor | poor |
| et al. | | | | | | | | | | | |
| vdV | 383 | Ab | 52 | II | <=2 | cm | positive | no | 133 | poor | rich |
| et al. | | | | | | | | | | | |
| vdV | 311 | Ab | 42 | II | >2 | cm | positive | yes | 51 | poor | poor |
| et al. | | | | | | | | | | | |
| vdV | 185 | Ab | 42 | II | <=2 | cm | negative | no | 88 | rich | poor |
| et al. | | | | | | | | | | | |
| vdV | 170 | Aa | 42 | I | >2 | cm | positive | no | 160 | poor | rich |
| et al. | | | | | | | | | | | |
| vdV | 231 | Aa | 43 | II | >2 | cm | negative | yes | 43 | rich | rich |
| et al. | | | | | | | | | | | |
| vdV | 161 | Aa | 46 | I | >2 | cm | positive | yes | 98 | poor | rich |
| et al. | | | | | | | | | | | |
| vdV | 133 | Aa | 32 | I | <=2 | cm | negative | no | 104 | poor | rich |
| et al. | | | | | | | | | | | |
| vdV | 214 | Aa | 41 | I | <=2 | cm | negative | yes | 90 | rich | rich |
| et al. | | | | | | | | | | | |
| vdV | 167 | Aa | 44 | I | <=2 | cm | negative | no | 184 | rich | rich |
| et al. | | | | | | | | | | | |
| vdV | 287 | Aa | 44 | II | >2 | cm | positive | no | 73 | rich | rich |
| et al. | | | | | | | | | | | |
| vdV | 281 | Aa | 48 | II | <=2 | cm | positive | no | 88 | poor | rich |
| et al. | | | | | | | | | | | |
| vdV | 328 | Aa | 41 | I | <=2 | cm | positive | no | 67 | rich | rich |
| et al. | | | | | | | | | | | |
| vdV | 154 | Aa | 40 | I | <=2 | cm | negative | no | 181 | rich | rich |
| et al. | | | | | | | | | | | |
| vdV | 343 | Aa | 45 | I | <=2 | cm | positive | no | 79 | rich | rich |
| et al. | | | | | | | | | | | |
| vdV | 261 | Aa | 50 | I | <=2 | cm | positive | no | 103 | rich | rich |
| et al. | | | | | | | | | | | |
| vdV | 155 | Aa | 49 | III | >2 | cm | negative | yes | 11 | rich | poor |
| et al. | | | | | | | | | | | |
| vdV | 388 | Aa | 52 | II | <=2 | cm | negative | no | 87 | rich | poor |
| et al. | | | | | | | | | | | |
| vdV | 395 | Aa | 51 | II | >2 | cm | positive | yes | 135 | poor | poor |
| et al. | | | | | | | | | | | |
| vdV | 120 | Aa | 42 | II | <=2 | cm | negative | no | 121 | rich | rich |
| et al. | | | | | | | | | | | |
| vdV | 280 | Aa | 48 | I | <=2 | cm | positive | no | 64 | poor | poor |
| et al. | | | | | | | | | | | |
| vdV | 183 | Aa | 42 | I | >2 | cm | negative | no | 142 | rich | rich |
| et al. | | | | | | | | | | | |
| vdV | 123 | Aa | 48 | III | <=2 | cm | negative | no | 171 | rich | poor |
| et al. | | | | | | | | | | | |
| vdV | 125 | Aa | 50 | II | <=2 | cm | positive | no | 93 | rich | rich |
| et al. | | | | | | | | | | | |
| vdV | 14 | Aa | 48 | I | <=2 | cm | negative | no | 99 | poor | rich |
| et al. | | | | | | | | | | | |
| vdV | 315 | Aa | 40 | I | <=2 | cm | positive | no | 99 | poor | poor |
| et al. | | | | | | | | | | | |
| vdV | 191 | Aa | 34 | III | >2 | cm | negative | no | 153 | rich | rich |
| et al. | | | | | | | | | | | |
| vdV | 373 | Aa | 51 | II | >2 | cm | positive | no | 93 | poor | poor |
| et al. | | | | | | | | | | | |
| vdV | 129 | Aa | 43 | II | <=2 | cm | positive | no | 91 | poor | poor |
| et al. | | | | | | | | | | | |
| vdV | 352 | Aa | 43 | II | >2 | cm | negative | no | 70 | poor | poor |
| et al. | | | | | | | | | | | |
| vdV | 323 | Aa | 41 | I | >2 | cm | negative | no | 106 | rich | rich |
| et al. | | | | | | | | | | | |
| vdV | 6 | Aa | 49 | II | <=2 | cm | negative | no | 134 | poor | poor |
| et al. | | | | | | | | | | | |
| vdV | 271 | Aa | 42 | I | <=2 | cm | negative | no | 84 | rich | rich |
| et al. | | | | | | | | | | | |
| vdV | 122 | Aa | 43 | II | >2 | cm | negative | no | 178 | poor | poor |
| et al. | | | | | | | | | | | |
| vdV | 391 | Aa | 51 | II | >2 | cm | negative | yes | 42 | poor | poor |
| et al. | | | | | | | | | | | |
| vdV | 334 | Aa | 36 | II | >2 | cm | positive | no | 92 | poor | poor |
| et al. | | | | | | | | | | | |
| vdV | 17 | Aa | 48 | II | <=2 | cm | negative | no | 94 | poor | rich |
| et al. | | | | | | | | | | | |
| vdV | 233 | Aa | 42 | I | >2 | cm | negative | no | 169 | poor | rich |
| et al. | | | | | | | | | | | |
| vdV | 297 | Aa | 37 | II | >2 | cm | positive | no | 115 | poor | poor |
| et al. | | | | | | | | | | | |
| vdV | 303 | Aa | 43 | II | >2 | cm | positive | no | 110 | poor | poor |
| et al. | | | | | | | | | | | |
| vdV | 61 | Aa | 38 | III | <=2 | cm | negative | yes | 32 | poor | poor |
| et al. | | | | | | | | | | | |
| vdV | 145 | Aa | 48 | II | <=2 | cm | positive | no | 66 | poor | rich |
| et al. | | | | | | | | | | | |
| vdV | 9 | Aa | 48 | III | <=2 | cm | negative | no | 124 | poor | rich |
| et al. | | | | | | | | | | | |
| vdV | 358 | Aa | 45 | I | <=2 | cm | negative | no | 75 | rich | poor |
| et al. | | | | | | | | | | | |
| vdV | 157 | Aa | 45 | I | >2 | cm | positive | no | 94 | rich | rich |
| et al. | | | | | | | | | | | |
| vdV | 390 | Aa | 51 | I | <=2 | cm | positive | no | 82 | rich | poor |
| et al. | | | | | | | | | | | |
| vdV | 193 | Aa | 50 | I | <=2 | cm | negative | no | 142 | poor | poor |
| et al. | | | | | | | | | | | |
| vdV | 342 | Aa | 45 | II | <=2 | cm | negative | no | 184 | rich | rich |
| et al. | | | | | | | | | | | |
| vdV | 397 | Aa | 51 | II | >2 | cm | negative | yes | 57 | rich | poor |
| et al. | | | | | | | | | | | |
| vdV | 345 | Aa | 47 | II | >2 | cm | positive | no | 84 | poor | poor |
| et al. | | | | | | | | | | | |
| vdV | 140 | Aa | 46 | I | <=2 | cm | negative | no | 67 | poor | poor |
| et al. | | | | | | | | | | | |
| vdV | 274 | Aa | 49 | I | <=2 | cm | negative | no | 71 | rich | poor |
| et al. | | | | | | | | | | | |
| vdV | 51 | Aa | 41 | III | >2 | cm | negative | yes | 59 | rich | rich |
| et al. | | | | | | | | | | | |
| vdV | 318 | Aa | 37 | I | <=2 | cm | positive | yes | 28 | poor | poor |
| et al. | | | | | | | | | | | |
| vdV | 403 | Aa | 47 | I | >2 | cm | positive | no | 81 | poor | poor |
| et al. | | | | | | | | | | | |
| vdV | 401 | Aa | 41 | II | >2 | cm | negative | yes | 18 | rich | rich |
| et al. | | | | | | | | | | | |
| vdV | 45 | Aa | 37 | III | >2 | cm | negative | yes | 13 | rich | poor |
| et al. | | | | | | | | | | | |
| vdV | 239 | Aa | 40 | I | <=2 | cm | negative | no | 97 | poor | rich |
| et al. | | | | | | | | | | | |
| vdV | 354 | Aa | 47 | III | >2 | cm | negative | no | 74 | poor | poor |
| et al. | | | | | | | | | | | |
| vdV | 294 | Ab | 49 | II | >2 | cm | positive | no | 74 | poor | rich |
| et al. | | | | | | | | | | | |
| vdV | 305 | Ab | 40 | I | >2 | cm | negative | no | 115 | poor | rich |
| et al. | | | | | | | | | | | |
| vdV | 380 | Aa | 52 | II | <=2 | cm | negative | no | 153 | rich | rich |
| et al. | | | | | | | | | | | |
| vdV | 365 | Aa | 51 | II | <=2 | cm | negative | no | 210 | rich | rich |
| et al. | | | | | | | | | | | |
| vdV | 235 | Aa | 47 | I | <=2 | cm | negative | no | 78 | poor | poor |
| et al. | | | | | | | | | | | |
| vdV | 124 | Ab | 38 | II | <=2 | cm | negative | no | 80 | rich | rich |
| et al. | | | | | | | | | | | |
| vdV | 190 | Ab | 48 | I | <=2 | cm | positive | yes | 89 | rich | poor |
| et al. | | | | | | | | | | | |
| vdV | 56 | Ab | 30 | II | <=2 | cm | negative | yes | 56 | poor | poor |
| et al. | | | | | | | | | | | |
| vdV | 38 | Ab | 52 | II | <=2 | cm | negative | no | 88 | rich | rich |
| et al. | | | | | | | | | | | |
| vdV | 220 | Ab | 42 | I | <=2 | cm | positive | no | 124 | rich | rich |
| et al. | | | | | | | | | | | |
| vdV | 207 | Aa | 44 | I | >2 | cm | negative | no | 116 | rich | poor |
| et al. | | | | | | | | | | | |
| vdV | 290 | Ab | 49 | I | <=2 | cm | positive | no | 60 | rich | rich |
| et al. | | | | | | | | | | | |
| vdV | 126 | Ab | 38 | II | <=2 | cm | negative | yes | 76 | poor | poor |
| et al. | | | | | | | | | | | |
| vdV | 285 | Ab | 43 | II | >2 | cm | negative | no | 69 | rich | rich |
| et al. | | | | | | | | | | | |
| vdV | 188 | Aa | 41 | I | <=2 | cm | positive | no | 135 | rich | poor |
| et al. | | | | | | | | | | | |
| vdV | 295 | Aa | 48 | I | >2 | cm | negative | no | 67 | poor | rich |
| et al. | | | | | | | | | | | |

| | | | | | | | | | | |
|---|---|---|---|---|---|---|---|---|---|---|
| Wang | 130 | Ab | NA | NA | NA | negative | yes | 26 | poor | rich |
| et | | | | | | | | | | |
| al. | | | | | | | | | | |
| Wang | 203 | Ab | NA | NA | NA | negative | yes | 29 | poor | poor |
| et | | | | | | | | | | |
| al. | | | | | | | | | | |
| Wang | 863 | Ab | NA | NA | NA | negative | no | 107 | poor | poor |
| et | | | | | | | | | | |
| al. | | | | | | | | | | |
| Wang | 288 | Ab | NA | NA | NA | negative | yes | 71 | poor | poor |
| et | | | | | | | | | | |
| al. | | | | | | | | | | |
| Wang | 873 | Ab | NA | NA | NA | negative | yes | 59 | rich | poor |
| et | | | | | | | | | | |
| al. | | | | | | | | | | |
| Wang | 18 | Ab | NA | NA | NA | negative | yes | 34 | poor | poor |
| et | | | | | | | | | | |
| al. | | | | | | | | | | |
| Wang | 231 | Ab | NA | NA | NA | negative | yes | 44 | poor | poor |
| et | | | | | | | | | | |
| al. | | | | | | | | | | |
| Wang | 284 | Ab | NA | NA | NA | negative | no | 72 | rich | rich |
| et | | | | | | | | | | |
| al. | | | | | | | | | | |
| Wang | 115 | Ab | NA | NA | NA | negative | yes | 15 | rich | rich |
| et | | | | | | | | | | |
| al. | | | | | | | | | | |
| Wang | 137 | Ab | NA | NA | NA | negative | yes | 32 | poor | rich |
| et | | | | | | | | | | |
| al. | | | | | | | | | | |
| Wang | 789 | Aa | NA | NA | NA | negative | no | 96 | poor | rich |
| et | | | | | | | | | | |
| al. | | | | | | | | | | |
| Wang | 817 | Aa | NA | NA | NA | negative | no | 108 | rich | rich |
| et | | | | | | | | | | |
| al. | | | | | | | | | | |
| Wang | 290 | Aa | NA | NA | NA | negative | no | 100 | rich | rich |
| et | | | | | | | | | | |
| al. | | | | | | | | | | |
| Wang | 247 | Ab | NA | NA | NA | negative | yes | 44 | poor | poor |
| et | | | | | | | | | | |
| al. | | | | | | | | | | |
| Wang | 605 | Ab | NA | NA | NA | negative | no | 57 | rich | poor |
| et | | | | | | | | | | |
| al. | | | | | | | | | | |
| Wang | 625 | Aa | NA | NA | NA | negative | yes | 72 | poor | poor |
| et | | | | | | | | | | |
| al. | | | | | | | | | | |
| Wang | 15 | Aa | NA | NA | NA | negative | no | 99 | poor | poor |
| et | | | | | | | | | | |
| al. | | | | | | | | | | |
| Wang | 613 | Aa | NA | NA | NA | negative | no | 93 | rich | poor |
| et | | | | | | | | | | |
| al. | | | | | | | | | | |
| Wang | 747 | Aa | NA | NA | NA | negative | no | 96 | rich | poor |
| et | | | | | | | | | | |
| al. | | | | | | | | | | |
| Wang | 647 | Aa | NA | NA | NA | negative | no | 105 | poor | poor |
| et | | | | | | | | | | |
| al. | | | | | | | | | | |
| Wang | 612 | Aa | NA | NA | NA | negative | no | 92 | poor | rich |
| et | | | | | | | | | | |
| al. | | | | | | | | | | |
| Wang | 794 | Aa | NA | NA | NA | negative | no | 101 | rich | rich |
| et | | | | | | | | | | |
| al. | | | | | | | | | | |
| Wang | 778 | Aa | NA | NA | NA | negative | no | 104 | rich | rich |
| et | | | | | | | | | | |
| al. | | | | | | | | | | |
| Wang | 767 | Aa | NA | NA | NA | negative | no | 134 | poor | rich |
| et | | | | | | | | | | |
| al. | | | | | | | | | | |
| Wang | 848 | Aa | NA | NA | NA | negative | no | 86 | poor | poor |
| et | | | | | | | | | | |
| al. | | | | | | | | | | |
| Wang | 847 | Aa | NA | NA | NA | negative | no | 105 | poor | rich |
| et | | | | | | | | | | |
| al. | | | | | | | | | | |
| Wang | 253 | Aa | NA | NA | NA | negative | yes | 19 | poor | poor |
| et | | | | | | | | | | |
| al. | | | | | | | | | | |
| Wang | 785 | Aa | NA | NA | NA | negative | no | 138 | rich | poor |
| et | | | | | | | | | | |
| al. | | | | | | | | | | |
| Wang | 239 | Aa | NA | NA | NA | negative | yes | 35 | rich | poor |
| et | | | | | | | | | | |
| al. | | | | | | | | | | |
| Wang | 8 | Aa | NA | NA | NA | negative | yes | 37 | rich | rich |
| et | | | | | | | | | | |
| al. | | | | | | | | | | |
| Wang | 751 | Aa | NA | NA | NA | negative | no | 125 | rich | rich |
| et | | | | | | | | | | |
| al. | | | | | | | | | | |
| Wang | 277 | Aa | NA | NA | NA | negative | no | 79 | rich | rich |
| et | | | | | | | | | | |
| al. | | | | | | | | | | |
| Wang | 913 | Aa | NA | NA | NA | negative | yes | 80 | rich | poor |
| et | | | | | | | | | | |
| al. | | | | | | | | | | |
| Wang | 244 | Aa | NA | NA | NA | negative | yes | 39 | rich | rich |
| et | | | | | | | | | | |
| al. | | | | | | | | | | |
| Wang | 769 | Aa | NA | NA | NA | negative | no | 84 | rich | poor |
| et | | | | | | | | | | |
| al. | | | | | | | | | | |
| Wang | 874 | Aa | NA | NA | NA | negative | yes | 70 | rich | poor |
| et | | | | | | | | | | |
| al. | | | | | | | | | | |
| Wang | 868 | Aa | NA | NA | NA | negative | yes | 77 | poor | poor |
| et | | | | | | | | | | |
| al. | | | | | | | | | | |
| Wang | 82 | Aa | NA | NA | NA | negative | no | 143 | rich | rich |
| et | | | | | | | | | | |
| al. | | | | | | | | | | |
| Wang | 28 | Aa | NA | NA | NA | negative | no | 155 | poor | rich |
| et | | | | | | | | | | |
| al. | | | | | | | | | | |
| Wang | 601 | Aa | NA | NA | NA | negative | no | 52 | poor | rich |
| et | | | | | | | | | | |
| al. | | | | | | | | | | |
| Wang | 815 | Aa | NA | NA | NA | negative | no | 107 | rich | rich |
| et | | | | | | | | | | |
| al. | | | | | | | | | | |
| Wang | 634 | Aa | NA | NA | NA | negative | no | 117 | rich | poor |
| et | | | | | | | | | | |
| al. | | | | | | | | | | |
| Wang | 798 | Aa | NA | NA | NA | negative | no | 132 | poor | rich |
| et | | | | | | | | | | |
| al. | | | | | | | | | | |
| Wang | 272 | Aa | NA | NA | NA | negative | no | 83 | rich | poor |
| et | | | | | | | | | | |
| al. | | | | | | | | | | |
| Wang | 614 | Aa | NA | NA | NA | negative | no | 88 | poor | rich |
| et | | | | | | | | | | |
| al. | | | | | | | | | | |
| Wang | 89 | Aa | NA | NA | NA | negative | yes | 2 | poor | poor |
| et | | | | | | | | | | |
| al. | | | | | | | | | | |
| Wang | 762 | Aa | NA | NA | NA | negative | no | 116 | poor | poor |
| et | | | | | | | | | | |
| al. | | | | | | | | | | |
| Wang | 779 | Aa | NA | NA | NA | negative | no | 137 | poor | rich |
| et | | | | | | | | | | |
| al. | | | | | | | | | | |
| Wang | 737 | Aa | NA | NA | NA | negative | no | 123 | rich | rich |
| et | | | | | | | | | | |
| al. | | | | | | | | | | |
| Wang | 635 | Aa | NA | NA | NA | negative | no | 119 | rich | poor |
| et | | | | | | | | | | |
| al. | | | | | | | | | | |
| Wang | 783 | Aa | NA | NA | NA | negative | no | 122 | rich | rich |
| et | | | | | | | | | | |
| al. | | | | | | | | | | |
| Wang | 716 | Aa | NA | NA | NA | negative | no | 87 | poor | poor |
| et | | | | | | | | | | |
| al. | | | | | | | | | | |
| Wang | 286 | Aa | NA | NA | NA | negative | no | 107 | poor | rich |
| et | | | | | | | | | | |
| al. | | | | | | | | | | |
| Wang | 32 | Aa | NA | NA | NA | negative | no | 84 | poor | rich |
| et | | | | | | | | | | |
| al. | | | | | | | | | | |
| Wang | 40 | Aa | NA | NA | NA | negative | no | 102 | rich | rich |
| et | | | | | | | | | | |
| al. | | | | | | | | | | |
| Wang | 795 | Aa | NA | NA | NA | negative | no | 132 | poor | rich |
| et | | | | | | | | | | |
| al. | | | | | | | | | | |
| Wang | 851 | Aa | NA | NA | NA | negative | no | 92 | poor | rich |
| et | | | | | | | | | | |
| al. | | | | | | | | | | |
| Wang | 275 | Aa | NA | NA | NA | negative | no | 105 | poor | poor |
| et | | | | | | | | | | |
| al. | | | | | | | | | | |
| Wang | 122 | Aa | NA | NA | NA | negative | no | 104 | poor | rich |
| et | | | | | | | | | | |
| al. | | | | | | | | | | |
| Wang | 642 | Aa | NA | NA | NA | negative | no | 54 | rich | rich |
| et | | | | | | | | | | |
| al. | | | | | | | | | | |
| Wang | 754 | Aa | NA | NA | NA | negative | no | 109 | rich | poor |
| et | | | | | | | | | | |
| al. | | | | | | | | | | |
| Wang | 870 | Aa | NA | NA | NA | negative | yes | 56 | poor | rich |
| et | | | | | | | | | | |
| al. | | | | | | | | | | |
| Wang | 254 | Aa | NA | NA | NA | negative | yes | 48 | poor | poor |
| et | | | | | | | | | | |
| al. | | | | | | | | | | |
| Wang | 808 | Aa | NA | NA | NA | negative | no | 110 | rich | poor |
| et | | | | | | | | | | |
| al. | | | | | | | | | | |
| Wang | 631 | Aa | NA | NA | NA | negative | no | 99 | poor | rich |
| et | | | | | | | | | | |
| al. | | | | | | | | | | |
| Wang | 240 | Aa | NA | NA | NA | negative | yes | 36 | poor | poor |
| et | | | | | | | | | | |
| al. | | | | | | | | | | |
| Wang | 234 | Aa | NA | NA | NA | negative | yes | 37 | rich | poor |
| et | | | | | | | | | | |
| al. | | | | | | | | | | |
| Wang | 141 | Aa | NA | NA | NA | negative | yes | 25 | rich | rich |
| et | | | | | | | | | | |
| al. | | | | | | | | | | |
| Wang | 138 | Aa | NA | NA | NA | negative | yes | 47 | rich | poor |
| et | | | | | | | | | | |
| al. | | | | | | | | | | |
| Wang | 287 | Aa | NA | NA | NA | negative | no | 79 | poor | rich |
| et | | | | | | | | | | |
| al. | | | | | | | | | | |
| Wang | 876 | Aa | NA | NA | NA | negative | yes | 60 | poor | rich |
| et | | | | | | | | | | |
| al. | | | | | | | | | | |
| Wang | 728 | Aa | NA | NA | NA | negative | no | 105 | rich | poor |
| et | | | | | | | | | | |
| al. | | | | | | | | | | |
| Wang | 201 | Aa | NA | NA | NA | negative | no | 113 | rich | poor |
| et | | | | | | | | | | |
| al. | | | | | | | | | | |
| Wang | 134 | Aa | NA | NA | NA | negative | yes | 28 | rich | rich |
| et | | | | | | | | | | |
| al. | | | | | | | | | | |
| Wang | 99 | Aa | NA | NA | NA | negative | no | 107 | rich | rich |
| et | | | | | | | | | | |
| al. | | | | | | | | | | |
| Wang | 760 | Aa | NA | NA | NA | negative | no | 98 | poor | poor |
| et | | | | | | | | | | |
| al. | | | | | | | | | | |
| Wang | 222 | Aa | NA | NA | NA | negative | yes | 37 | rich | poor |
| et | | | | | | | | | | |
| al. | | | | | | | | | | |
| Wang | 200 | Aa | NA | NA | NA | negative | no | 108 | rich | rich |
| et | | | | | | | | | | |
| al. | | | | | | | | | | |
| Wang | 741 | Aa | NA | NA | NA | negative | no | 124 | rich | poor |
| et | | | | | | | | | | |
| al. | | | | | | | | | | |

In supplementary table 3, Loi et al. refers to Loi S, Haibe-Kains B, Desmedt C, et al. Definition of clinically distinct molecular subtypes in estrogen receptor-positive breast carcinomas through genomic grade. J Clin Oncol 2007; 25: 1239-46 [16], vdV et al. refers to Van de Vijver MJ, He YD, van't Veer LJ, et al. A gene-expression signature as a predictor of survival in breast cancer. N Engl J Med 2002; 347: 1999-2009 [14], and Wand et al. refers to Wang Y, Klijn JG, Zhang Y, et al. Gene-expression profiles to predict distant metastasis of lymph-node-negative primary breast cancer. Lancet 2005; 365: 671-9 [15].

### Statistical Analyses

We defined a score, called the Kinase Score (KS), which was based on the expression level of 16 kinase genes. It was defined as:

$$KS = \frac{A}{n} {\sum }_{i = 1}^{n} ( xi - B )$$

where A and B represent normalization parameters, which make the KS comparable across the different datasets, n the number of available kinase genes (7 to 16), and xi the logarithmic gene expression level in tumor i. Using a cut-off value of 0, each tumor was assigned a low score (KS<0, i.e. with overall low expression of 16 kinase genes) or a high score (KS>0, i.e. with overall strong expression of 16 kinase genes). In the present invention, the number of available kinase genes, i.e. n, is from 1 to 16.

The samples included in the statistical analysis (luminal A subtype) were ER and/or PR-positive as defined using immunohistochemistry (IHC). We introduced two qualitative variables based on the mRNA expression level of ER and PR (ESR1 estrogen receptor 1 probe set 205225_at and PGR progesterone receptor probe set 208305_at): the cut-off for defining ESR1 or PGR-rich or -poor was the median expression level of the corresponding probe set. The two probe sets were chosen by using the same above-cited criteria.

Correlations between sample groups and histoclinical factors were calculated with the Fisher's exact test for qualitative variables with discrete categories, and the Wilcoxon test for continuous variables. Follow-up was measured from the date of diagnosis to the date of last news for patients without relapse. Relapse-free survival (RFS) was calculated from the date of diagnosis until date of first relapse whatever its location (local, regional or distant) using the Kaplan-Meier method and compared between groups with the log-rank test. The univariate and multivariate analyses were done using Cox regression analysis. The p-values were based on log-rank test, and patients with one or more missing data were excluded. All statistical tests were two-sided at the 5% level of significance. Statistical analysis was done using the survival package (version 2.30), in the R software (version 2.4.1—www.cran.r-project.org).

### Results

### Gene Expression Profiling of Breast Cancer and Molecular Subtypes

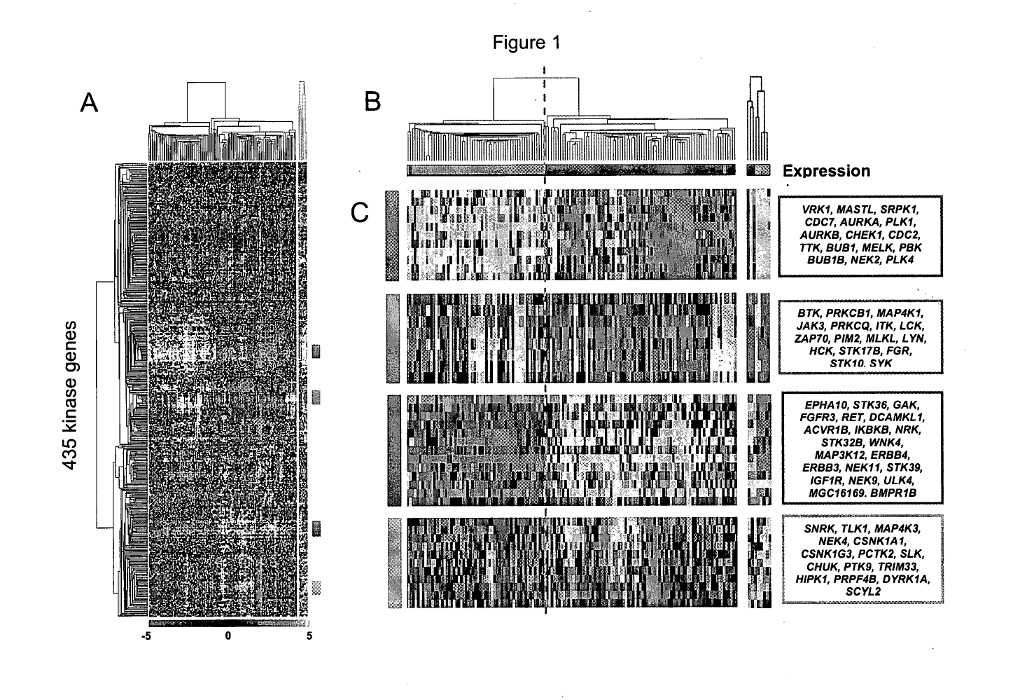

A total of 227 samples were profiled using whole-genome DNA microarrays. Hierarchical clustering was applied to the 14,486 genes/ESTs with significant variation in expression level across all samples (Supplementary FIG. 1). Clusters of samples and clusters of genes were identified, and represented previously recognized groups (Bertucci F, Finetti P, Cervera N, et al. Gene expression profiling shows medullary breast cancer is a subgroup of basal breast cancers. Cancer Res 2006; 66:4636-44 [17]). We looked whether the five molecular subtypes reported by others [2-4] were also present in our series of samples by using the 476 genes common to the intrinsic 500-gene set. We had previously shown that clustering of the available RNA expression data for these 476 genes in the 122 samples from Sorlie et al discriminated the same five molecular subtypes [17], allowing the definition of typical expression profile of each subtype for our gene set (thereafter designated centroid) with 96% of concordance with those defined on the whole intrinsic gene set. We measured the Pearson correlation of each of our 227 tissue samples with each centroid. The highest coefficient defined the subtype, with a minimum threshold of 0.15. Subtypes are color-coded in Supplementary FIG. 1: they included 91 luminal A samples, and 67 basal samples, as well as other subtypes.

### Whole Kinome Expression Profiling Separates Basal and Luminal A Breast Cancers

We wanted to identify kinase genes whose differential expression is associated with clinical outcome. We focused our analysis on two major subtypes of BC with opposite prognosis, the basal and the luminal A subtypes. From our subtyping, we selected a series of 138 BC samples with available full histoclinical annotations, including 80 luminal A and 58 basal BCs. We identified a total of 435 unique Affymetrix probe sets for 435 kinases as satisfying simultaneously presence, quality and reliability (Supplementary Table 4).

SUPPLEMENTARY TABLE 4
Distribution of the molecular subtypes of tumors and number
of the 16 mitotic in the three published expression data sets

| | | No. genes | | | | | | | | |
|---|---|---|---|---|---|---|---|---|---|---|
| | | common to the | | | | | | | | No. kinases |
| | | intrinsic set of | | | | | | | | common to the 16 |
| | No. | Sorlie and | | | | | | | Concordance of | kinase gene set and |
| Data set | Tumors | expression data | Basal | Luminal A | Luminal B | ERBB2 | Normal | NA* | the centroids | expression data** |
| Wang et | 286 | 432 | 58 | 79 | 27 | 38 | 33 | 51 | 90% | 15 (22) |
| al. | | | | | | | | | | |
| van de | | | | | | | | | | |
| Vijver et | 295 | 406 | 46 | 99 | 24 | 49 | 28 | 49 | 91% | 7 (7) |
| al. | | | | | | | | | | |
| Loi et al. | 414 | 472 | 43 | 98 | 46 | 54 | 94 | 79 | 94% | 16 (26) |

*Numbers of tumors without any assigned subtype
**Numbers in parentheses are numbers of all corresponding probe sets

A hierarchical clustering analysis was applied to these probe sets and 138 BCs and 8 cell lines (FIG. 1A). The tumors displayed heterogeneous expression profiles. They were sorted into two large clusters, which nearly perfectly correlated with the molecular subtype, with all but one of the basal BCs in the left cluster and all but one of the luminal A BCs in the right cluster (FIG. 1B). Visual inspection revealed at least four clusters of related genes responsible for much of the subdivision of samples into two main groups. They are zoomed in FIG. 1C. The first cluster was enriched in genes involved in cell cycle and mitosis. It was overexpressed in basal overall as compared with luminal A tumors, and in cell lines as compared with cancer tissue samples. The second gene cluster included many genes involved in immune reactions. It was expressed at heterogeneous levels in both luminal A and basal tumors, and was overexpressed in lymphocytic cell lines as compared to epithelial cell lines. The third and the fourth clusters were strongly overexpressed in luminal A overall as compared with basal BC samples. The third cluster included genes involved in TGFβ signaling as well as transmembrane tyrosine kinase receptors. Gene ontology analysis using Ingenuity software (Ingenuity Pathway Analysis v5, www.ingenuity.com) confirmed these data with significant overrepresentation (right-tailed Fisher's exact test) of the functions “cell cycle” (p=4.6E-07) and “DNA replication, recombination, and repair” (p=6.1E-05) in the first cluster, “immune response” (p=8.1E-10) and “cellular growth and proliferation” (p=8.1E-10) in the second cluster, “tumor morphology” (p=2.2E-04) and “nervous system development and function” (p=2.3E-04) in the third cluster. Analysis of canonical pathways showed overrepresentation of “G2/M transition of the cell cycle” (p=6.8E-08) “NFKB (Nuclar Factor Kappa-B) signaling pathway” (p=1.3E-04) and “TGFβ (Tumor Growth Factor Beta) signaling” (p=4E-03) in the first, second and third clusters, respectively. No correlation was found between these gene clusters and the nine kinase families (AGC (Cyclic nucleotide regulated protein kinase and close relatives family), CAMK (Kinases regulated by Ca²⁺/CaM and close relatives family), CK1 (Cyclin kinase), CMGC (Cyclin-dependent kinases (CDKs) and close relatives family), RGC (receptor guanylate cyclases), STE (protein kinases involved in MAP kinase cascades), TK (Tyrosine kinase and close relatives family), TKL (tyrosine kinase related to Ick-lymphocyte-specific protein tyrosine kinase-), and Atypical) or the chromosomal location of genes.

These results suggest that kinase gene expression is highly different between basal and luminal A BCs.

### Kinase Gene Expression Identifies Two Subgroups of Luminal A Breast Cancers

As shown in FIG. 1, basal BCs constituted a rather homogenous cluster whereas luminal A BCs were more heterogenous. Basal and luminal BCs were distinguished by the differential expression of clusters of genes. By using QT clustering, we identified a single cluster of significance principally responsible for this discrimination (FIG. 1B), corresponding to the above-described first cluster. It contained 16 kinase genes (Table 1), which were overexpressed in all basal BCs and some luminal A samples, and underexpressed in most luminal A samples (FIG. 1B).

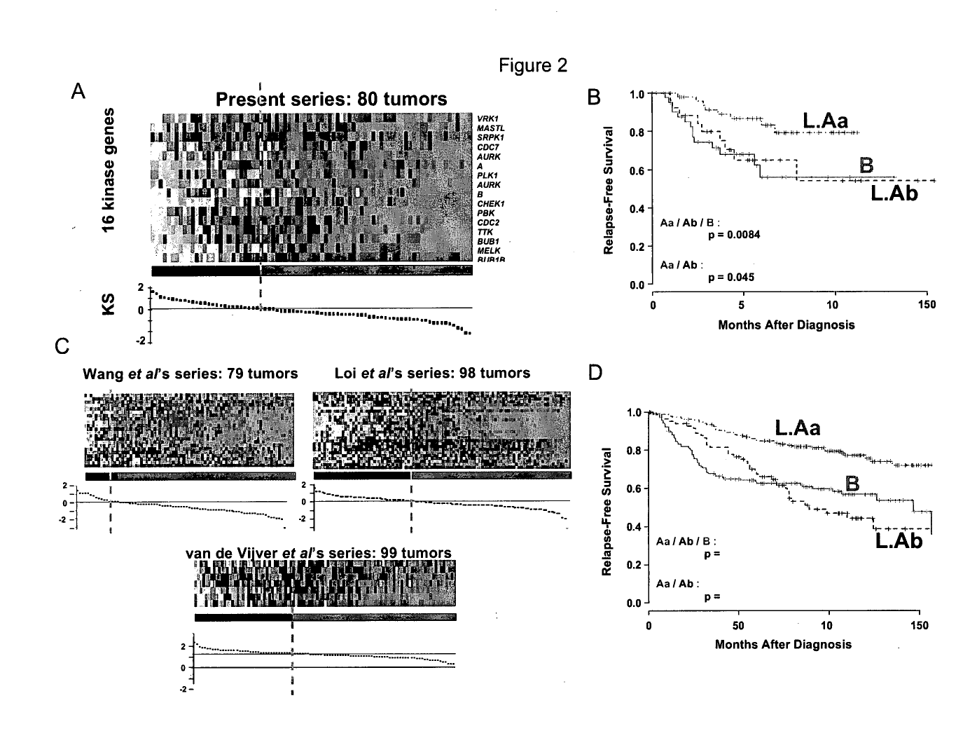

This subdivision of luminal A tumors led us to define for each of them the Kinase Score (KS) based on expression level of these 16 genes. A cut-off of 0 identified two tumor groups: a group containing the luminal A BCs with negative score (hereafter designated Aa) and a group containing the luminal A BCs with positive score (hereafter designated Ab; FIG. 2A). Luminal Aa made up two-thirds of the luminal A cases and luminal Ab BCs the remaining one-third.

Proteins encoded by the 16 genes overexpressed in luminal Ab BCs (Table 1) are all serine/threonine kinases (except SRPK1, which is a serine/arginine kinase) involved in the regulation of the late phases of the cell cycle, suggesting that luminal Ab tumors show a transcriptional program associated with mitosis.

### Characteristics and Prognosis of the Two Subgroups of Luminal A Breast Cancers

The histoclinical characteristics of the two luminal A subgroups are listed in Table 3. Strikingly, they shared most features but were different according to SBR grade with more grade III in the Ab subgroup and more grade I-II in the Aa subgroup. Ki67 expression did not distinguish Ab from Aa cases but three-fourths of luminal Ab were Ki67-positive. In conclusion, no factor but grade could distinguish Aa from Ab BCs.

TABLE 3
Histoclinical characteristics of the two luminal A tumor subgroups

| | No. Luminal A tumors (percent of evaluated cases) | | | | | | |
|---|---|---|---|---|---|---|---|
| | Total | | Luminal Aa subgroup | | Luminal Ab subgroup | | |
| Characteristics* | (N = 80) | | (N = 53) | | (N = 27) | | p** |
| Age (years) | | | | | | | 0.64 |
| Median | 56 | (24-82) | 56 | (28-82) | 55 | (24-82) | |
| (range) | | | | | | | |
| Pathological | | | | | | | 0.28 |
| type (80) | | | | | | | |
| CAN | 65 | (81%) | 41 | (77%) | 24 | (89%) | |
| MIX | 6 | (8%) | 5 | (9%) | 1 | (4%) | |
| LOB | 9 | (1%) | 7 | (14%) | 2 | (7%) | |
| Pathological | | | | | | | 1 |
| tumor size | | | | | | | |
| (69) | | | | | | | |
| >2 cm | 52 | (66%) | 34 | (76%) | 18 | (75%) | |
| ≦2 cm | 17 | (33%) | 11 | (24%) | 6 | (25%) | |
| SBR grade | | | | | | | 150E−06 |
| (79) | | | | | | | |
| I-II | 50 | (63%) | 41 | (79%) | 9 | (33%) | |
| III | 29 | (37%) | 11 | (21%) | 18 | (67%) | |
| Pathological | | | | | | | 0.8 |
| axillary | | | | | | | |
| lymph node | | | | | | | |
| status (76) | | | | | | | |
| Positive | 53 | (66%) | 35 | (66%) | 18 | (66%) | |
| Negative | 23 | (33%) | 14 | (33%) | 9 | (33%) | |
| IHC ER | | | | | | | 0.089 |
| status (80) | | | | | | | |
| Positive | 73 | (91%) | 46 | (87%) | 27 | (100%) | |
| Negative | 7 | (9%) | 7 | (13%) | 0 | (0%) | |
| IHC PR | | | | | | | 0.27 |
| status (80) | | | | | | | |
| Positive | 62 | (78%) | 39 | (74%) | 23 | (85%) | |
| Negative | 18 | (22%) | 14 | (26%) | 4 | (15%) | |
| IHC P53 | | | | | | | 1 |
| status (73) | | | | | | | |
| Positive | 15 | (21%) | 10 | (22%) | 5 | (19%) | |
| Negative | 58 | (79%) | 36 | (78%) | 22 | (81%) | |
| IHC | | | | | | | 0.327 |
| Ki67/MIB1 | | | | | | | |
| status (76) | | | | | | | |
| Positive | 47 | (62%) | 28 | (57%) | 19 | (72%) | |
| Negative | 29 | (38%) | 21 | (43%) | 8 | (28%) | |
| IHC ERBB2 | | | | | | | 0.329 |
| status (80) | | | | | | | |
| Positive | 4 | (4%) | 2 | (4%) | 3 | (11%) | |
| Negative | 76 | (96%) | 51 | (96%) | 24 | (89%) | |
| ESR1 | | | | | | | 0.238 |
| mRNA level | | | | | | | |
| (80) | | | | | | | |
| rich | 42 | (53%) | 25 | (47%) | 17 | (63%) | |
| poor | 38 | (47%) | 28 | (53%) | 10 | (37%) | |
| PGR mRNA | | | | | | | 0.641 |
| level (80) | | | | | | | |
| rich | 41 | (51%) | 26 | (48%) | 15 | (56%) | |
| poor | 39 | (49%) | 27 | (52%) | 12 | (44%) | |
| Relapse | | | | | | | 0.083 |
| (80) | | | | | | | |
| Yes | 17 | (21%) | 8 | (15%) | 9 | (33%) | |
| No | 63 | (79%) | 45 | (85%) | 18 | (67%) | |

| | | | | |
|---|---|---|---|---|
| 5-years | 76% | 83% | 65% | 0.045 |
| RFS (80) | | | | |

*In parentheses are numbers of evaluated cases among 80 tumors.
**To assess differences in clinicopathologic features between the two groups of Luminal A patients, Fisher's Exact test was used for qualitative variables with discrete categories, the Wilcoxon test was used for continuous variables, and the log-rank test was used to compare Kaplan-Meier RFS.

We compared the survival of three groups of patients, i.e. patients with basal, luminal Aa and luminal Ab BCs. We excluded from analysis the basal medullary breast cancers known to harbor good prognosis. With a median follow-up of 55 months after diagnosis, 5-year relapse-free survival (RFS; FIG. 2B) was best for patients with luminal Aa tumors (53 samples, 83% RFS), and worse for patients with luminal Ab tumors (27 samples, 65% RFS) and for patients with basal BC (43 samples, 62% RFS; p=0.031, log-rank test). Thus, the expression of 16 kinase genes (KG set) identified within luminal A tumors of apparent good prognosis a subgroup that showed a prognosis similar to basal cases.

We then compared the prognostic ability of our KS-based classifier with other histoclinical factors (age, pathological tumor size, SBR grade, and axillary lymph node status, IHC P53 (1%) and Ki67 (20%) status, ESR1 and PGR mRNA levels) in our 80 luminal A samples (Table 4A). In univariate and multivariate Cox analyses, the only factor that correlated with RFS was the KS-based classifier. The hazard ratio (HR) for relapse was 7.77 for luminal Ab tumors compared to luminal Aa tumors ([95% CI 1.97-30.66], p=0.003).

### Validation of Two Prognostic Subgroups of Luminal A Breast Cancers in Published Series

As a validation step, we analyzed three sets of published gene expression data to identify and compare the two subgroups of luminal A BCs identified by the KS. We first defined as above the molecular subtypes of tumors. Before assigning a subtype, each centroid was evaluated by its concordance with those defined by Sorlie et al [4], and none was under 90% in the three data sets. The distribution of the subtypes is shown in Supplementary Table 5.

SUPPLEMENTARY TABLE 5
Histoclinical characteristics of the two luminal A tumor subgroups and
the luminal B subtype in the three published expression data sets
Loi & van de Vijver data sets

| | No. Luminal A tumors | | | | | | |
|---|---|---|---|---|---|---|---|
| | (percent of evaluated cases) | | | | | | |
| | Luminal Aa | Luminal Ab | | | | | |
| | subgroup | subgroup | | | L. B vs L. Aa | L. B vs L. Ab | 3k |
| Characteristics* | (N = 123) | (N = 74) | p** | Lu. B | p** | p** | p** |
| Age (years) | | | 0.84 | | | | |
| Median | 51 (32-79) | 52 (29-84) | | 55 (36-86) | 0.167784744 | 0.421156552 | — |
| (range) | | | | | | | |
| Pathological | | | 0.1365 | | | | |
| tumor Size | | | | | | | |
| (195) | | | | | | | |
| >2 cm | 49 (40%) | 38 (52%) | | 44 (64%) | 247E−05 | 0.1767 | 638E−05 |
| ≦2 cm | 73 (60%) | 35 (48%) | | 25 (36%) | | | |
| SBR grade | | | 494E−04 | | | | |
| (175) | | | | | | | |
| I + II | 51 (46%) | 18 (28%) | | 32 (49%) | 3.16e−11 | 6.186e−06 | 1.741e−11 |
| III | 12 (11%) | 10 (15%) | | 33 (51%) | | | |
| Pathological | | | 0.07251 | | | | |
| axillary | | | | | | | |
| lymph node | | | | | | | |
| status (194) | | | | | | | |
| Positive | 46 (38%) | 38 (52%) | | 29 (43%) | 0.535 | 0.3149 | 0.1626 |
| Negative | 75 (62%) | 35 (48%) | | 38 (57%) | | | |
| ESR1 | | | 1 | | | | |
| mRNA level | | | | | | | |
| (197) | | | | | | | |
| rich | 87 (71%) | 52 (70%) | | 35 (50%) | 519E−05 | 169E−04 | 102E−04 |
| poor | 36 (29%) | 22 (30%) | | 35 (50%) | | | |
| PGR mRNA | | | 0.7626 | | | | |
| level (197) | | | | | | | |
| rich | 77 (63%) | 44 (59%) | | 27 (39%) | 159E−05 | 133E−04 | 411E−05 |
| poor | 46 (37%) | 30 (41%) | | 43 (61%) | | | |
| Relapse | | | 497E−06 | | | | |
| (195) | | | | | | | |
| yes | 23 (19%) | 31 (42%) | | 38 (55%) | 403E−09 | 0.1789 | 5.805E−07 |
| No | 99 (81%) | 42 (58%) | | 31 (45%) | | | |
| 5-years | | | 230E−07 | | | | |
| relapse | 89% | 75% | | 50% | 463E−10 | 0.12 | 924E−11 |
| (195) | | | | | | | |

Wang data set

| | No. Luminal A tumors | | | | | | |
|---|---|---|---|---|---|---|---|
| | (percent of evaluated cases) | | | | | | |
| | Luminal Aa | Luminal Ab | | | | | |
| | subgroup | subgroup | | | L. B vs L. Aa | L. B vs L. Ab | 3k |
| Characteristics* | (N = 67) | (N = 12) | p** | Lu. B | p** | p** | p** |
| ESR1 | | | 0.2247 | 7 (26%) | 214E−04 | 0.7086 | 355E−04 |
| mRNA level | | | | | | | |
| (79) | | | | | | | |
| rich | 36 (54%) | 4 (33%) | | 20 (74%) | | | |
| poor | 31 (46%) | 8 (67%) | | | | | |
| PGR mRNA | | | 0.2247 | 8 (30%) | 414E−04 | 1 | 0.07255 |
| level (79) | | | | | | | |
| rich | 36 (54%) | 4 (33%) | | 19 (70%) | | | |
| poor | 31 (46%) | 8 (67%) | | | | | |
| Relapse | | | 226E−05 | 13 (48%) | 0.05588 | 0.1685 | 324E−05 |
| (79) | | | | | | | |
| yes | 18 (27%) | 9 (75%) | | 14 (52%) | | | |
| No | 49 (73%) | 3 (25%) | | | | | |
| 5-years | | | 336E−07 | | | | |
| relapse (79) | 79% | 31% | | 52% | 100E−04 | 0.24 | 843E−07 |

*In parentheses are numbers of evaluated cases among 80 tumors.
**To assess differences in clinicopathologic features between the two groups of Luminal A patients, Fisher's Exact test was used for qualitative variables with discrete categories and the Wilcoxon test was used for continuous variables. Five years relapse was done using the Kaplan-Meier method and compared between groups with the log-rank test.

A total of 276 samples were identified as luminal A. The number of genes in the KG set represented in each dataset ranged from 7 to 16 (Supplementary Table 5). We computed the KS for each tumor. The same cut-off as in our series led to the identification of Aa (190 samples) and Ab (86 samples) subgroups in each set (FIG. 2C), with the same proportions as in our own series.

Samples form the three studies were pooled before prognostic analyses. Histoclinical correlations of the two subgroups were similar to those found in our series (Supplementary Table 6).

SUPPLEMENTARY TABLE 6
RFS in published series

| | | | Luminal Aa | | Luminal Ab | | RFS |
|---|---|---|---|---|---|---|---|
| | Type DNA | | | 5-years | | 5-years | probability |
| Series | Chip | * Kinase | N = | RFS | N = | RFS | **P |
| van de Vijver et | Agilent - 22,000 | 7 (7) | 62 | 87% | 37 | 66% | 0.0144 |
| al. NEJM 2002 | oligo. | | | | | | |
| Wang et al. | Affymetrix - | 15 (22) | 69 | 78% | 10 | 30% | 2.3E−05 |
| Lancet 2005 | 22,000 oligo. | | | | | | |
| Sotiriou et al. | Affymetrix - | 15 (23) | 33 | 97% | 21 | 70% | 0.00437 |
| JNCI 2006 | 22,000 oligo. | | | | | | |
| Loi S. et al. JCO | Affymetrix - | 16 (26) | 54 | 77% | 38 | 74% | 0.297 |
| 2007 | 22,000 oligo. | | | | | | |

Numbers in parentheses are numbers of total probe sets/clones.
**Log-rank p-value. Log-rank tests were used to assess the differences in both groups of LuminalA.

We then compared RFS of the two luminal A subgroups in the 276 samples. With a median follow-up of 104 months after diagnosis, luminal Ab tumors were associated with a worse prognosis than luminal Aa tumors, with respective 5-year RFS of 90% and 73% (p=6.3E-6, log-rank test; FIG. 2D). For comparison, 5-year RFS was 64% in basal samples in the three pooled series.

We also performed univariate and multivariate survival analyses (Table 4B). Wang et al's series (79 Luminal A samples) was analyzed separately due to the lack of available histoclinical data. In univariate analysis, the HR for relapse was 4.84 for luminal Ab tumors compared to luminal Aa tumors ([95% CI 2.13-11.00], p=1.7E-04). The two other series were merged for analyses (197 Luminal A samples). Three variables, including pathological tumor size, PGR mRNA expression level and KS-based subgrouping, were significantly associated to RFS in univariate analysis. In multivariate analysis, only the KS-based classifier retained significant prognostic value, confirming the prominence of the KS over the SBR grade and other variables. The HR for relapse was 2.48 for luminal Ab tumors compared to luminal Aa tumors ([95% CI 1.37-4.50], p=0.002)

TABLE 4
Univariate and multivariate RFS analyses by Cox regression of
luminal A tumors. A: in our series. B: in published series.
A. Univariate and multivariate RFS analyses by Cox regression of 80
luminal A tumors

| | Univariate Analysis | | | | Multivariate Analysis | | | |
|---|---|---|---|---|---|---|---|---|
| | | Hazard | | | | Hazard | | |
| Variables | N* | Ratio | 95% CI | p | N* | Ratio | 95% CI | p |
| This study | | | | | | | | |
| Age >50 years | 80 | 3.08 | 0.88 to | 0.08 | 64 | 5.09 | 0.72 to | 0.1 |
| (vs ≦50 years) | | | 10.8 | | | | 35.57 | |
| Pathological | 69 | 1.9 | 0.54 to | 0.32 | 64 | 4.77 | 0.86 to | 0.07 |
| tumor size | | | 6.75 | | | | 26.41 | |
| >2 cm (vs ≦2 cm) | | | | | | | | |
| SBR grade III | 79 | 1.71 | 0.66 to | 0.27 | 64 | 1.62 | 0.43 to | 0.47 |
| (vs I + II) | | | 4.46 | | | | 6.03 | |
| Pathological | 80 | 1.57 | 0.51 to | 0.43 | 64 | 1.43 | 0.32 to | 0.63 |
| axillary lymph | | | 4.82 | | | | 6.24 | |
| node status | | | | | | | | |
| positive (vs | | | | | | | | |
| negative) | | | | | | | | |
| IHC P53 status | 73 | 1.65 | 0.52 to | 0.4 | 64 | 1.62 | 0.37 to | 0.52 |
| positive (vs | | | 5.27 | | | | 7.01 | |
| negative) | | | | | | | | |
| IHC Ki67/MIB1 | 76 | 1.13 | 0.4 to | 0.82 | 64 | 0.52 | 0.12 to | 0.37 |
| status positive | | | 3.17 | | | | 2.18 | |
| (vs negative) | | | | | | | | |
| ESR1 mRNA | 80 | 2.09 | 0.73 to | 0.17 | 64 | 1.12 | 0.2 to 6.27 | 0.9 |
| rich (vs poor) | | | 5.94 | | | | | |
| PGR mRNA | 80 | 0.64 | 0.24 to | 0.36 | 64 | 0.23 | 0.05 to | 0.06 |
| rich (vs poor) | | | 1.68 | | | | 1.06 | |
| KG subgroups | 80 | 2.57 | 0.99 to | 500E−04 | 64 | 7.77 | 1.97 to | 340E−05 |
| L. Ab (vs L. Aa) | | | 6.68 | | | | 30.66 | |

TABLE 4B
Univariate and multivariate analyses by Cox regression of luminal A
tumors from published datasets

| | Univariate Analysis | | | | Multivariate Analysis | | | |
|---|---|---|---|---|---|---|---|---|
| | | Hazard | | | | Hazard | | |
| Variables | N* | Ratio | 95% CI | p | N* | Ratio | 95% CI | p |
| Loi & van | | | | | | | | |
| de Vijver | | | | | | | | |
| data sets | | | | | | | | |
| Age >50 | 195 | 1.03 | 0.57 to | 0.91 | 173 | 0.98 | 0.53 | 0.94 |
| years (vs ≦50 | | | 1.66 | | | | to | |
| years) | | | | | | | 1.81 | |
| Pathological | 195 | 2.04 | 1.19 to | 980E−05 | 173 | 1.6 | 0.89 | 0.12 |
| tumor size | | | 3.5 | | | | to | |
| >2 cm (vs | | | | | | | 2.87 | |
| ≦2 cm) | | | | | | | | |
| SBR grade | 175 | 1.6 | 0.77 to | 0.2 | 173 | 1.58 | 0.72 | 0.26 |
| III (vs I + II) | | | 3.31 | | | | to | |
| | | | | | | | 3.47 | |
| Pathological | 192 | 1.56 | 0.91 to | 0.11 | 173 | 1.4 | 0.76 | 0.28 |
| axillary | | | 2.67 | | | | to | |
| lymph node | | | | | | | 2.57 | |
| status | | | | | | | | |
| positive (vs | | | | | | | | |
| negative) | | | | | | | | |
| ESR1 | 195 | 0.67 | 0.38 to | 0.17 | 173 | 0.8 | 0.42 | 0.49 |
| mRNA rich | | | 1.18 | | | | to | |
| (vs poor) | | | | | | | 1.51 | |
| PGR mRNA | 195 | 0.44 | 0.26 to | 300E−05 | 173 | 0.56 | 0.31 | 0.051 |
| rich (vs | | | 0.76 | | | | to | |
| poor) | | | | | | | 1.00 | |
| KG | 195 | 3.07 | 1.78 to | 550E−07 | 173 | 2.48 | 1.37 | 290E−05 |
| subgroups | | | 5.29 | | | | to | |
| L. Ab (vs | | | | | | | 4.50 | |
| L. Aa) | | | | | | | | |
| Wang data | | | | | | | | |
| set | | | | | | | | |
| ESR1 | 79 | 0.75 | 0.35 to | 0.47 | | | | |
| mRNA rich | | | 1.61 | | | | | |
| (vs poor) | | | | | | | | |
| PGR mRNA | 79 | 0.46 | 0.21 to | 0.055 | | | | |
| rich (vs | | | 1.02 | | | | | |
| poor) | | | | | | | | |
| KG | 79 | 4.84 | 2.13 to | 170E−06 | | | | |
| subgroups | | | 11.00 | | | | | |
| L. Ab (vs | | | | | | | | |
| L. Aa) | | | | | | | | |

*Number of patients studied
**Multivariate analysis not done for lack of annotations.

### Kinase Score and Molecular Subtypes

We then studied the association of the KS with the intrinsic molecular subtypes. We merged all data sets, including our 227 tumors, the 295 van de Vijver et al's tumors, the 414 Loi et al's tumors, and the 286 Wang et al's tumors, resulting in a total of 1222 tumors. The KS and molecular subtypes were determined for all tumors: 367 tumors were luminal A, 99 luminal B, 172 ERBB2-overexpressing, 214 basal, 161 normal-like and 209 unassigned. We computed and compared the distribution of the KS in each subtype. As shown in FIG. 3A, most of the luminal A and normal-like tumors had negative KS, while most of the basal and luminal B tumors had positive KS. All pairwise comparisons of KS between the five subtypes were significant (p<0.05; t-test; data not shown). ERBB2-overexpressing and unassigned samples were equally distributed with respect to their KS. The luminal Ab tumors displayed a median KS, intermediate between that of luminal B tumors, to which the score was closer, and that of luminal Aa tumors.

The five molecular subtypes displayed different KS. However, because the range of KS was rather large in each subtype, we studied whether the KS had any prognostic value in other subtypes than luminal A by comparing survival (log-rank test) between KS-negative and KS-positive tumors (FIG. 3A). As expected, difference was strong in luminal A cases (p=1.1E-07). No difference was seen for ERBB2-overexpressing tumors (p=0.86). There was a non significant trend (p=0.18) in luminal B tumors towards better RFS in KS-negative vs KS-positive samples. An opposite trend was observed in basal (p=0.23) with better RFS in KS-positive samples. The difference was strongly significant in normal-like tumors with 5-year RFS of 89% in KS-negative tumors and 50% in KS-positive tumors (p=3.1E-05). Interestingly, the KS could also be applied to the 209 samples not assigned to a molecular subtype by the intrinsic gene set. It classified them in two prognostic subgroups, with difference for 5-year RFS between tumors with low KS (82%) and tumors with high KS (60%, p=0.001).

### A Continuum in Luminal Breast Cancers

The luminal Ab tumors displayed an intermediate KS pattern between luminal Aa tumors and luminal B tumors (FIG. 3B). Comparison of histoclinical features between luminal Aa, luminal Ab and luminal B samples in the three public data sets confirmed this finding (Supplementary Table 6), with a significant increase from luminal Aa to luminal Ab to luminal B for pathological tumor size and rate of relapse, and a significant decrease for grade, mRNA expression level of ESR1 and PGR, and 5-year RFS. These results confirm that luminal Aa and Ab represent new clinically relevant subgroups of BCs until now unrecognized, and suggest a continuum between these three subgroups.

### REFERENCE LIST

[1] Charafe-Jauffret E, Ginestier C, Monville F, et al. How to best classify breast cancer: conventional and novel classifications (review). Int J Oncol 2005; 27:1307-13.
[2] Perou C M, Sorlie T, Eisen M B, et al. Molecular portraits of human breast tumours. Nature 2000; 406:747-52.
[3] Sorlie T, Perou C M, Tibshirani R, et al. Gene expression patterns of breast carcinomas distinguish tumor subclasses with clinical implications. Proc Natl Acad Sci USA 2001; 98:10869-74.
[4] Sorlie T, Tibshirani R, Parker J, et al. Repeated observation of breast tumor subtypes in independent gene expression data sets. Proc Natl Acad Sci USA 2003; 100:8418-23.
[5] Bertucci F, Finetti P, Rougemont J, et al. Gene expression profiling identifies molecular subtypes of inflammatory breast cancer. Cancer Res 2005; 65:2170-8.
[6] Geyer C E, Forster J, Lindquist D, et al. Lapatinib plus capecitabine for HER2-positive advanced breast cancer. N Engl J Med 2006; 355:2733-43.
[7] Hudis C A. Trastuzumab—mechanism of action and use in clinical practice. N Engl J Med 2007; 357:39-51.
[8] Manning G, Whyte D B, Martinez R, Hunter T, Sudarsanam S. The protein kinase complement of the human genome. Science 2002; 298:1912-34.
[9] Futreal P A, Coin L, Marshall M, et al. A census of human cancer genes. Nat Rev Cancer 2004; 4:177-83.
[10] Krause D S, Van Etten R A. Tyrosine kinases as targets for cancer therapy. N Engl J Med 2005; 353:172-87.
[11] Stephens P, Edkins S, Davies H, et al. A screen of the complete protein kinase gene family identifies diverse patterns of somatic mutations in human breast cancer. Nat Genet 2005; 37:590-2.
[12] Irizarry R A, Hobbs B, Collin F, et al. Exploration, normalization, and summaries of high density oligonucleotide array probe level data. Biostatistics 2003; 4:249-64.
[13] Eisen M B, Spellman P T, Brown P O, Botstein D. Cluster analysis and display of genome-wide expression patterns. Proc Natl Acad Sci USA 1998; 95:14863-8.
[14] Van de Vijver M J, He Y D, van't Veer L J, et al. A gene-expression signature as a predictor of survival in breast cancer. N Engl J Med 2002; 347:1999-2009.
[15] Wang Y, Klijn J G, Zhang Y, et al. Gene-expression profiles to predict distant metastasis of lymph-node-negative primary breast cancer. Lancet 2005; 365:671-9.
[16] Loi S, Haibe-Kains B, Desmedt C, et al. Definition of clinically distinct molecular subtypes in estrogen receptor-positive breast carcinomas through genomic grade. J Clin Oncol 2007; 25:1239-46.
[17] Bertucci F, Finetti P, Cervera N, et al. Gene expression profiling shows medullary breast cancer is a subgroup of basal breast cancers. Cancer Res 2006; 66:4636-44.
[18] Sotiriou C, Wirapati P, Loi S, et al. Gene expression profiling in breast cancer: understanding the molecular basis of histologic grade to improve prognosis. J Natl Cancer Inst 2006; 98:262-72.
[19] Ivshina A V, George J, Senko O, et al. Genetic reclassification of histologic grade delineates new clinical subtypes of breast cancer. Cancer Res 2006; 66:10292-301.
[20] Dai H, van't Veer L, Lamb J, et al. A cell proliferation signature is a marker of extremely poor outcome in a subpopulation of breast cancer patients. Cancer Res 2005; 65:4059-66.
[21] Paik S, Shak S, Tang G, et al. A multigene assay to predict recurrence of tamoxifen-treated, node-negative breast cancer. N Engl J Med 2004; 351:2817-26.
[22] Desmedt C, Sotiriou C. Proliferation: the most prominent predictor of clinical outcome in breast cancer. Cell Cycle 2006; 5:2198-202.
[23] Miglarese M R, Carlson RO. Development of new cancer therapeutic agents targeting mitosis. Expert Opin Investig Drugs 2006; 15:1411-25.
[24] Carvajal R D, Tse A, Schwartz G K. Aurora kinases: new targets for cancer therapy. Clin Cancer Res 2006; 12:6869-75.
[25] Strebhardt K, Ullrich A. Targeting polo-like kinase 1 for cancer therapy. Nat Rev Cancer 2006; 6:321-30.
[26] de Carcer G, de Castro I P, Malumbres M. Targeting cell cycle kinases for cancer therapy. Curr Med Chem 2007; 14:969-85.
[27] Finetti P., Cervera N, Charafe-Jauffret E., Chabannon C., Charpin C, Chaffanet M., Jacquemier J., Viens P., Birnbaum D., Bertucci F. Sixteen kinase gene expression identifies luminal breast cancers with poor prognosis. Cancer Res. 2008; 68: (3); 1-10.
[28] Brian D. Ripley. The R project in statistical computing. MSOR Connections. The newsletter of the LTSN Maths, Stats & OR Network., 1(1):23-25, February 2001.
[29] de Carcer et al. Targeting cell cycle kinases for cancer therapy, Current Medicinal Chemistry, 2007, Vol. 14, No. 1; 1-17.
[30] Malumbres et al. Current Opinion in genetics & Development 2007, 17:60-65.
[31] Malumbres et al. Therapeutic opportunities to control tumor cell cycles, Clin. Transl. Oncol. 2006; 8(6):1-000.
[32] Awada et al., The Pipeline of new anticancer agents for breast cancer treatment in 2003, Critical Reviews in Oncology/Hematology 48 (2003), 45-63.
[33] Carvajal D., Tse Archie, Schwartz G. Aueora kinases: new targets for cancer therapy. Clin. Cancer Res 2006; 12(23).
[34] Strebhardt K., Ullrich A. Targeting polo-like kinase 1 for cancer therapy. Nature 2006, Vol. 6, 321-330.
[35] Whitcombe, D., Theaker J., Guy, S. P., Brown, T., Little, S. (1999)—Detection of PCR products using self-probing amplicons and flourescence. Nature Biotech 17, 804-807
[36] Abravaya K, Huff J, Marshall R, Merchant B, Mullen C, Schneider G, and Robinson J (2003) Molecular beacons as diagnostic tools: technology and applications. Clin Chem Lab Med 41, 468-474

1. A method for analyzing cancer, preferably breast cancer, comprising detection of differential expression of at least one, or at least 2, or at least 3, or at least 4, or at least 5, or at least 6, or at least 7, or at least 8, or at least 9, or at least 10, or at least 11 or at least 12, or at least 13, or at least 14, or at least 15 of the 16 genes encoding serine/threonine kinases listed in Table 1, or of said 16 genes.

2. The method according to claim 1, wherein said differential gene expression separates basal and luminal A breast cancer.

3. The method according to claim 1, wherein said differential gene expression distinguishes subgroups of luminal A tumors of good or poor prognosis.

4. The method according to claim 3, wherein the subgroup of luminal A tumors of poor prognosis presents a high mitotic activity compared with other luminal A tumors.

5. A method according to claim 1, wherein said detection is performed on nucleic acids from a tissue sample.

6. A method according to claim 1, wherein said detection is performed on nucleic acids from a tumor cell line.

7. A method according to claim 1, wherein said detection is performed on DNA microarrays.

8. A polynucleotide library that molecularly characterizes a cancer comprising or corresponding to at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or to said 16 genes.

9. A polynucleotide library according to claim 8 immobilized on a solid support.

10. A polynucleotide library according to claim 9, wherein the support is selected from the group comprising at least one of nylon membrane, nitrocellulose membrane, glass slide, glass beads, membranes on glass support or silicon chip, plastic support.

11. A method according to claim 1, wherein said method is used for detecting, prognosis or diagnostic of breast cancer or for monitoring the treatment of a patient with a breast cancer comprising the implementation of the method on nucleic acids from a patient.

12. A method for analysing differential gene expression associated with cancer disease, preferably breast cancer, comprising:
a) reacting a polynucleotide sample from the patient with a polynucleotide library as defined in claim 8, and
b) detecting a reaction product of step (b).

13. The method according to claim 12 further comprising:
a) obtaining a reference polynucleotide sample,
b) reacting said reference sample with said polynucleotide library, for example by hybridising the polynucleotide sample with the polynucleotide library,
c) detecting a reference sample reaction product, and
d) comparing the amount of said polynucleotide sample reaction product to the amount of said reference sample reaction product.

14. A method for screening molecule for treating luminal A cases of poor prognosis comprising the analysis of the action of said molecule on at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 kinases listed in table 1 or their expression, or on said 16 kinases.

15. A kit comprising the polynucleotide library according to claim 8.

16. A method for predicting clinical outcome for a patient diagnosed with cancer, comprising determining the expression level of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes listed in Table 1, or all of the 16 genes of Table 1, or their expression products, in a cancer tissue or cell obtained from the patient, normalized against a control gene or genes, and compared to the amount found in a reference cancer tissue set, wherein overexpression of the group of genes predicts a poor clinical outcome.

17. The method of claim 16 wherein poor clinical outcome is measured in terms of relapse-free survival (RFS).

18. The method of claim 16 wherein said cancer is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

19. The method of claim 16 wherein said cancer is breast cancer.

20. The method of claim 16 wherein the overexpression level of AURKA (corresponding to SEQ ID NO: 17) AND/OR AURKB (corresponding to SEQ ID NO: 18) and/or PLK1 (corresponding to SEQ ID NO: 26) genes is determined.

21. The method of claim 16 wherein said expression level is determined using RNA obtained from a frozen or fresh tissue sample.

22. The method of claim 16 wherein said expression level is determined by reverse phase polymerase chain reaction (RT-PCR).

23. A method of predicting the likelihood of the recurrence of cancer following treatment in a cancer patient, comprising determining the expression level of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes listed in Table 1, or all of the 16 genes of Table 1, or their expression products, in a cancer tissue obtained from the patient, normalized against a control gene or genes, and compared to the amount found in a reference cancer tissue set, wherein overexpression of the group of genes indicates increased risk of recurrence following treatment.

24. The method of claim 23 wherein said cancer is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

25. The method of claim 23 wherein said cancer is breast cancer.

26. The method of claim 23 wherein said expression level is determined following surgical removal of cancer.

27. The method of claim 23 wherein said expression level is determined using RNA obtained from a fresh or frozen sample.

28. The method of claim 23 wherein said expression level is determined by reverse phase polymerase chain reaction (RT-PCR).

29. The method of claim 23 wherein said treatment uses a drug selected among the group consisting of: MK0457, PHA-739358, MLN8054, AZD1152, ON01910, BI2536, flavopiridol, USN-01.

30. A kit comprising one or more of (1) extraction buffer/reagents and protocol; (2) reverse transcription buffer/reagents and protocol; and (3) quantitative PCR buffer/reagents and protocol suitable for performing the method of claim 1.

31. The kit of claim 30 further comprising a data retrieval and analysis software.

32. The kit of claim 30 wherein component (2) includes pre-designed primers.

33. The kit of claim 30 wherein component (3) includes pre-designed PCR probes and primers.

34. Method for predicting therapeutic success of a given mode of treatment in a subject having cancer, comprising
(i) determining the pattern of expression levels of at least one, or at least 2, or at least 3, or at least 4, or at least 5, or at least 6, or at least 7, or at least 8, or at least 9, or at least 10, or at least 11, or at least 12, or at least 13, or at least 14, or at least 15 of the 16 genes encoding serine/threonine kinases listed in Table 1, or of said 16 genes,
(ii) comparing the pattern of expression levels determined in (i) with one or several reference pattern(s) of expression levels,
(iii) predicting therapeutic success for said given mode of treatment in said subject from the outcome of the comparison in step (ii).

35. The method of claim 34 wherein the cancer is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

36. The method of claim 34 wherein the cancer is breast cancer.

37. The method of claim 34, wherein said given mode of treatment (i) acts on cell proliferation, and/or (ii) acts on cell survival, and/or (iii) acts on cell motility; and/or (iv) comprises administration of a chemotherapeutic agent.

38. The method of claim 34, wherein said given mode of treatment is E7070, PHA-533533, hymenialdisine, NU2058 & NU6027, AZ703, BMS-387032, CYC202 (R-roscovitine), CDKi277, NU6140, PNU-252808, RO-3306, CVT-313, SU9516, Olomoucine, ZK-CDK (ZK304709), JNJ-7706621, PD0332991, PD0183812, Fascplysin, CA224, CINK4, caffeine, pentoxifylline, wortmannin, LY294002, UCN-01, debromohymenialdisine, Go6976, SB-218078, ICP-1, CEP-3891, TAT-5216A, CEP-6367, XL844, PD0166285, BI2536, ON01910, Scytonemin, wortmannin, HMN-214, cyclapolin-1, hesperadin, JNJ-7706621, PHA-680632, VX-680 (MK-0457), ZM447439, MLN8054, R763, AZD1152, CYC116, SNS-314, MKC-1693, AT9283, quinazoline derivatives, MP235, MP529, cincreasin, SP600125, Iressa (gefitnib, ZD1839, anti-EGFR, PDGFR, c-kit, Astra-Zeneca); ABX-EGFR (anti-EGFR, Abgenix/Amgen); Zamestra (FTI, J & J/Ortho-Biotech); Herceptin (anti-HER2/neu, Genentech); Avastin (bevancizumab, anti-VEGF antibody, Genentech); Tarceva (ertolinib, OSI-774, RTK inhibitor, Genentech-Roche); ZD66474 (anti-VEGFR, Astra-Zeneca); Erbitux (IMC-225, cetuximab, anti-EGFR, Imclone/BMS); Oncolar (anti-GRH, Novartis); PD-183805 (RTK inhibitor, Pfizer); EMD72000, (anti-EGFR/VEGF ab, MerckKgaA); CI-1033 (HER2/neu & EGF-R dual inhibitor, Pfizer); EGF10004; Herzyme (anti-HER2 ab, Medizyme Pharmaceuticals); Corixa (Microsphere delivery of HER2/neu vaccine, Medarex), ZM447439 (AstraZeneca, MK0457 (Merck), AZD1152 (AstraZeneca), PHA-680632, MLN8054 (Millenium Pharmaceutical), PHA739358 (Nerviano Sciences), scytonemin, BI2536, ON01910.

39. Method of claim 34, wherein a predictive algorithm is used.

40. Method of treatment of a neoplastic disease in a subject, comprising
a) predicting therapeutic success for a given mode of treatment in a subject having cancer, e.g., breast cancer by the method of claim 34,
b) treating said neoplastic disease in said patient by said mode of treatment, if said mode of treatment is predicted to be successful.

41. Method of selecting a therapy modality for a subject afflicted with a neoplastic disease, comprising
(i) obtaining a biological sample from said subject,
(ii) predicting from said sample, by the method of claim 1, therapeutic success in a subject having cancer, e.g., breast cancer, for a plurality of individual modes of treatment,
(iii) selecting a mode of treatment which is predicted to be successful in step (ii).

42. Method of claim 34, wherein the expression level is determined
with a hybridization based method, or
with a hybridization based method utilizing arrayed probes, or
with a hybridization based method utilizing individually labeled probes, or
by real time real time PCR, or (v) by assessing the expression of polypeptides, proteins or derivatives thereof, or (vi) by assessing the amount of polypeptides, proteins or derivatives thereof.


SEQUENCE LISTING (208 sequences)

SEQ ID NO: 1 - DNA, 533 bp, Artificial Sequence
  Synthetic Probe
ccctcaatctagaacgctacacaagaaatattttgtttttactcagcaggtgtgccttaacctccctattcagaaagctccacatcaataaacatgacactctgaagtgaaagtagccacgagaattgtgctacttatactggaacataatctggaggcaaggttcgactgcagtcgaaccttgcctccagattatgaaccagtataagtagcacaattctcgtggctactttcacttcagagtgtcatgtttattgatgtggagctttctgaatagggaggttaaggcacacctgctgagtaaaacaaatatttcttgtgtagcgttcttaggaatctggtgtctgtccggccccggtaggcctgttgggtttctagtcctccttaccatcatctccatatgagagtgtgaaaataggaacacgtgctctacctccatttagggatttgcttgggatacagaagaggccatgtgtctcagagctgttaagggcttatttttttaaaacattggagtcatagcatgtgtgtaa

SEQ ID NO: 2 - DNA, 567 bp, Artificial Sequence
  Synthetic Probe
gaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtccatgcgacctccctgaggaggaagacaatgtgtggcaccctggactacctgcccccagagatgattgaggggcgcatcgacaatgagaaggtggatctgtggtgcattggagtgctttgctatgagctgctggtggggaacccatttgagagtgcatcacacaacgagacctatcgccgcatcgtcaaggtggacctaaagttccccgcttctgtgcccacgggagcccaggacctcatctccaaactgctcaggcataacccctcggaacggctgcccctggcccaggtctcagcccacccttgggtccgggccaactctcggagggtgctgcctccctctgcccttcaatctgtcgcctgatggtccctgtcattcactcgggtgcgtgtgtttgt

SEQ ID NO: 3 - DNA, 432 bp, Artificial Sequence
  Synthetic Probe
gaagatgatttatctgctggcttggcactgattgacctgggtcagagtatagatatgaaactttttccaaaaggaactatattcacagcaaagtgtgaaacatctggttttcagtgtgttgagatgctcagcaacaaaccatggaactaccagatcgattactttggggttgctgcaacagtatattgcatgctctttggcacttacatgaaagtgaaaaatgaaggaggagagtgtaagcctgaaggtctttttagaaggcttcctcatttggatatgtggaatgaattttttcatgttatgttgaatattccagattgtcatcatcttccatctttggatttgttaaggcaaaagctgaagaaagtatttcaacaacactatactaacaagattagggccctacgtaataggctaattgtactgctctta

SEQ ID NO: 4 - DNA, 504 bp, Artificial Sequence
  Synthetic Probe
ttctttgtgcggattctgaatgccaatgatgaggccacagtgtctgttcttggggagcttgcagcagaaatgaatggggtttttgacactacattccaaagtcacctgaacaaagccttatggaaggtagggaagttaactagtcctggggctttgctctttcagtgagctaggcaatcaagtctcacagattgctgcctcagagcaatggttgtattgtggaacactgaaactgtatgtgctgtaatttaatttaggacacatttagatgcactaccattgctgttctactttttggtacaggtatattttgacgtcactgatattttttatacagtgatatacttactcatggccttgtctaacttttgtgaagaactattttattctaaacagactcattacaaatggttaccttgttatttaacccatttgtctctacttttccctgtacttttcccatttgtaatttgtaaaatgttctcttatgatcaccatgtattt

SEQ ID NO: 5 - DNA, 468 bp, Artificial Sequence
  Synthetic Probe
tgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagntgcttatcttggctttcgagtctgagtttaaaactacacattttgacatagtgtttattagcagccatc

SEQ ID NO: 6 - DNA, 361 bp, Artificial Sequence
  Synthetic Probe
tattggagatttttcctctgcgtagagccatccagatctctgtatcctgttttgactaagtcttaggtgggttgggaagacagataatgaagtaggcaaagagaaaaggacccaagatagaggtttatattcagaaatggtatatatcaatgacagcatatcaaacttcctatgggaaaaagtctggtgggtggtcagctgacagatttcccatttagtagtcatagaatacagaaatagtttagggacatgtattcattttgttattttgagcattgataggtcagtatatctacctaatctgtttggtaagtataggatatataaaccattaccattgatctgtcttatgccataatct

SEQ ID NO: 7 - DNA, 303 bp, Artificial Sequence
  Synthetic Probe
gaatcctggtgaatatagtgctgctatgttgacattattcttcctagagaagattatcctgtcctgcaaactgcaaatagtagttcctgaagtgttcacttccctgtttatccaaacatcttccaatttattttgtttgttcggcatacaaataatacctatatcttaattgtaagcaaaactttggggaaaggatgaatagaattcatttgattatttcttcatgtgtgtttagtatctgaatttgaaactcatctggtggaaaccaagtttcaggggacatgagttttccagcttttatac

SEQ ID NO: 8 - DNA, 357 bp, Artificial Sequence
  Synthetic Probe
gaaagagctaaaacgtcatcctctcttcagtgatgtggactgggaaaatctgcagcatcagactatgcctttcatcccccagccagatgatgaaacagatacctcctattttgaagccaggaatactgctcagcacctgacngtatctggatttagtctgtagcacaaaaattttccttttagtctagcctngtgttatagaatgaacttgcataattatatactccttaatactagattgatctaagggggaaagatcattatttaacctagttcaatgtgcttttaatgtacgttacagctttcacagagttaaaaggctgaaaggaatatagtcagtaatttatcttaacctca

SEQ ID NO: 9 - DNA, 397 bp, Artificial Sequence
  Synthetic Probe
atgtggtgggtatcaggaggcagcggcttaagggcgatgcctgggtttacaaaagattagtggaagacatcctatctagctgcaaggtataattgatggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtcttttttaatcatgtggttttgtatattaataattgttgactttcttagattcacttccatatgtgaatgtaagctcttaactatgtctctttgtaa

SEQ ID NO: 10 - DNA, 546 bp, Artificial Sequence
  Synthetic Probe
gctgtagtgttgaatacttggccccatgagccatgcctttctgtatagtacacatgatatttcggaattggttttactgttcttcagcaactattgtacaaaatgttcacatttaatttttctttcttcttttaagaacatattataaaaagaatactttcttggttgggcttttaatcctgtgtgtgattactagtaggaacatgagatgtgacattctaaatcttgggagaaaaaataatattaggaaaaaaatatttatgcaggaagagtagcactcactgaatagttttaaatgactgagtggtatgcttacaattgtcatgtctagatttaaattttaagtctgagattttaaatgtttttgagcttagaaaacccagttagatgcaatttggtcattaataccatgacatcttgcttataaatattccattgctctgtagttcaaatctgttagctttgtgaaaattcatcactgtgatgtttgtattctttttttttttctgtttaacagaatatgagctgtctgtcatttacctactt

SEQ ID NO: 11 - DNA, 534 bp, Artificial Sequence
  Synthetic Probe
agcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgt

SEQ ID NO: 12 - DNA, 524 bp, Artificial Sequence
  Synthetic Probe
acgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtataagttatttttgtacatgttcgggtgtgggttctacagacttgtcccc

SEQ ID NO: 13 - DNA, 398 bp, Artificial Sequence
  Synthetic Probe
taacataaagtcttcagaaagcctttctatgaaagaattttaacctataatgtaaaggatgtattctgagagaacaaagcagaatgaaacttgagtcacttactaaatatagtggatataaaatagaacacctgactttgctcttagaccataacccccgaacttactatgttcatatatttgtattgaacaatcttttaaaagcaaaaatgtaaatgatgtgtagtttatttgtgcttttattgttttccctgcgtctcagacatgttgagaatcatggacaaaacctgctggaattttggaatttttgaagatgtaaataatgtgtatttatgttataagtaacatatgtaaacatgtatatttgttttatatttatttttgtaacaccagtgtct

SEQ ID NO: 14 - DNA, 562 bp, Artificial Sequence
  Synthetic Probe
tcattgggtactcctgaaatcagacatgttcctgtagaaagaattttaagttaggctttctatgcacctatcaagaatcaagagaatagattgtatcaaacaacggcagggaaatccttcagcaattctaatccactttgggttttcagctgtttttacatctaaagcaatagactagaactgaattatcttctacatagtaaaatcacaattgtggaattctggtgatattaaggtgaaataacaaaacacaaaaggccctattttaacagttgatgtgacagtaagttttaatagaacctgtaacttcattttggaaatgcttctccaccaaataagggctttttcccctatttaaggagccagatggattgaaagatgtggaaataggcagctgtagatcttgatcttccaggtaccccatgtacctttattgagcttaattataatactgtcaaattgccacgatctcactaaaggatttctatttgctgtcagttaaaaataaagccctaaatacatttttattctttctactgagggcattgtctgttttctttgt

SEQ ID NO: 15 - DNA, 489 bp, Artificial Sequence
  Synthetic Probe
agaggatatccattcctgagctcctggctcatccatatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcagataggaggtataaaatatattggactgttatactcttgaatccctgtggaaatctacatttgaagacaacatcactctgaagtgttatcagcaaaaaaaattcagtgagattatctttaaaagaaaactgtaaaaatagcaaccacttatggcactgtatatattgtagacttgttttctctgttttatgctcttgtgtaatctacttgacatcattttactct

SEQ ID NO: 16 - DNA, 529 bp, Artificial Sequence
  Synthetic Probe
aaattggacctcagtgttgtggagaatggaggtttgaaagcaaaaacaataacaaagaagcgaaagaaagaaattgaagaaagcaaggaacctggtgttgaagatacggaatggtcaaacacacagacagaggaggccatacagacccgttcaagaaccagaaagagagtccagaagtaattcagatgctgtgaaccagatttccttttctttgttttcttttgacttttttctccttttctgttagaactgttttattttcctgtgagtcttgcgaggtggaattaatgattaaatactcatgtgttcagaaaacataaactttttttataaaaatattttgtacaattcattaaaggctaatttatgaaatttgaaaatcttcaggttatactccttaagttatcccaaagccgtgtgtttgtgatgttttggagtacatatatatgaaaattattatgacacgcacttttctaatcattgtacatttctcagagtggataaaaatgtttgacaaagtcctcacttt

SEQ ID NO: 17 - DNA, 2346 bp, Homo sapiens
acaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaa

SEQ ID NO: 18 - DNA, 3509 bp, Homo sapiens
ggcgccctgaaacgttcggcgagccgactgcggctgcgcggggtattcgaatcggcggcggcttctagtttgcggttcaggtttggccgctgccggccagcgtcctctggccatggacaccccggaaaatgtccttcagatgcttgaagcccacatgcagagctacaagggcaatgaccctcttggtgaatgggaaagatacatacagtgggtagaagagaattttcctgagaataaagaatacttgataactttactagaacatttaatgaaggaatttttagataagaagaaataccacaatgacccaagattcatcagttattgtttaaaatttgctgagtacaacagtgacctccatcaattttttgagtttctgtacaaccatgggattggaaccctgtcatcccctctgtacattgcctgggcggggcatctggaagcccaaggagagctgcagcatgccagtgctgtccttcagagaggaattcaaaaccaggctgaacccagagagttcctgcaacaacaatacaggttatttcagacacgcctcactgaaacccatttgccagctcaagctagaacctcagaacctctgcataatgttcaggttttaaatcaaatgataacatcaaaatcaaatccaggaaataacatggcctgcatttctaagaatcagggttcagagctttctggagtgatatcttcagcttgtgataaagagtcaaatatggaacgaagagtgatcacgatttctaaatcagaatattctgtgcactcatctttggcatccaaagttgatgttgagcaggttgttatgtattgcaaggagaagcttattcgtggggaatcagaattttcctttgaagaattgagagcccagaaatacaatcaacggagaaagcatgagcaatgggtaaatgaagacagacattatatgaaaaggaaagaagcaaatgcttttgaagaacagctattaaaacagaaaatggatgaacttcataagaagttgcatcaggtggtggagacatcccatgaggatctgcccgcttcccaggaaaggtccgaggttaatccagcacgtatggggccaagtgtaggctcccagcaggaactgagagcgccatgtcttccagtaacctatcagcagacaccagtgaacatggaaaagaacccaagagaggcacctcctgttgttcctcctttggcaaatgctatttctgcagctttggtgtccccagccaccagccagagcattgctcctcctgttcctttgaaagcccagacagtaacagactccatgtttgcagtggccagcaaagatgctggatgtgtgaataagagtactcatgaattcaagccacagagtggagcagagatcaaagaagggtgtgaaacacataaggttgccaacacaagttcttttcacacaactccaaacacatcactgggaatggttcaggcaacgccatccaaagtgcagccatcacccaccgtgcacacaaaagaagcattaggtttcatcatgaatatgtttcaggctcctacacttcctgatatttctgatgacaaagatgaatggcaatctctagatcaaaatgaagatgcatttgaagcccagtttcaaaaaaatgtaaggtcatctggggcttggggagtcaataagatcatctcttctttgtcatctgcttttcatgtgtttgaagatggaaacaaagaaaattatggattaccacagcctaaaaataaacccacaggagccaggacctttggagaacgctctgtcagcagacttccttcaaaaccaaaggaggaagtgcctcatgctgaagagtttttggatgactcaactgtatggggtattcgctgcaacaaaaccctggcacccagtcctaagagcccaggagacttcacatctgctgcacaacttgcgtctacaccattccacaagcttccagtggagtcagtgcacattttagaagataaagaaaatgtggtagcaaaacagtgtacccaggcgactttggattcttgtgaggaaaacatggtggtgccttcaagggatggaaaattcagtccaattcaagagaaaagcccaaaacaggccttgtcgtctcacatgtattcagcatccttacttcgtctgagccagcctgctgcaggtggggtacttacctgtgaggcagagttgggcgttgaggcttgcagactcacagacactgacgctgccattgcagaagatccaccagatgctattgctgggctccaagcagaatggatgcagatgagttcacttgggactgttgatgctccaaacttcattgttgggaacccatgggatgataagctgattttcaaacttttatctgggctttctaaaccagtgagttcctatccaaatacttttgaatggcaatgtaaacttccagccatcaagcccaagactgaatttcaattgggttctaagctggtctatgtccatcaccttcttggagaaggagcctttgcccaggtgtacgaagctacccagggagatctgaatgatgctaaaaataaacagaaatttgttttaaaggtccaaaagcctgccaacccctgggaattctacattgggacccagttgatggaaagactaaagccatctatgcagcacatgtttatgaagttctattctgcccacttattccagaatggcagtgtattagtaggagagctctacagctatggaacattattaaatgccattaacctctataaaaatacccctgaaaaagtgatgcctcaaggtcttgtcatctcttttgctatgagaatgctttacatgattgagcaagtgcatgactgtgaaatcattcatggagacattaaaccagacaatttcatacttggaaacggatttttggaacaggatgatgaagatgatttatctgctggcttggcactgattgacctgggtcagagtatagatatgaaactttttccaaaaggaactatattcacagcaaagtgtgaaacatctggttttcagtgtgttgagatgctcagcaacaaaccatggaactaccagatcgattactttggggttgctgcaacagtatattgcatgctctttggcacttacatgaaagtgaaaaatgaaggaggagagtgtaagcctgaaggtctttttagaaggcttcctcatttggatatgtggaatgaattttttcatgttatgttgaatattccagattgtcatcatcttccatctttggatttgttaaggcaaaagctgaagaaagtatttcaacaacactatactaacaagattagggccctacgtaataggctaattgtactgctcttagaatgtaagcgttcacgaaaataaaatttggatatagacagtccttaaaaatcacactgtaaatatgaatctgctcactttaaacctgtttttttttcatttattgtttatgtaaatgtttgttaaaaataaatcccatggaatatttccatgtaaaaaaaaa

SEQ ID NO: 19 - DNA, 3749 bp, Homo sapiens
aggggcgtggccacgtcgaccgcgcgggaccgttaaatttgaaacttggcggctaggggtgtgggcttgaggtggccggtttgttagggagtcgtgtacgtgccttggtcgcttctgtagctccgagggcaggttgcggaagaaagcccaggcggtctgtggcccagaggaaaggcctgcagcaggacgaggacctgagccaggaatgcaggatggcggcggtgaagaaggaagggggtgctctgagtgaagccatgtccctggagggagatgaatgggaactgagtaaagaaaatgtacaacctttaaggcaagggcggatcatgtccacgcttcagggagcactggcacaagaatctgcctgtaacaatactcttcagcagcagaaacgggcatttgaatatgaaattcgattttacactggaaatgaccctctggatgtttgggataggtatatcagctggacagagcagaactatcctcaaggtgggaaggagagtaatatgtcaacgttattagaaagagctgtagaagcactacaaggagaaaaacgatattatagtgatcctcgatttctcaatctctggcttaaattagggcgtttatgcaatgagcctttggatatgtacagttacttgcacaaccaagggattggtgtttcacttgctcagttctatatctcatgggcagaagaatatgaagctagagaaaactttaggaaagcagatgcgatatttcaggaagggattcaacagaaggctgaaccactagaaagactacagtcccagcaccgacaattccaagctcgagtgtctcggcaaactctgttggcacttgagaaagaagaagaggaggaagtttttgagtcttctgtaccacaacgaagcacactagctgaactaaagagcaaagggaaaaagacagcaagagctccaatcatccgtgtaggaggtgctctcaaggctccaagccagaacagaggactccaaaatccatttcctcaacagatgcaaaataatagtagaattactgtttttgatgaaaatgctgatgaggcttctacagcagagttgtctaagcctacagtccagccatggatagcaccccccatgcccagggccaaagagaatgagctgcaagcaggcccttggaacacaggcaggtccttggaacacaggcctcgtggcaatacagcttcactgatagctgtacccgctgtgcttcccagtttcactccatatgtggaagagactgcacaacagccagttatgacaccatgtaaaattgaacctagtataaaccacatcctaagcaccagaaagcctggaaaggaagaaggagatcctctacaaagggttcagagccatcagcaagcgtctgaggagaagaaagagaagatgatgtattgtaaggagaagatttatgcaggagtaggggaattctcctttgaagaaattcgggctgaagttttccggaagaaattaaaagagcaaagggaagccgagctattgaccagtgcagagaagagagcagaaatgcagaaacagattgaagagatggagaagaagctaaaagaaatccaaactactcagcaagaaagaacaggtgatcagcaagaagagacgatgcctacaaaggagacaactaaactgcaaattgcttccgagtctcagaaaataccaggaatgactctatccagttctgtttgtcaagtaaactgttgtgccagagaaacttcacttgcggagaacatttggcaggaacaacctcattctaaaggtcccagtgtacctttctccatttttgatgagtttcttctttcagaaaagaagaataaaagtcctcctgcagatcccccacgagttttagctcaacgaagaccccttgcagttctcaaaacctcagaaagcatcacctcaaatgaagatgtgtctccagatgtttgtgatgaatttacaggaattgaacccttgagcgaggatgccattatcacaggcttcagaaatgtaacaatttgtcctaacccagaagacacttgtgactttgccagagcagctcgttttgtatccactccttttcatgagataatgtccttgaaggatctcccttctgatcctgagagactgttaccggaagaagatctagatgtaaagacctctgaggaccagcagacagcttgtggcactatctacagtcagactctcagcatcaagaagctgagcccaattattgaagacagtcgtgaagccacacactcctctggcttctctggttcttctgcctcggttgcaagcacctcctccatcaaatgtcttcaaattcctgagaaactagaacttactaatgagacttcagaaaaccctactcagtcaccatggtgttcacagtatcgcagacagctactgaagtccctaccagagttaagtgcctctgcagagttgtgtatagaagacagaccaatgcctaagttggaaattgagaaggaaattgaattaggtaatgaggattactgcattaaacgagaatacctaatatgtgaagattacaagttattctgggtggcgccaagaaactctgcagaattaacagtaataaaggtatcttctcaacctgtcccatgggacttttatatcaacctcaagttaaaggaacgtttaaatgaagattttgatcatttttgcagctgttatcaatatcaagatggctgtattgtttggcaccaatatataaactgcttcacccttcaggatcttctccaacacagtgaatatattacccatgaaataacagtgttgattatttataaccttttgacaatagtggagatgctacacaaagcagaaatagtccatggtgacttgagtccaaggtgtctgattctcagaaacagaatccacgatccctatgattgtaacaagaacaatcaagctttgaagatagtggacttttcctacagtgttgaccttagggtgcagctggatgtttttaccctcagcggctttcggactgtacagatcctggaaggacaaaagatcctggctaactgttcttctccctaccaggtagacctgtttggtatagcagatttagcacatttactattgttcaaggaacacctacaggtcttctgggatgggtccttctggaaacttagccaaaatatttctgagctaaaagatggtgaattgtggaataaattctttgtgcggattctgaatgccaatgatgaggccacagtgtctgttcttggggagcttgcagcagaaatgaatggggtttttgacactacattccaaagtcacctgaacaaagccttatggaaggtagggaagttaactagtcctggggctttgctctttcagtgagctaggcaatcaagtctcacagattgctgcctcagagcaatggttgtattgtggaacactgaaactgtatgtgctgtaatttaatttaggacacatttagatgcactaccattgctgttctactttttggtacaggtatattttgacgtcactgatattttttatacagtgatatacttactcatggccttgtctaacttttgtgaagaactattttattctaaacagactcattacaaatggttaccttgttatttaacccatttgtctctacttttccctgtacttttcccatttgtaatttgtaaaatgttctcttatgatcaccatgtattttgtaaataataaaatagtatctgttaaatttgtgcttctaaaaaaaaa

SEQ ID NO: 20 - DNA, 1253 bp, Homo sapiens
gggcggccgggagagtagcagtgccttggaccccagctctcctccccctttctctctaaggatggcccagaaggagaactcctacccctggccctacggccgacagacggctccatctggcctgagcaccctgccccagcgagtcctccggaaagagcctgtcaccccatctgcacttgtcctcatgagccgctccaatgtccagcccacagctgcccctggccagaaggtgatggagaatagcagtgggacacccgacatcttaacgcggcacttcacaattgatgactttgagattgggcgtcctctgggcaaaggcaagtttggaaacgtgtacttggctcgggagaagaaaagccatttcatcgtggcgctcaaggtcctcttcaagtcccagatagagaaggagggcgtggagcatcagctgcgcagagagatcgaaatccaggcccacctgcaccatcccaacatcctgcgtctctacaactatttttatgaccggaggaggatctacttgattctagagtatgccccccgcggggagctctacaaggagctgcagaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtgcatgcgccctccctgaggaggaagacaatgtgtggcaccctggactacctgcccccagagatgattgaggggcgcatgcacaatgagaaggtggatctgtggtgcattggagtgctttgctatgagctgctggtggggaacccaccctttgagagtgcatcacacaacgagacctatcgccgcatcgtcaaggtggacctaaagttccccgcttccgtgcccatgggagcccaggacctcatctccaaactgctcaggcataacccctcggaacggctgcccctggcccaggtctcagcccacccttgggtccgggccaactctcggagggtgctgcctccctctgcccttcaatctgtcgcctgatggtccctgtcattcactcgggtgcgtgtgtttgtatgtctgtgtatgtataggggaaagaagggatccctaactgttcccttatctgttttctacctcctcctttgtttaataaaggctgaagctttttgtactcatgaaaaaaaaaaaaaaaaaa

SEQ ID NO: 21 - DNA, 1916 bp, Homo sapiens
gagtttgaaactgctcgcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatatcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagtttaaaactacacattttgacatagtgtttattagcagccatctaaaaaggctctaatgtatatttaactaaaattactagctttgggaattaaactgtttaacaaataaaaaaaaaaaaa

SEQ ID NO: 22 - DNA, 2035 bp, Homo sapiens
acccccacctctccctcctccttccccagtcgttcgccggaaagcatttgtctcccacctcttcataacaacaattaatttcctctggggcctgaggagggcagaatttcaaccttcggtgtgcttgggagtggcgattgtgatttacacgacaaaatgccgaggtgctcggtggagtcatggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagtgaagaaaatgtgaagtactccagttctcagccagaaccccgcacaggtctttccttatgggataccagcccctcatacattgataaattggtacaagggatcagcttttcccagcccacatgtcctgatcatatgcttttgaatagtcagttacttggcaccccaggatcctcacagaacccctggcagcggttggtcaaaagaatgacacgattctttaccaaattggatgcagacaaatcttatcaatgcctgaaagagacttgtgagaagttgggctatcaatggaagaaaagttgtatgaatcaggttactatatcaacaactgataggagaaacaataaactcattttcaaagtgaatttgttagaaatggatgataaaatattggttgacttccggctttctaagggtgatggattggagttcaagagacacttcctgaagattaaagggaagctgattgatattgtgagcagccagaagatttggcttcctgccacatgatcggaccatcggctctggggaatcctggtgaatatagtgctgctatgttgacattattcttcctagagaagattatcctgtcctgcaaactgcaaatagtagttcctgaagtgttcacttccctgtttatccaaacatcttccaatttattttgtttgttcggcatacaaataatacctatatcttaattgtaagcaaaactttggggaaaggatgaatagaattcatttgattatttcttcatgtgtgtttagtatctgaatttgaaactcatctggtggaaaccaagtttcaggggacatgagttttccagcttttatacacacgtatctcatttttatcaaaacattttgtttaattcaaaaagtacatattccatgttgatttaattctaagatgaaccaataaagacataattcttgtga

SEQ ID NO: 23 - DNA, 3222 bp, Homo sapiens
aagtgttgcgcaggcgcatccgatcgactcggtaggtggggatctcttggagacggcgacccaggcatctggggagccacagaagtcgtactcccttaaaccctgctttgctccccctgtggatgtaaccccttagctggcattttgcatctcaattggcttgtgatggaggcgtctttggggattcagatggatgagccaatggctttttctccccagcgtgaccggtttcaggctgaaggctctttaaaaaaaaacgagcagaattttaaacttgcaggtgttaaaaaagatattgagaagctttatgaagctgtaccacagcttagtaatgtgtttaagattgaggacaaaattggagaaggcactttcagctctgtttatttggccacagcacagttacaagtaggacctgaagagaaaattgctctaaaacacttgattccaacaagtcatcctataagaattgcagctgaacttcagtgcctaacagtggctggggggcaagataatgtcatgggagttaaatactgctttaggaagaatgatcatgtagttattgctatgccatatctggagcatgagtcgtttttggacattctgaattctctttcctttcaagaagtacgggaatatatgcttaatctgttcaaagctttgaaacgcattcatcagtttggtattgttcaccgtgatgttaagcccagcaattttttatataataggcgcctgaaaaagtatgccttggtagactttggtttggcccaaggaacccatgatacgaaaatagagcttcttaaatttgtccagtctgaagctcagcaggaaaggtgttcacaaaacaaatcccacataatcacaggaaacaagattccactgagtggcccagtacctaaggagctggatcagcagtccaccacaaaagcttctgttaaaagaccctacacaaatgcacaaattcagattaaacaaggaaaagacggaaaggagggatctgtaggcctttctgtccagcgctctgtttttggagaaagaaatttcaatatacacagctccatttcacatgagagccctgcagtgaaactcatgaagcagtcaaagactgtggatgtactgtctagaaagttagcaacaaaaaagaaggctatttctacaaaagttatgaatagtgctgtgatgaggaaaactgccagttcttgcccagctagcctgacctgtgactgctatgcaacagataaagtttgtagtatttgcctttcaaggcgtcagcaggttgcccctagggcaggtacaccaggattcagagcaccagaggtcttgacaaagtgccccaatcaaactacagcaattgacatgtggtctgcaggtgtcatatttctttctttgcttagtggacgatatccattttataaagcaagtgatgatttaactgctttggcccaaattatgacaattaggggatccagagaaactatccaagctgctaaaacttttgggaaatcaatattatgtagcaaagaagttccagcacaagacttgagaaaactctgtgagagactcaggggtatggattctagcactcccaagttaacaagtgatatacaagggcatgcttctcatcaaccagctatttcagagaagactgaccataaagcttcttgcctcgttcaaacacctccaggacaatactcagggaattcatttaaaaagggggatagtaatagctgtgagcattgttttgatgagtataataccaatttagaaggctggaatgaggtacctgatgaagcttatgacctgcttgataaacttctagatctaaatccagcttcaagaataacagcagaagaagctttgttgcatccattttttaaagatatgagcttgtgataatggatcttcatttaatgtttactgttatgaggtagaataaaaaagaatactttgtaatagccacaagttcttgtttagagaccagagcaggattaataatttattttaacattttagtgtttggtggcacattctaaaatatagattaagaatacttaaaatgcctgggatagttcttgggactaacaacatgatcttctttgagttaaacctacctaagtagattttaggtgggttcctattaggtcagatttttagcttccctaattacctttcactgacatatacagaaaaaggagcagttttagttttaattaattaaaattaacagatgtgatgaggattaaatgaatcaaaagacttaatttgtagattcttttagagttatgagctaggtatagtttggggaaactcaacctggtgctggtgctcttaacaattttgtaaataaagaagataatttccttttctagaggtacatattaggccttttatgaacactaaaacaatgaggaaatgttggtcatggggcaaagtatcacttaaaattgaattcatccatttttaaaaaacacttcatgaaagcattctggtgtgaattgccatttttttcttactggcttctcaattttcttccttctctgcccctacctaaaacattctcctcggaaattacatggtgctgaccacaaagtttctggatgttttattaaatattgtacgtgtttacagttgggaatttaaaataatacatacactggttgataaagggaagctgcaggaccaaggtgaagattgatagtccaaatgcttttcttttttgagttgtatattttttcacaccatcttagatataattaggtagctgctgaaaggaaaagtgaatacagaattgacggtattattggagatttttcctctgcgtagagccatccagatctctgtatcctgttttgactaagtcttaggtgggttgggaagacagataatgaagtaggcaaagagaaaaggacccaagatagaggtttatattcagaaatggtatatatcaatgacagcatatcaaacttcctatgggaaaaagtctggtgggtggtcagctgacagatttcccatttagtagtcatagaatacagaaatagtttagggacatgtattcattttgttattttgagcattgataggtcagtatatctacctaatctgtttggtaagtataggatatataaaccattaccattgatctgtcttatgccataatcttaaaaaaaatttgaatgctcttgaatttgtatattcaataaagttatccttttatattttttaaaa

SEQ ID NO: 24 - DNA, 3659 bp, Homo sapiens
ttgagcaataaggtcttttgctacaatttagtgctcttttcctcacactaaatcgaaaactctccctgttggtcctgatctgtttcagtcaggcaaattacatcctgggaaaacgtcagatgacaggggaggccactcgcttcctgctcatccagtttcgacactttctgtgctttcattagcttccagacctcagccctggccctcgctttactgtacagtcagaactggtttctacgcctcgcgagggtgggaggtcgtgtatgggaggaggaccgcttcccaccagcctcgttgggaagccaggagaaatctcttcaaatcctgcgattcagagtcaagtcccagtcgtcctttttctggtcggcccagaactgtttgtgcctcctccctcatgaggaatgatgtcagtggggccgcggtcgccgcccacgaagagtgtaaggctgcgaagtcggggctttcccgacgccccctccgtccgcgtctgcgtaggggaggtgacgagggcggggcgcggcggcggggtgacgtcacggccgcgcgcggcgtgggcggagcctcactttgaacccagttggcgggagtggctgctcgcggaggggcagtgtctgcggggccgctgtatgctgtccagcgatggatcccaccgcgggaagcaagaaggagcctggaggaggcgcggcgactgaggagggcgtgaataggatcgcagtgccaaaaccgccctccattgaggaattcagcatagtgaagcccattagccggggcgccttcgggaaagtgtatctggggcagaaaggcggcaaattgtatgcagtaaaggttgttaaaaaagcagacatgatcaacaaaaatatgactcatcaggtccaagctgagagagatgcactggcactaagcaaaagcccattcattgtccatttgtattattcactgcagtctgcaaacaatgtctacttggtaatggaatatcttattgggggagatgtcaagtctctcctacatatatatggttattttgatgaagagatggctgtgaaatatatttctgaagtagcactggctctagactaccttcacagacatggaatcatccacagggacttgaaaccggacaatatgcttatttctaatgagggtcatattaaactgacggattttggcctttcaaaagttactttgaatagagatattaatatgatggatatccttacaacaccatcaatggcaaaacctagacaagattattcaagaaccccaggacaagtgttatcgcttatcagctcgttgggatttaacacaccaattgcagaaaaaaatcaagaccctgcaaacatcctttcagcctgtctgtctgaaacatcacagctttctcaaggactcgtatgccctatgtctgtagatcaaaaggacactacgccttattctagcaaattactaaaatcatgtcttgaaacagttgcctccaacccaggaatgcctgtgaagtgtctaacttctaatttactccagtctaggaaaaggctggccacatccagtgccagtagtcaatcccacaccttcatatccagtgtggaatcagaatgccacagcagtcccaaatgggaaaaagattgccaggaaagtgatgaagcattgggcccaacaatgatgagttggaatgcagttgaaaagttatgcgcaaaatctgcaaatgccattgagacgaaaggtttcaataaaaaggatctggagttagctctttctcccattcataacagcagtgcccttcccaccactggacgctcttgtgtaaaccttgctaaaaaatgcttctctggggaagtttcttgggaagcagtagaactggatgtaaataatataaatatggacactgacacaagtcagttaggtttccatcagtcaaatcagtgggctgtggattctggtgggatatctgaagagcaccttgggaaaagaagtttaaaaagaaattttgagttggttgactccagtccttgtaaaaaaattatacagaataaaaaaacttgtgtagagtataagcataacgaaatgacaaattgttatacaaatcaaaatacaggcttaacagttgaagtgcaggaccttaagctatcagtgcacaaaagtcaacaaaatgactgtgctaataaggagaacattgtcaattcttttactgataaacaacaaacaccagaaaaattacctataccaatgatagcaaaaaaccttatgtgtgaactcgatgaagactgtgaaaagaatagtaagagggactacttaagttctagttttctatgttctgatgatgatagagcttctaaaaatatttctatgaactctgattcatcttttcctggaatttctataatggaaagtccattagaaagtcagcccttagattcagatagaagcatcaaagaatcctcttttgaagaatcaaatattgaagatccacttattgtaacaccagattgccaagaaaagacctcaccaaaaggtgtcgagaaccctgctgtacaagagagtaaccaaaaaatgttaggtcctcctttggaggtgctgaaaacgttagcctctaaaagaaatgctgttgcttttcgaagttttaacagtcatattaatgcatccaataactcagaaccatccagaatgaacatgacttctttagatgcaatggatatttcgtgtgcctacagtggttcatatcccatggctataacccctactcaaaaaagaagatcctgtatgccacatcagaccccaaatcagatcaagtcgggaactccataccgaactccgaagagtgtgagaagaggggtggcccccgttgatgatgggcgaattctaggaaccccagactaccttgcacctgagctgttactaggcagggcccatggtcctgcggtagactggtgggcacttggagtttgcttgtttgaatttctaacaggaattccccctttcaatgatgaaacaccacaacaagtattccagaatattctgaaaagagatatcccttggccagaaggtgaagaaaagttatctgataatgctcaaagtgcagtagaaatacttttaaccattgatgatacaaagagagctggaatgaaagagctaaaacgtcatcctctcttcagtgatgtggactgggaaaatctgcagcatcagactatgcctttcatcccccagccagatgatgaaacagatacctcctattttgaagccaggaatactgctcagcacctgactgtatctggatttagtctgtagcacaaaaattttccttttagtctagccttgtgttatagaatgaacttgcataattatatactccttaatactagattgatctaagggggaaagatcattatttaacctagttcaatgtgcttttaatgtacgttacagctttcacagagttaaaaggctgaaaggaatatagtcagtaatttatcttaacctcaaaactgtatataaatcttcaaagcttttttcatttatttattttgtttattgcactttatgaaaactgaagcatcaataaaattagaggacactattgagagtgagccactagcttgattttctttctcctctgatttcagttcactgttcagtttagcattaaaataataaaataatcatacagttcc

SEQ ID NO: 25 - DNA, 2130 bp, Homo sapiens
ggttaaacggggcccaaggcaggggtggcgggtcagtgctgctcgggggcttctccatccaggtccctggagttcctggtccctggagctccgcacttggcggcgcaacctgcgtgaggcagcgcgactctggcgactggccggccatgccttcccgggctgaggactatgaagtgttgtacaccattggcacaggctcctacggccgctgccagaagatccggaggaagagtgatggcaagatattagtttggaaagaacttgactatggctccatgacagaagctgagaaacagatgcttgtttctgaagtgaatttgcttcgtgaactgaaacatccaaacatcgttcgttactatgatcggattattgaccggaccaatacaacactgtacattgtaatggaatattgtgaaggaggggatctggctagtgtaattacaaagggaaccaaggaaaggcaatacttagatgaagagtttgttcttcgagtgatgactcagttgactctggccctgaaggaatgccacagacgaagtgatggtggtcataccgtattgcatcgggatctgaaaccagccaatgttttcctggatggcaagcaaaacgtcaagcttggagactttgggctagctagaatattaaaccatgacacgagttttgcaaaaacatttgttggcacaccttattacatgtctcctgaacaaatgaatcgcatgtcctacaatgagaaatcagatatctggtcattgggctgcttgctgtatgagttatgtgcattaatgcctccatttacagcttttagccagaaagaactcgctgggaaaatcagagaaggcaaattcaggcgaattccataccgttactctgatgaattgaatgaaattattacgaggatgttaaacttaaaggattaccatcgaccttctgttgaagaaattcttgagaaccctttaatagcagatttggttgcagacgagcaaagaagaaatcttgagagaagagggcgacaattaggagagccagaaaaatcgcaggattccagccctgtattgagtgagctgaaactgaaggaaattcagttacaggagcgagagcgagctctcaaagcaagagaagaaagattggagcagaaagaacaggagctttgtgttcgtgagagactagcagaggacaaactggctagagcagaaaatctgttgaagaactacagcttgctaaaggaacggaagttcctgtctctggcaagtaatccagaacttcttaatcttccatcctcagtaattaagaagaaagttcatttcagtggggaaagtaaagagaacatcatgaggagtgagaattctgagagtcagctcacatctaagtccaagtgcaaggacctgaagaaaaggcttcacgctgcccagctgcgggctcaagccctgtcagatattgagaaaaattaccaactgaaaagcagacagatcctgggcatgcgctagccaggtagagagacacagagctgtgtacaggatgtaatattaccaacctttaaagactgatattcaaatgctgtagtgttgaatacttggttccatgagccatgcctttctgtatagtacacatgatatttcggaattggttttactgttcttcagcaactattgtacaaaatgttcacatttaatttttctttcttcttttaagaacatattataaaaagaatactttcttggttgggcttttaatcctgtgtgtgattactagtaggaacatgagatgtgacattctaaatcttgggagaaaaaataatgttaggaaaaaaatatttatgcaggaagagtagcactcactgaatagttttaaatgactgagtggtatgcttacaattgtcatgtctagatttaaattttaagtctgagattttaaatgtttttgagcttagaaaacccagttagatgcaatttggtcattaataccatgacatcttgcttataaatattccattgctctgtagttcaaatctgttagctttgtgaaaattcatcactgtgatgtttgtattctttttttttttctgtttaacagaatatgagctgtctgtcatttacctacttctttcccactaaataaaagaattcttcagtt

SEQ ID NO: 26 - DNA, 2204 bp, Homo sapiens
gagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtgtaagttatttttgtacatgttcgggtgtgggttctacagccttgtccccctccccctcaaccccaccatatgaattgtacagaatatttctattgaattcggaactgtcctttccttggctttatgcacattaaacagatgtgaatattcaaaaaaaaaaaaaaaaaaaaaaaaaaaaaaaaaaa

SEQ ID NO: 27 - DNA, 2501 bp, Homo sapiens
cgaaaagattcttaggaacgccgtaccagccgcgtctctcaggacagcaggcccctgtccttctgtcgggcgccgctcagccgtgccctccgcccctcaggttctttttctaattccaaataaacttgcaagaggactatgaaagattatgatgaacttctcaaatattatgaattacatgaaactattgggacaggtggctttgcaaaggtcaaacttgcctgccatatccttactggagagatggtagctataaaaatcatggataaaaacacactagggagtgatttgccccggatcaaaacggagattgaggccttgaagaacctgagacatcagcatatatgtcaactctaccatgtgctagagacagccaacaaaatattcatggttcttgagtactgccctggaggagagctgtttgactatataatttcccaggatcgcctgtcagaagaggagacccgggttgtcttccgtcagatagtatctgctgttgcttatgtgcacagccagggctatgctcacagggacctcaagccagaaaatttgctgtttgatgaatatcataaattaaagctgattgactttggtctctgtgcaaaacccaagggtaacaaggattaccatctacagacatgctgtgggagtctggcttatgcagcacctgagttaatacaaggcaaatcatatcttggatcagaggcagatgtttggagcatgggcatactgttatatgttcttatgtgtggatttctaccatttgatgatgataatgtaatggctttatacaagaagattatgagaggaaaatatgatgttcccaagtggctctctcccagtagcattctgcttcttcaacaaatgctgcaggtggacccaaagaaacggatttctatgaaaaatctattgaaccatccctggatcatgcaagattacaactatcctgttgagtggcaaagcaagaatccttttattcacctcgatgatgattgcgtaacagaactttctgtacatcacagaaacaacaggcaaacaatggaggatttaatttcactgtggcagtatgatcacctcacggctacctatcttctgcttctagccaagaaggctcggggaaaaccagttcgtttaaggctttcttctttctcctgtggacaagccagtgctaccccattcacagacatcaagtcaaataattggagtctggaagatgtgaccgcaagtgataaaaattatgtggcgggattaatagactatgattggtgtgaagatgatttatcaacaggtgctgctactccccgaacatcacagtttaccaagtactggacagaatcaaatggggtggaatctaaatcattaactccagccttatgcagaacacctgcaaataaattaaagaacaaagaaaatgtatatactcctaagtctgctgtaaagaatgaagagtactttatgtttcctgagccaaagactccagttaataagaaccagcataagagagaaatactcactacgccaaatcgttacactacaccctcaaaagctagaaaccagtgcctgaaagaaactccaattaaaataccagtaaattcaacaggaacagacaagttaatgacaggtgtcattagccctgagaggcggtgccgctcagtggaattggatctcaaccaagcacatatggaggagactccaaaaagaaagggagccaaagtgtttgggagccttgaaagggggttggataaggttatcactgtgctcaccaggagcaaaaggaagggttctgccagagacgggcccagaagactaaagcttcactataatgtgactacaactagattagtgaatccagatcaactgttgaatgaaataatgtctattcttccaaagaagcatgttgactttgtacaaaagggttatacactgaagtgtcaaacacagtcagattttgggaaagtgacaatgcaatttgaattagaagtgtgccagcttcaaaaacccgatgtggtgggtatcaggaggcagcggcttaagggcgatgcctgggtttacaaaagattagtggaagacatcctatctagctgcaaggtataattgatggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtcttttttaatcatgtggttttgtatattaataattgttgactttcttagattcacttccatatgtgaatgtaagctcttaactatgtctctttgtaatgtgtaatttctttctgaaataaaaccatttgtgaatataaaaaaaaaaaaaaaaaaaaaaaaaaaaaaaaaaaaaaaaaaaaaaaaaaaaaaaaaaaaaaa

SEQ ID NO: 28 - DNA, 1899 bp, Homo sapiens
agcgcgcgactttttgaaagccaggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaaaaaaaaaaaaaaaaaaaaaaaaaaaaaaaaaaaaaaaaaaaaaa

SEQ ID NO: 29 - DNA, 2984 bp, Homo sapiens
ggaaattcaaacgtgtttgcggaaaggagtttgggttccatcttttcatttccccagcgcagctttctgtagaaatggaatccgaggatttaagtggcagagaattgacaattgattccataatgaacaaagtgagagacattaaaaataagtttaaaaatgaagaccttactgatgaactaagcttgaataaaatttctgctgatactacagataactcgggaactgttaaccaaattatgatgatggcaaacaacccagaggactggttgagtttgttgctcaaactagagaaaaacagtgttccgctaagtgatgctcttttaaataaattgattggtcgttacagtcaagcaattgaagcgcttcccccagataaatatggccaaaatgagagttttgctagaattcaagtgagatttgctgaattaaaagctattcaagagccagatgatgcacgtgactactttcaaatggccagagcaaactgcaagaaatttgcttttgttcatatatcttttgcacaatttgaactgtcacaaggtaatgtcaaaaaaagtaaacaacttcttcaaaaagctgtagaacgtggagcagtaccactagaaatgctggaaattgccctgcggaatttaaacctccaaaaaaagcagctgctttcagaggaggaaaagaagaatttatcagcatctacggtattaactgcccaagaatcattttccggttcacttgggcatttacagaataggaacaacagttgtgattccagaggacagactactaaagccaggtttttatatggagagaacatgccaccacaagatgcagaaataggttaccggaattcattgagacaaactaacaaaactaaacagtcatgcccatttggaagagtcccagttaaccttctaaatagcccagattgtgatgtgaagacagatgattcagttgtaccttgttttatgaaaagacaaacctctagatcagaatgccgagatttggttgtgcctggatctaaaccaagtggaaatgattcctgtgaattaagaaatttaaagtctgttcaaaatagtcatttcaaggaacctctggtgtcagatgaaaagagttctgaacttattattactgattcaataaccctgaagaataaaacggaatcaagtcttctagctaaattagaagaaactaaagagtatcaagaaccagaggttccagagagtaaccagaaacagtggcaatctaagagaaagtcagagtgtattaaccagaatcctgctgcatcttcaaatcactggcagattccggagttagcccgaaaagttaatacagagcagaaacataccacttttgagcaacctgtcttttcagtttcaaaacagtcaccaccaatatcaacatctaaatggtttgacccaaaatctatttgtaagacaccaagcagcaataccttggatgattacatgagctgttttagaactccagttgtaaagaatgactttccacctgcttgtcagttgtcaacaccttatggccaacctgcctgtttccagcagcaacagcatcaaatacttgccactccacttcaaaatttacaggttttagcatcttcttcagcaaatgaatgcatttcggttaaaggaagaatttattccattttaaagcagataggaagtggaggttcaagcaaggtatttcaggtgttaaatgaaaagaaacagatatatgctataaaatatgtgaacttagaagaagcagataaccaaactcttgatagttaccggaacgaaatagcttatttgaataaactacaacaacacagtgataagatcatccgactttatgattatgaaatcacggaccagtacatctacatggtaatggagtgtggaaatattgatcttaatagttggcttaaaaagaaaaaatccattgatccatgggaacgcaagagttactggaaaaatatgttagaggcagttcacacaatccatcaacatggcattgttcacagtgatcttaaaccagctaactttctgatagttgatggaatgctaaagctaattgattttgggattgcaaaccaaatgcaaccagatacaacaagtgttgttaaagattctcaggttggcacagttaattatatgccaccagaagcaatcaaagatatgtcttcctccagagagaatgggaaatctaagtcaaagataagccccaaaagtgatgtttggtccttaggatgtattttgtactatatgacttacgggaaaacaccatttcagcagataattaatcagatttctaaattacatgccataattgatcctaatcatgaaattgaatttcccgatattccagagaaagatcttcaagatgtgttaaagtgttgtttaaaaagggacccaaaacagaggatatccattcctgagctcctggctcatccctatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcaaataccacctataaaatatattggactgttatactcttgaatccctgtggaaatctacatttgaagacaacatcactctgaagtgttatcagcaaaaaaaattcagtagattatctttaaaagaaaactgtaaaaatagcaaccacttatggtactgtatatattgtagacttgttttctctgttttatgctcttgtgtaatctacttgacatcattttactcttggaatagtgggtggatagcaagtatattctaaaaaactttgtaaataaagttttgtggctaaaatgacactaaaaaaaaaaaaaaaaaa

SEQ ID NO: 30 - DNA, 3830 bp, Homo sapiens
gtcaccaccagcctagctcggacggcaagcggcgggagattttcaaaatgggagcccagaggcaccgcccaggcctcggaaggtgtcagggagaactttccgtggtttcagcgtcgtcgcctggagcggcggtttagagagccgagcctgatgggcgccaaggccggctggctgcttggagcgctgcctcgaagggactgcgtgaaggaagctaatccggagaacccaggccagagcctggaaatatggcgacctgcatcggggagaagatcgaggattttaaagttggaaatctgcttggtaaaggatcatttgctggtgtctacagagctgagtccattcacactggtttggaagttgcaatcaaaatgatagataagaaagccatgtacaaagcaggaatggtacagagagtccaaaatgaggtgaaaatacattgccaattgaaacatccttctatcttggagctttataactattttgaagatagcaattatgtgtatctggtattagaaatgtgccataatggagaaatgaacaggtatctaaagaatagagtgaaacccttctcagaaaatgaagctcgacacttcatgcaccagatcatcacagggatgttgtatcttcattctcatggtatactacaccgggacctcacactttctaacctcctactgactcgtaatatgaacatcaagattgctgattttgggctggcaactcaactgaaaatgccacatgaaaagcactatacattatgtggaactcctaactacatttcaccagaaattgccactcgaagtgcacatggccttgaatctgatgtttggtccctgggctgtatgttttatacattacttatcgggagaccacccttcgacactgacacagtcaagaacacattaaataaagtagtattggcagattatgaaatgccatcttttttgtcaatagaggccaaggaccttattcaccagttacttcgtagaaatccagcagatcgtttaagtctgtcttcagtattggaccatccttttatgtcccgaaattcttcaacaaaaagtaaagatttaggaactgtggaagactcaattgatagtgggcatgccacaatttctactgcaattacagcttcttccagtaccagtataagtggtagtttatttgacaaaagaagacttttgattggtcagccactcccaaataaaatgactgtatttccaaagaataaaagttcaactgatttttcttcttcaggagatggaaacagtttttatactcagtggggaaatcaagaaaccagtaatagtggaaggggaagagtaattcaagatgcagaagaaaggccacattctcgataccttcgtagagcttattcctctgatagatctggcacttctaatagtcagtctcaagcaaaaacatatacaatggaacgatgtcactcagcagaaatgctttcagtgtccaaaagatcaggaggaggtgaaaatgaagagaggtactcacccacagacaacaatgccaacatttttaacttctttaaagaaaagacatccagtagttctggatcttttgaaagacctgataacaatcaagcactctccaatcatctttgtccaggaaaaactccttttccatttgcagacccgacacctcagactgaaaccgtacaacagtggtttgggaatctgcaaataaatgctcatttaagaaaaactactgaatatgacagcatcagcccaaaccgggacttccagggccatccagatttgcagaaggacacatcaaaaaatgcctggactgatacaaaagtcaaaaagaactctgatgcttctgataatgcacattctgtaaaacagcaaaataccatgaaatatatgactgcacttcacagtaaacctgagataatccaacaagaatgtgtttttggctcagatcctctttctgaacagagcaagactaggggtatggagccaccatggggttatcagaatcgtacattaagaagcattacatctccgttggttgctcacaggttaaaaccaatcagacagaaaaccaaaaaggctgtggtgagcatacttgattcagaggaggtgtgtgtggagcttgtaaaggagtatgcatctcaagaatatgtgaaagaagttcttcagatatctagtgatggaaatacgatcactatttattatccaaatggtggtagaggttttcctcttgctgatagaccaccctcacctactgacaacatcagtaggtacagctttgacaatttaccagaaaaatactggcgaaaatatcaatatgcttccaggtttgtacagcttgtaagatctaaatctcccaaaatcacttattttacaagatatgctaaatgcattttgatggagaattctcctggtgctgattttgaggtttggttttatgatggggtaaaaatacacaaaacagaagatttcattcaggtgattgaaaagacagggaagtcttacactttaaaaagtgaaagtgaagttaatagcttgaaagaggagataaaaatgtatatggaccatgctaatgagggtcatcgtatttgtttagcactggaatccataatttcagaagaggaaaggaaaactaggagtgctccctttttcccaataatcataggaagaaaacctggtagtactagttcacctaaggccttatcacctcctccttctgtggattcaaattacccaacgagagagagagcatctttcaacagaatggtcatgcatagtgctgcttctccaacacaggcaccaatccttaatccctctatggttacaaatgaaggacttggtcttacaactacagcttctggaacagacatctcttctaatagtctaaaagattgtcttcctaaatcagcacaacttttgaaatctgtttttgtgaaaaatgttggttgggctacacagttaactagtggagctgtgtgggttcagtttaatgatgggtcccagttggttgtgcaggcaggagtgtcttctatcagttatacctcaccaaatggtcaaacaactaggtatggagaaaatgaaaaattaccagactacatcaaacagaaattacagtgtctgtcttccatccttttgatgttttctaatccgactcctaattttcattgattaaaactcctttcagacatataagtttaataaataacttttttgttgactttcaagtaaagtgattttttttaatttaacataaagtcttcagaaagcctttctatgaaagaattttaacctataatgtaaaggatgtattctgagagaacaaagcagaatgaaacttgagtcacttactaaatatagtggatataaaatagaacacctgactttgctcttagaccataacccccgaacttactatgttcatatatttgtattgaacaatcttttaaaagcaaaaatgtaaatgatgtgtagtttatttgtgcttttattgttttccctgcgtctcagacatgttgagaatcatggacaaaacctgctggaattttggaatttttgaagatgtaaataatgtgtatttatgttataagtaacatatgtaaacatgtatatttgttttatatttatttttgtaacaccagtgtctgatgaaacatttttgcaaatgcattttataaaaaaataaatatagtgataagttacattatcttttgattcatttaattaaatacttatttttaaataacttaccagtaaactcactttttaaattttgttgcctgttgaggagccaattaaattttaaatattaattttgcaaatgttaaaaaaaaaaaaaaaaaa

SEQ ID NO: 31 - DNA, 1720 bp, Homo sapiens
actgcagggtgcgaaggggccggcgccgctgccgagttacgagtcggcgaaagcggcgggaagttcgtactgggcagaacgcgacgggtctgcggcttaggtgaaaatgcctcgtgtaaaagcagctcaagctggaagacagagctctgcaaagagacatcttgcagaacaatttgcagttggagagataataactgacatggcaaaaaaggaatggaaagtaggattacccattggccaaggaggctttggctgtatatatcttgctgatatgaattcttcagagtcagttggcagtgatgcaccttgtgttgtaaaagtggaacccagtgacaatggacctctttttactgaattaaagttctaccaacgagctgcaaaaccagagcaaattcagaaatggattcgtacccgtaagctgaagtacctgggtgttcctaagtattgggggtctggtctacatgacaaaaatggaaaaagttacaggtttatgataatggatcgctttgggagtgaccttcagaaaatatatgaagcaaatgccaaaaggttttctcggaaaactgtcttgcagctaagcttaagaattctggatattctggaatatattcacgagcatgagtatgtgcatggagatatcaaggcctcaaatcttcttctgaactacaagaatcctgaccaggtgtacttggtagattatggccttgcttatcggtactgcccagaaggagttcataaagaatacaaagaagaccccaaaagatgtcacgatggcactattgaattcacgagcatcgatgcacacaatggcgtggccccatcaagacgtggtgatttggaaatacttggttattgcatgatccaatggcttactggccatcttccttgggaggataatttgaaagatcctaaatatgttagagattccaaaattagatacagagaaaatattgcaagtttgatggacaaatgttttcctgagaaaaacaaaccaggtgaaattgccaaatacatggaaacagtgaaattactagactacactgaaaaacctctttatgaaaatttacgtgacattcttttgcaaggactaaaagctataggaagtaaggatgatggcaaattggacctcagtgttgtggagaatggaggtttgaaagcaaaaacaataacaaagaagcgaaagaaagaaattgaagaaagcaaggaacctggtgttgaagatacggaatggtcaaacacacagacagaggaggccatacagacccgttcaagaaccagaaagagagtccagaagtaattcagatgctgtgaaccagatttccttttctttgttttcttttgacttttttctccttttctatttgaactgttttattttcctgtgagtcttgcgaggtggaagtaatgattaaatactcatgtgttcagaaaacataaactttttttataaaaatattttgtacaattcattaaaggctaatttatgaaatttgaaaatcttcaggttatactccttaagttatcccaaagccgtgtgtttgtgatgttttggagtacatatatatgaaaattattatgacacgcacttttctaatcattgtacatttctcagagtggataaaaatgtttgacaaagtcctcacttttaaggaaatgcaaagcttaaaataaaactctcttttgtttgatgcaaacacacagtaaaaaaaaaaaaaaaaaa

SEQ ID NO: 32 - DNA, 4361 bp, Homo sapiens
tcgtggggcgggggtggggcgggactgagggcggagtgtgagcgggctcggttttgggccgcggcgggagcgggagtcgccgccactcgagtgcgcaggcgcctggcgattaccggtctcaccatggagcggaaagtgcttgcgctccaggcccgaaagaaaaggaccaaggccaagaaggacaaagcccaaaggaaatctgaaactcagcaccgaggctctgctccccactctgagagtgatctaccagagcaggaagaggagattctgggatctgatgatgatgagcaagaagatcctaatgattattgtaaaggaggttatcatcttgtgaaaattggagatctattcaatgggagataccatgtgatccgaaagttaggctggggacacttttcaacagtatggttatcatgggatattcaggggaagaaatttgtggcaatgaaagtagttaaaagtgctgaacattacactgaaacagcactagatgaaatccggttgctgaagtcagttcgcaattcagaccctaatgatccaaatagagaaatggttgttcaactactagatgactttaaaatatcaggagttaatggaacacatatctgcatggtatttgaagttttggggcatcatctgctcaagtggatcatcaaatccaattatcaggggcttccactgccttgtgtcaaaaaaattattcagcaagtgttacagggtcttgattatttacataccaagtgccgtatcatccacactgacattaaaccagagaacatcttattgtcagtgaatgagcagtacattcggaggctggctgcagaagcaacagaatggcagcgatctggagctcctccgccttccggatctgcagtcagtactgctccccagcctaaaccagctgacaaaatgtcaaagaataagaagaagaaattgaagaagaagcagaagcgccaggcagaattactagagaagcgaatgcaggaaattgaggaaatggagaaagagtcgggccctgggcaaaaaagaccaaacaagcaagaagaatcagagagtcctgttgaaagacccttgaaagagaacccacctaataaaatgacccaagaaaaacttgaagagtcaagtaccattggccaggatcaaacgcttatggaacgtgatacagagggtggtgcagcagaaattaattgcaatggagtgattgaagtcattaattatactcagaacagtaataatgaaacattgagacataaagaggatctacataatgctaatgactgtgatgtccaaaatttgaatcaggaatctagtttcctaagctcccaaaatggagacagcagcacatctcaagaaacagactcttgtacacctataacatctgaggtgtcagacaccatggtgtgccagtcttcctcaactgtaggtcagtcattcagtgaacaacacattagccaacttcaagaaagcattcgggcagagataccctgtgaagatgaacaagagcaagaacataacggaccactggacaacaaaggaaaatccacggctggaaattttcttgttaatccccttgagccaaaaaatgcagaaaagctcaaggtgaagattgctgaccttggaaatgcttgttgggtgcacaaacatttcactgaagatattcaaacaaggcaatatcgttccttggaagttctaatcggatctggctataatacccctgctgacatttggagcacggcatgcatggcctttgaactggccacaggtgactatttgtttgaacctcattcaggggaagagtacactcgagatgaagatcacattgcattgatcatagaacttctggggaaggtgcctcgcaagctcattgtggcaggaaaatattccaaggaatttttcaccaaaaaaggtgacctgaaacatatcacgaagctgaaaccttggggcctttttgaggttctagtggagaagtatgagtggtcgcaggaagaggcagctggcttcacagatttcttactgcccatgttggagctgatccctgagaagagagccactgccgccgagtgtctccggcacccttggcttaactcctaagcccctgcccagcaccacagcagagatcacacactgaccctccgcccttccccttcaagcattttcctcttcccttttcagggtgaagctcttccttcaagagtttctagatcttgttttttttttaatccaacatgttcatttgggtttgcttacttgaccctgtggagatccccacagccattgggcatcctaggtgaatttggccttggttgggctctgccaaagactaatggactaaaatgtgaaacagcctcttgccctgtacctttccttcccattaggacatcctttaaattataagcatcctttttgaaaagagctatgaaggtgtatgagcccatccttttattcattgactctaagagtcaaattttctagtgcatatcctattgccagcataaggatgaggagggggaaagggtcttaattctatgtacagcagagacattaaacttgctgtgtccgggctgcatcatcttcctggactgtttctgttgttctctgtgttcacattttttcctgcaacttttaagctactgtcttttttaaatagctatatgaacaccaaatttgggtaccattttatcactgttcaaagcactgtcaaattcctttcatcctttaatagttaagatctttgaatcttcagtctgatttttaatgtaagcaaaaacagaaccattgaatagtaatttcttgagaacctcaggtgttctataaacagtcctttcctgtatgtcttctattaccctaagaccagagttattttggttggttgttttgttttattttttgtttttgtatccatggctggcactttactcattgcacttgagtttattgccccataactaaaggatcaggatgatggtagaacggagatctgggtttcagagctttcccatttaagaaaaatagatcttgagattctaattcttttccaaacagtcccctgctttcatgtacagctttttctttaccttacccaaaattctggccttgaagcagttttcctctatggctttgcctttctgattttctcagaggctcgagtctttaatataaccccaaatgaaagaaccaaggggaggggtgggatggcacttttttttgttggtcttgttttgttttgttttttggttggttggttcgttattttttaagattagccattctctgctgctatttccctacataatgtcaatttttaaccataattttgacatgattgagatgtacttgaggcttttttgttttaattgagaaaagactttgcaattttttttttaggatgagcctctcctagacttgacctagaatattacatattcctccagtaagtaatactgaagagcaaaagagaggcaggattggggtcacagccgcttcttcagcatggaccaagtgggccttggggattgcagcgttctcgaagtggctgtaggactcgaatttacagaaagccacagaggtgcaacttgaggctctgctagcaagccaccagtgaggctattgggtaaccacctttctatacaggagattggaatctactttgtcatttatccaccacagtgacaaaggaaaagtggtgccgttatgcaatccatttaactcataaacatattactctgagtaactggccagccattcatcggatccttcattgggtactcctgaaatcagacatgttcctgtagaaagaattttaagttaggctttctatgcacctatcaagaatcaagagaatagattgtatcaaacaacggcagggaaatccttcagcaattctaatccactttgggttttcagctgtttttacatctaaagcaatagactagaactgaattatcttctacatagtaaaatcacaattgtggaattacaggaattctggtgatattaaggtgaaataacaaaacacaaaaggccctattttaacagttgatgtgacagtaagttttaatagaacctgtaacttcattttggaaatgcttctccaccaaataagggctttttcccctatttaaggagccagatggattgaaagatgtggaaataggcagctgtagatcttgatcttccaggtaccccatgtacctttattgagcttaattataatactgtcaaattgccacgatctcactaaaggatttctatttgctgtcagttaaaaataaagccctaaatacatttttattctttctactgagggcattgtctgttttctttgtaaatgccgtacaataaacaaattatttaataaccta

SEQ ID NO: 33 - DNA, 25 bp, Artificial Sequence
  Synthetic Probe
ccctcaatctagaacgctacacaag

SEQ ID NO: 34 - DNA, 25 bp, Artificial Sequence
  Synthetic Probe
aaataggaacacgtgctctacctcc

SEQ ID NO: 35 - DNA, 25 bp, Artificial Sequence
  Synthetic Probe
gtgctctacctccatttagggattt

SEQ ID NO: 36 - DNA, 25 bp, Artificial Sequence
  Synthetic Probe
ctacctccatttagggatttgcttg

SEQ ID NO: 37 - DNA, 25 bp, Artificial Sequence
  Synthetic Probe
ttagggatttgcttgggatacagaa

SEQ ID NO: 38 - DNA, 25 bp, Artificial Sequence
  Synthetic Probe
gggatacagaagaggccatgtgtct

SEQ ID NO: 39 - DNA, 25 bp, Artificial Sequence
  Synthetic Probe
gaagaggccatgtgtctcagagctg

SEQ ID NO: 40 - DNA, 25 bp, Artificial Sequence
  Synthetic Probe
gaggccatgtgtctcagagctgtta

SEQ ID NO: 41 - DNA, 25 bp, Artificial Sequence
  Synthetic Probe
gtgtctcagagctgttaagggctta

SEQ ID NO: 42 - DNA, 25 bp, Artificial Sequence
  Synthetic Probe
cagagctgttaagggcttatttttt

SEQ ID NO: 43 - DNA, 25 bp, Artificial Sequence
  Synthetic Probe
cattggagtcatagcatgtgtgtaa

SEQ ID NO: 44 - DNA, 25 bp, Artificial Sequence
  Synthetic Probe
gaagagctgcacatttgacgagcag

SEQ ID NO: 45 - DNA, 25 bp, Artificial Sequence
  Synthetic Probe
tgacgagcagcgaacagccacgatc

SEQ ID NO: 46 - DNA, 25 bp, Artificial Sequence
  Synthetic Probe
gatgctctaatgtactgccatggga

SEQ ID NO: 47 - DNA, 25 bp, Artificial Sequence
  Synthetic Probe
gccagaaaatctgctcttagggctc

SEQ ID NO: 48 - DNA, 25 bp, Artificial Sequence
  Synthetic Probe
gaagacaatgtgtggcaccctggac

SEQ ID NO: 49 - DNA, 25 bp, Artificial Sequence
  Synthetic Probe
gaggggcgcatcgacaatgagaagg

SEQ ID NO: 50 - DNA, 25 bp, Artificial Sequence
  Synthetic Probe
agctgctggtggggaacccatttga

SEQ ID NO: 51 - DNA, 25 bp, Artificial Sequence
  Synthetic Probe
gaacccatttgagagtgcatcacac

SEQ ID NO: 52 - DNA, 25 bp, Artificial Sequence
  Synthetic Probe
gcatcacacaacgagacctatcgcc

SEQ ID NO: 53 - DNA, 25 bp, Artificial Sequence
  Synthetic Probe
ctcatctccaaactgctcaggcata

SEQ ID NO: 54 - DNA, 25 bp, Artificial Sequence
  Synthetic Probe
cattcactcgggtgcgtgtgtttgt

SEQ ID NO: 55 - DNA, 25 bp, Artificial Sequence
  Synthetic Probe
gaagatgatttatctgctggcttgg

SEQ ID NO: 56 - DNA, 25 bp, Artificial Sequence
  Synthetic Probe
tgctggcttggcactgattgacctg

SEQ ID NO: 57 - DNA, 25 bp, Artificial Sequence
  Synthetic Probe
gatgctcagcaacaaaccatggaac

SEQ ID NO: 58 - DNA, 25 bp, Artificial Sequence
  Synthetic Probe
gaactaccagatcgattactttggg

SEQ ID NO: 59 - DNA, 25 bp, Artificial Sequence
  Synthetic Probe
attactttggggttgctgcaacagt

SEQ ID NO: 60 - DNA, 25 bp, Artificial Sequence
  Synthetic Probe
catgctctttggcacttacatgaaa

SEQ ID NO: 61 - DNA, 25 bp, Artificial Sequence
  Synthetic Probe
gagagtgtaagcctgaaggtctttt

SEQ ID NO: 62 - DNA, 25 bp, Artificial Sequence
  Synthetic Probe
ttagaaggcttcctcatttggatat

SEQ ID NO: 63 - DNA, 25 bp, Artificial Sequence
  Synthetic Probe
aatattccagattgtcatcatcttc

SEQ ID NO: 64 - DNA, 25 bp, Artificial Sequence
  Synthetic Probe
gattagggccctacgtaataggcta

SEQ ID NO: 65 - DNA, 25 bp, Artificial Sequence
  Synthetic Probe
taataggctaattgtactgctctta

SEQ ID NO: 66 - DNA, 25 bp, Artificial Sequence
  Synthetic Probe
ttctttgtgcggattctgaatgcca

SEQ ID NO: 67 - DNA, 25 bp, Artificial Sequence
  Synthetic Probe
tggggtttttgacactacattccaa

SEQ ID NO: 68 - DNA, 25 bp, Artificial Sequence
  Synthetic Probe
gttaactagtcctggggctttgctc

SEQ ID NO: 69 - DNA, 25 bp, Artificial Sequence
  Synthetic Probe
ggggctttgctctttcagtgagcta

SEQ ID NO: 70 - DNA, 25 bp, Artificial Sequence
  Synthetic Probe
gagctaggcaatcaagtctcacaga

SEQ ID NO: 71 - DNA, 25 bp, Artificial Sequence
  Synthetic Probe
gtctcacagattgctgcctcagagc

SEQ ID NO: 72 - DNA, 25 bp, Artificial Sequence
  Synthetic Probe
ggacacatttagatgcactaccatt

SEQ ID NO: 73 - DNA, 25 bp, Artificial Sequence
  Synthetic Probe
cactaccattgctgttctacttttt

SEQ ID NO: 74 - DNA, 25 bp, Artificial Sequence
  Synthetic Probe
ggtacaggtatattttgacgtcact

SEQ ID NO: 75 - DNA, 25 bp, Artificial Sequence
  Synthetic Probe
ggccttgtctaacttttgtgaagaa

SEQ ID NO: 76 - DNA, 25 bp, Artificial Sequence
  Synthetic Probe
gttctcttatgatcaccatgtattt

SEQ ID NO: 77 - DNA, 25 bp, Artificial Sequence
  Synthetic Probe
tgctaagttcaagtttcgtaatgct

SEQ ID NO: 78 - DNA, 25 bp, Artificial Sequence
  Synthetic Probe
tgaagtatttttatgctctgaatgt

SEQ ID NO: 79 - DNA, 25 bp, Artificial Sequence
  Synthetic Probe
aaatgttctcatcagtttcttgcca

SEQ ID NO: 80 - DNA, 25 bp, Artificial Sequence
  Synthetic Probe
tgttaactatacaacctggctaaag

SEQ ID NO: 81 - DNA, 25 bp, Artificial Sequence
  Synthetic Probe
gatgaatatttttctactggtattt

SEQ ID NO: 82 - DNA, 25 bp, Artificial Sequence
  Synthetic Probe
caaagatcaagggctgtccgcaaca

SEQ ID NO: 83 - DNA, 25 bp, Artificial Sequence
  Synthetic Probe
aagggctgtccgcaacagggaagaa

SEQ ID NO: 84 - DNA, 25 bp, Artificial Sequence
  Synthetic Probe
gaaagctttttgtctaagtgaattc

SEQ ID NO: 85 - DNA, 25 bp, Artificial Sequence
  Synthetic Probe
gtgaattcttatgccttggtcagag

SEQ ID NO: 86 - DNA, 25 bp, Artificial Sequence
  Synthetic Probe
cttatcttggctttcgagtctgagt

SEQ ID NO: 87 - DNA, 25 bp, Artificial Sequence
  Synthetic Probe
gacatagtgtttattagcagccatc

SEQ ID NO: 88 - DNA, 25 bp, Artificial Sequence
  Synthetic Probe
tattggagatttttcctctgcgtag

SEQ ID NO: 89 - DNA, 25 bp, Artificial Sequence
  Synthetic Probe
gatttttcctctgcgtagagccatc

SEQ ID NO: 90 - DNA, 25 bp, Artificial Sequence
  Synthetic Probe
agagccatccagatctctgtatcct

SEQ ID NO: 91 - DNA, 25 bp, Artificial Sequence
  Synthetic Probe
gatctctgtatcctgttttgactaa

SEQ ID NO: 92 - DNA, 25 bp, Artificial Sequence
  Synthetic Probe
aatgacagcatatcaaacttcctat

SEQ ID NO: 93 - DNA, 25 bp, Artificial Sequence
  Synthetic Probe
aagtctggtgggtggtcagctgaca

SEQ ID NO: 94 - DNA, 25 bp, Artificial Sequence
  Synthetic Probe
gtcagctgacagatttcccatttag

SEQ ID NO: 95 - DNA, 25 bp, Artificial Sequence
  Synthetic Probe
cagatttcccatttagtagtcatag

SEQ ID NO: 96 - DNA, 25 bp, Artificial Sequence
  Synthetic Probe
ggtcagtatatctacctaatctgtt

SEQ ID NO: 97 - DNA, 25 bp, Artificial Sequence
  Synthetic Probe
aaaccattaccattgatctgtctta

SEQ ID NO: 98 - DNA, 25 bp, Artificial Sequence
  Synthetic Probe
attgatctgtcttatgccataatct

SEQ ID NO: 99 - DNA, 25 bp, Artificial Sequence
  Synthetic Probe
gaatcctggtgaatatagtgctgct

SEQ ID NO: 100 - DNA, 25 bp, Artificial Sequence
  Synthetic Probe
gtgctgctatgttgacattattctt

SEQ ID NO: 101 - DNA, 25 bp, Artificial Sequence
  Synthetic Probe
gagaagattatcctgtcctgcaaac

SEQ ID NO: 102 - DNA, 25 bp, Artificial Sequence
  Synthetic Probe
aatagtagttcctgaagtgttcact

SEQ ID NO: 103 - DNA, 25 bp, Artificial Sequence
  Synthetic Probe
cacttccctgtttatccaaacatct

SEQ ID NO: 104 - DNA, 25 bp, Artificial Sequence
  Synthetic Probe
tatccaaacatcttccaatttattt

SEQ ID NO: 105 - DNA, 25 bp, Artificial Sequence
  Synthetic Probe
tttattttgtttgttcggcatacaa

SEQ ID NO: 106 - DNA, 25 bp, Artificial Sequence
  Synthetic Probe
tttcttcatgtgtgtttagtatctg

SEQ ID NO: 107 - DNA, 25 bp, Artificial Sequence
  Synthetic Probe
tttgaaactcatctggtggaaacca

SEQ ID NO: 108 - DNA, 25 bp, Artificial Sequence
  Synthetic Probe
aaccaagtttcaggggacatgagtt

SEQ ID NO: 109 - DNA, 25 bp, Artificial Sequence
  Synthetic Probe
gacatgagttttccagcttttatac

SEQ ID NO: 110 - DNA, 25 bp, Artificial Sequence
  Synthetic Probe
gaaagagctaaaacgtcatcctctc

SEQ ID NO: 111 - DNA, 25 bp, Artificial Sequence
  Synthetic Probe
tcctctcttcagtgatgtggactgg

SEQ ID NO: 112 - DNA, 25 bp, Artificial Sequence
  Synthetic Probe
gtggactgggaaaatctgcagcatc

SEQ ID NO: 113 - DNA, 25 bp, Artificial Sequence
  Synthetic Probe
aaaatctgcagcatcagactatgcc

SEQ ID NO: 114 - DNA, 25 bp, Artificial Sequence
  Synthetic Probe
gaaacagatacctcctattttgaag

SEQ ID NO: 115 - DNA, 25 bp, Artificial Sequence
  Synthetic Probe
gtatctggatttagtctgtagcaca

SEQ ID NO: 116 - DNA, 25 bp, Artificial Sequence
  Synthetic Probe
gaacttgcataattatatactcctt

SEQ ID NO: 117 - DNA, 25 bp, Artificial Sequence
  Synthetic Probe
tatactccttaatactagattgatc

SEQ ID NO: 118 - DNA, 25 bp, Artificial Sequence
  Synthetic Probe
aagatcattatttaacctagttcaa

SEQ ID NO: 119 - DNA, 25 bp, Artificial Sequence
  Synthetic Probe
gtacgttacagctttcacagagtta

SEQ ID NO: 120 - DNA, 25 bp, Artificial Sequence
  Synthetic Probe
tagtcagtaatttatcttaacctca

SEQ ID NO: 121 - DNA, 25 bp, Artificial Sequence
  Synthetic Probe
atgtggtgggtatcaggaggcagcg

SEQ ID NO: 122 - DNA, 25 bp, Artificial Sequence
  Synthetic Probe
ggaggcagcggcttaagggcgatgc

SEQ ID NO: 123 - DNA, 25 bp, Artificial Sequence
  Synthetic Probe
agggcgatgcctgggtttacaaaag

SEQ ID NO: 124 - DNA, 25 bp, Artificial Sequence
  Synthetic Probe
ggaagacatcctatctagctgcaag

SEQ ID NO: 125 - DNA, 25 bp, Artificial Sequence
  Synthetic Probe
gattcttccatcctgccggatgagt

SEQ ID NO: 126 - DNA, 25 bp, Artificial Sequence
  Synthetic Probe
gtgtgggtgtgatacagcctacata

SEQ ID NO: 127 - DNA, 25 bp, Artificial Sequence
  Synthetic Probe
aagactgttatgatcgctttgattt

SEQ ID NO: 128 - DNA, 25 bp, Artificial Sequence
  Synthetic Probe
gagctatcttaagaccaatatctct

SEQ ID NO: 129 - DNA, 25 bp, Artificial Sequence
  Synthetic Probe
gaatctaaatcaagcccatctgtca

SEQ ID NO: 130 - DNA, 25 bp, Artificial Sequence
  Synthetic Probe
gcccatctgtcattatgttactgtc

SEQ ID NO: 131 - DNA, 25 bp, Artificial Sequence
  Synthetic Probe
agctcttaactatgtctctttgtaa

SEQ ID NO: 132 - DNA, 25 bp, Artificial Sequence
  Synthetic Probe
gctgtagtgttgaatacttggcccc

SEQ ID NO: 133 - DNA, 25 bp, Artificial Sequence
  Synthetic Probe
tgaatacttggccccatgagccatg

SEQ ID NO: 134 - DNA, 25 bp, Artificial Sequence
  Synthetic Probe
gccatgcctttctgtatagtacaca

SEQ ID NO: 135 - DNA, 25 bp, Artificial Sequence
  Synthetic Probe
gatatttcggaattggttttactgt

SEQ ID NO: 136 - DNA, 25 bp, Artificial Sequence
  Synthetic Probe
ttggttgggcttttaatcctgtgtg

SEQ ID NO: 137 - DNA, 25 bp, Artificial Sequence
  Synthetic Probe
gtagcactcactgaatagttttaaa

SEQ ID NO: 138 - DNA, 25 bp, Artificial Sequence
  Synthetic Probe
ggtatgcttacaattgtcatgtcta

SEQ ID NO: 139 - DNA, 25 bp, Artificial Sequence
  Synthetic Probe
attaataccatgacatcttgcttat

SEQ ID NO: 140 - DNA, 25 bp, Artificial Sequence
  Synthetic Probe
aaatattccattgctctgtagttca

SEQ ID NO: 141 - DNA, 25 bp, Artificial Sequence
  Synthetic Probe
ctctgtagttcaaatctgttagctt

SEQ ID NO: 142 - DNA, 25 bp, Artificial Sequence
  Synthetic Probe
tgagctgtctgtcatttacctactt

SEQ ID NO: 143 - DNA, 25 bp, Artificial Sequence
  Synthetic Probe
agcatactatgcagcgttgggaact

SEQ ID NO: 144 - DNA, 25 bp, Artificial Sequence
  Synthetic Probe
cagcgttgggaactaggccacctat

SEQ ID NO: 145 - DNA, 25 bp, Artificial Sequence
  Synthetic Probe
tgaactcttctctgtatgcactaat

SEQ ID NO: 146 - DNA, 25 bp, Artificial Sequence
  Synthetic Probe
agaccctaaagatcgtccttctgct

SEQ ID NO: 147 - DNA, 25 bp, Artificial Sequence
  Synthetic Probe
atgtctagtgatcatctcagctgaa

SEQ ID NO: 148 - DNA, 25 bp, Artificial Sequence
  Synthetic Probe
gtgtggcttgcgtaaataactgttt

SEQ ID NO: 149 - DNA, 25 bp, Artificial Sequence
  Synthetic Probe
gaggaccatagtttcttgttaacat

SEQ ID NO: 150 - DNA, 25 bp, Artificial Sequence
  Synthetic Probe
aagcacttggaattgtactgggttt

SEQ ID NO: 151 - DNA, 25 bp, Artificial Sequence
  Synthetic Probe
gtactttgatactgctcatgctgac

SEQ ID NO: 152 - DNA, 25 bp, Artificial Sequence
  Synthetic Probe
tgctcatgctgacttaaaacactag

SEQ ID NO: 153 - DNA, 25 bp, Artificial Sequence
  Synthetic Probe
ggatctactgacattagcactttgt

SEQ ID NO: 154 - DNA, 25 bp, Artificial Sequence
  Synthetic Probe
acgccgcgcgaaggtgatgagctcg

SEQ ID NO: 155 - DNA, 25 bp, Artificial Sequence
  Synthetic Probe
acctcagcaacggcagcgtgcagat

SEQ ID NO: 156 - DNA, 25 bp, Artificial Sequence
  Synthetic Probe
atcaacttcttccaggatcacacca

SEQ ID NO: 157 - DNA, 25 bp, Artificial Sequence
  Synthetic Probe
gatcacaccaagctcatcttgtgcc

SEQ ID NO: 158 - DNA, 25 bp, Artificial Sequence
  Synthetic Probe
cactgatggcagccgtgacctacat

SEQ ID NO: 159 - DNA, 25 bp, Artificial Sequence
  Synthetic Probe
gagaagcgggacttccgcacatacc

SEQ ID NO: 160 - DNA, 25 bp, Artificial Sequence
  Synthetic Probe
accgcctgagtctcctggaggagta

SEQ ID NO: 161 - DNA, 25 bp, Artificial Sequence
  Synthetic Probe
tacgcccgcactatggtggacaagc

SEQ ID NO: 162 - DNA, 25 bp, Artificial Sequence
  Synthetic Probe
gtctcaaggcctcctaatagctgcc

SEQ ID NO: 163 - DNA, 25 bp, Artificial Sequence
  Synthetic Probe
gtggctgggcagagctgcatcatcc

SEQ ID NO: 164 - DNA, 25 bp, Artificial Sequence
  Synthetic Probe
gtgtgggttctacagacttgtcccc

SEQ ID NO: 165 - DNA, 25 bp, Artificial Sequence
  Synthetic Probe
taacataaagtcttcagaaagcctt

SEQ ID NO: 166 - DNA, 25 bp, Artificial Sequence
  Synthetic Probe
tgtaaaggatgtattctgagagaac

SEQ ID NO: 167 - DNA, 25 bp, Artificial Sequence
  Synthetic Probe
gcagaatgaaacttgagtcacttac

SEQ ID NO: 168 - DNA, 25 bp, Artificial Sequence
  Synthetic Probe
gaaacttgagtcacttactaaatat

SEQ ID NO: 169 - DNA, 25 bp, Artificial Sequence
  Synthetic Probe
aatagaacacctgactttgctctta

SEQ ID NO: 170 - DNA, 25 bp, Artificial Sequence
  Synthetic Probe
gactttgctcttagaccataacccc

SEQ ID NO: 171 - DNA, 25 bp, Artificial Sequence
  Synthetic Probe
taacccccgaacttactatgttcat

SEQ ID NO: 172 - DNA, 25 bp, Artificial Sequence
  Synthetic Probe
tgtattgaacaatcttttaaaagca

SEQ ID NO: 173 - DNA, 25 bp, Artificial Sequence
  Synthetic Probe
tttccctgcgtctcagacatgttga

SEQ ID NO: 174 - DNA, 25 bp, Artificial Sequence
  Synthetic Probe
gaatcatggacaaaacctgctggaa

SEQ ID NO: 175 - DNA, 25 bp, Artificial Sequence
  Synthetic Probe
atttatttttgtaacaccagtgtct

SEQ ID NO: 176 - DNA, 25 bp, Artificial Sequence
  Synthetic Probe
tcattgggtactcctgaaatcagac

SEQ ID NO: 177 - DNA, 25 bp, Artificial Sequence
  Synthetic Probe
gttaggctttctatgcacctatcaa

SEQ ID NO: 178 - DNA, 25 bp, Artificial Sequence
  Synthetic Probe
gggaaatccttcagcaattctaatc

SEQ ID NO: 179 - DNA, 25 bp, Artificial Sequence
  Synthetic Probe
aaggccctattttaacagttgatgt

SEQ ID NO: 180 - DNA, 25 bp, Artificial Sequence
  Synthetic Probe
aatgcttctccaccaaataagggct

SEQ ID NO: 181 - DNA, 25 bp, Artificial Sequence
  Synthetic Probe
aataggcagctgtagatcttgatct

SEQ ID NO: 182 - DNA, 25 bp, Artificial Sequence
  Synthetic Probe
tgatcttccaggtaccccatgtacc

SEQ ID NO: 183 - DNA, 25 bp, Artificial Sequence
  Synthetic Probe
aatactgtcaaattgccacgatctc

SEQ ID NO: 184 - DNA, 25 bp, Artificial Sequence
  Synthetic Probe
aaggatttctatttgctgtcagtta

SEQ ID NO: 185 - DNA, 25 bp, Artificial Sequence
  Synthetic Probe
tattctttctactgagggcattgtc

SEQ ID NO: 186 - DNA, 25 bp, Artificial Sequence
  Synthetic Probe
tgagggcattgtctgttttctttgt

SEQ ID NO: 187 - DNA, 25 bp, Artificial Sequence
  Synthetic Probe
agaggatatccattcctgagctcct

SEQ ID NO: 188 - DNA, 25 bp, Artificial Sequence
  Synthetic Probe
tgagctcctggctcatccatatgtt

SEQ ID NO: 189 - DNA, 25 bp, Artificial Sequence
  Synthetic Probe
gaaatatgttctgggccaacttgtt

SEQ ID NO: 190 - DNA, 25 bp, Artificial Sequence
  Synthetic Probe
gccaacttgttggtctgaattctcc

SEQ ID NO: 191 - DNA, 25 bp, Artificial Sequence
  Synthetic Probe
tctcctaactccattttgaaagctg

SEQ ID NO: 192 - DNA, 25 bp, Artificial Sequence
  Synthetic Probe
tgaaagtcataattcttcatcctcc

SEQ ID NO: 193 - DNA, 25 bp, Artificial Sequence
  Synthetic Probe
gactgttatactcttgaatccctgt

SEQ ID NO: 194 - DNA, 25 bp, Artificial Sequence
  Synthetic Probe
atagcaaccacttatggcactgtat

SEQ ID NO: 195 - DNA, 25 bp, Artificial Sequence
  Synthetic Probe
tattgtagacttgttttctctgttt

SEQ ID NO: 196 - DNA, 25 bp, Artificial Sequence
  Synthetic Probe
gttttatgctcttgtgtaatctact

SEQ ID NO: 197 - DNA, 25 bp, Artificial Sequence
  Synthetic Probe
aatctacttgacatcattttactct

SEQ ID NO: 198 - DNA, 25 bp, Artificial Sequence
  Synthetic Probe
aaattggacctcagtgttgtggaga

SEQ ID NO: 199 - DNA, 25 bp, Artificial Sequence
  Synthetic Probe
gaacctggtgttgaagatacggaat

SEQ ID NO: 200 - DNA, 25 bp, Artificial Sequence
  Synthetic Probe
gatacggaatggtcaaacacacaga

SEQ ID NO: 201 - DNA, 25 bp, Artificial Sequence
  Synthetic Probe
acagacagaggaggccatacagacc

SEQ ID NO: 202 - DNA, 25 bp, Artificial Sequence
  Synthetic Probe
ccatacagacccgttcaagaaccag

SEQ ID NO: 203 - DNA, 25 bp, Artificial Sequence
  Synthetic Probe
tcagatgctgtgaaccagatttcct

SEQ ID NO: 204 - DNA, 25 bp, Artificial Sequence
  Synthetic Probe
gtgagtcttgcgaggtggaattaat

SEQ ID NO: 205 - DNA, 25 bp, Artificial Sequence
  Synthetic Probe
tactccttaagttatcccaaagccg

SEQ ID NO: 206 - DNA, 25 bp, Artificial Sequence
  Synthetic Probe
atcccaaagccgtgtgtttgtgatg

SEQ ID NO: 207 - DNA, 25 bp, Artificial Sequence
  Synthetic Probe
gacacgcacttttctaatcattgta

SEQ ID NO: 208 - DNA, 25 bp, Artificial Sequence
  Synthetic Probe
aaatgtttgacaaagtcctcacttt